(19) United States
(12) Patent Application Publication
(10) Pub. No.: US 2011/0251173 A1
(43) Pub. Date: Oct. 13, 2011

(54) CHEMICAL COMPOUNDS

(75) Inventors: Alan Martin BIRCH, Cheshire (GB); Frederick Woolf GOLDBERG, Cheshire (GB); Andrew LEACH, Cheshire (GB)

(73) Assignee: AstraZeneca AB, Sodertalje (SE)

(21) Appl. No.: 13/077,967

(22) Filed: Mar. 31, 2011

Related U.S. Application Data
(60) Provisional application No. 61/319,946, filed on Apr. 1, 2010.
(60) Provisional application No. 61/368,040, filed on Jul. 27, 2010.

(51) Int. Cl. A61K 31/553; A61P 3/10; A61P 3/04; C07D 498/04

(52) U.S. Cl. 514/210.18; 540/490; 514/211.05

(57) ABSTRACT

DGAT-1 inhibitor compounds of formula (I), pharmaceutically-acceptable salts and pro-drugs thereof are described, together with pharmaceutical compositions, processes for making them and their use in treating, for example, diabetes and obesity

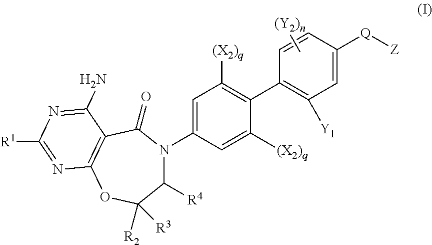

wherein, R¹, R², R³, R⁴, X₂, q, Y₁, Y₂, n, Q and Z are as defined in the description.

This application claims the benefit under 35 U.S.C. §119(e) of Application No. 61/319,946 (U.S.) filed on 1 Apr. 2010 and Application No. 61/368,040 filed on 27 Jul. 2010.

The present invention relates to compounds which inhibit acetyl CoA (acetyl coenzyme A):diacylglycerol acyltransferase (DGAT1) activity, processes for their preparation, pharmaceutical compositions containing them as the active ingredient, methods for the treatment of disease states associated with DGAT1 activity, to their use as medicaments and to their use in the manufacture of medicaments for use in the inhibition of DGAT1 in warm-blooded animals such as humans. In particular this invention relates to compounds useful for the treatment of type II diabetes, insulin resistance, impaired glucose tolerance and obesity in warm-blooded animals such as humans, more particularly to the use of these compounds in the manufacture of medicaments for use in the treatment of type II diabetes, insulin resistance, impaired glucose tolerance and obesity in warm-blooded animals such as humans.

Acyl CoA:diacylglycerol acyltransferase (DGAT) is found in the microsomal fraction of cells. It catalyzes the final reaction in the glycerol phosphate pathway, considered to be the main pathway of triglyceride synthesis in cells by facilitating the joining of a diacylglycerol with a fatty acyl CoA, resulting in the formation of triglyceride. Although it is unclear whether DGAT is rate-limiting for triglyceride synthesis, it catalyzes the only step in the pathway that is committed to producing this type of molecule [Lehner & Kuksis (1996) Biosynthesis of triacylglycerols. Prog. Lipid Res. 35: 169-201].

Two DGAT genes have been cloned and characterised. Both of the encoded proteins catalyse the same reaction although they share no sequence homology. The DGAT1 gene was identified from sequence database searches because of its similarity to acyl CoA:cholesterol acyltransferase (ACAT) genes. [Cases et al (1998) Identification of a gene encoding an acyl CoA:diacylglycerol acyltransferase, a key enzyme in triacylglycerol synthesis. Proc. Natl. Acad. Sci. USA 95: 13018-13023]. DGAT1 activity has been found in many mammalian tissues, including adipocytes.

Because of the previous lack of molecular probes, little is known about the regulation of DGAT1. DGAT1 is known to be significantly up-regulated during adipocyte differentiation.

Studies in gene knockout mice have indicated that modulators of the activity of DGAT1 would be of value in the treatment of type II diabetes and obesity. DGAT1 knockout (Dgat1^{-/-}) mice, are viable and capable of synthesizing triglycerides, as evidenced by normal fasting serum triglyceride levels and normal adipose tissue composition. Dgat1^{-/-} mice have less adipose tissue than wild-type mice at baseline and are resistant to diet-induced obesity. Metabolic rate is ˜20% higher in Dgat1^{-/-} mice than in wild-type mice on both regular and high-fat diets [Smith et al (2000) Obesity resistance and multiple mechanisms of triglyceride synthesis in mice lacking DGAT. Nature Genetics 25: 87-90]. Increased physical activity in Dgat1^{-/-} mice partially accounts for their increased energy expenditure. The Dgat1^{-/-} mice also exhibit increased insulin sensitivity and a 20% increase in glucose disposal rate. Leptin levels are 50% decreased in the Dgat1^{-/-} mice in line with the 50% decrease in fat mass.

When Dgat1^{-/-} mice are crossed with ob/ob mice, these mice exhibit the ob/ob phenotype [Chen et al (2002) Increased insulin and leptin sensitivity in mice lacking acyl CoA:diacylglycerol acyltransferase J. Clin. Invest. 109:1049-1055] indicating that the Dgat1^{-/-} phenotype requires an intact leptin pathway. When Dgat1^{-/-} mice are crossed with Agouti mice a decrease in body weight is seen with normal glucose levels and 70% reduced insulin levels compared to wild type, agouti or ob/ob/Dgat1^{-/-} mice.

Transplantation of adipose tissue from Dgat1^{-/-} mice to wild type mice confers resistance to diet-induced obesity and improved glucose metabolism in these mice [Chen et al (2003) Obesity resistance and enhanced glucose metabolism in mice transplanted with white adipose tissue lacking acyl CoA:diacylglycerol acyltransferase J. Clin. Invest. 111: 1715-1722].

Various International Applications disclose compounds which inhibit DGAT-1, for example WO 2006/064189 describes certain oxadiazole compounds which inhibit DGAT-1. However, there remains a need for further DGAT-1 inhibitors possessing desirable properties, such as, for example, pharmaco-kinetic/dynamic and/or physico-chemical and/or toxicological profiles.

Accordingly, the present invention provides a compound of formula (I),

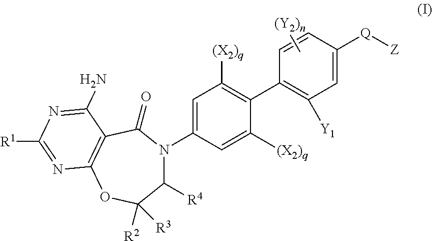

wherein
R¹ is selected from hydrogen, (1-4C)alkyl, (1-4C)alkoxy, (1-4C)perfluoroalkyl, and (1-4C)perfluoroalkoxy;
R² and R³ are independently selected from hydrogen, (1-4C)alkyl and (1-4C)perfluoroalkyl, or R² and R³ together with the carbon to which they are attached from a (3-6C)cycloalkyl ring;
R⁴ is selected from hydrogen and (1-4C)alkyl;
each q is independently 0 or 1 and each X₂ is independently selected from fluoro, chloro, bromo, amino, cyano, (1-3C)alkyl, (2-3C)alkenyl, (2-3C)alkynyl and (1-2C)alkoxy;
Y₁ is selected from hydrogen, fluoro, chloro, bromo, cyano, (1-3C)alkyl and (1-2C)alkoxy;
n is 0, 1 or 2 and each Y₂ is independently selected from fluoro, chloro, bromo, cyano, hydroxy, (1-3C)alkyl and (1-2C)alkoxy;
Q is selected from a direct bond, —(CR⁵R⁶)ₚ—, —O—(CR⁵R⁶)_{q}—, —C(O)—(CR⁵R⁶)ₜ— and —(CR⁵R⁶)ᵣ₁—O—(CR⁵R⁶)ᵣ₂— wherein p is 1, 2 or 3, q and t are independently 0, 1 or 2, r1 and r2 are independently 0 or 1, and R⁵ and R⁶ are independently selected from hydrogen, methyl and ethyl;
Z is selected from hydrogen, hydroxyl, fluoro, chloro, bromo and cyano,
   or is selected from one of the following eight groups:
(a) —CONR⁷R⁸ wherein R⁷ and R⁸ are independently selected from hydrogen, (1-3C)alkyl, —(CR⁵R⁶)ᵤ(3-5C)cycloalkyl, —(CR⁵R⁶)ₛNR⁹R¹⁰, —(CR⁵R⁶)ₛ₁—O—(CR⁵R⁶)ₛ₂NR⁹R¹⁰, —(CR⁵R⁶)ᵥ-(4- to 7-membered heterocyclyl ring) and —(CR⁵R⁶)_{w}-(5- to 7-membered heteroaryl ring) or R⁷ and R⁸ together with the nitrogen to which they are attached form a 4- to 7-membered heterocyclic ring, 7- to 8-membered spirocyclic heterocyclic ring system, or 6- to 10-membered fused bicyclic heterocyclic ring system, wherein any ring or ring system is optionally substituted with one or two groups independently selected from oxo, hydroxyl, hydroxy(1-3C)alkyl, methoxy, amino, N-(1-3C)alkylamino and N,N-di(1-3C)alkylamino;
   wherein:
   the alkyl, cycloalkyl and heterocyclyl are optionally substituted by hydroxyl, (1-4C)alkanoyl or methoxy, and the cycloalkyl and heterocyclyl are optionally substituted by (1-4C)alkyl; and the heteroaryl ring is optionally substituted by fluoro, chloro, cyano, methyl, trifluoromethyl or difluoromethyl;
   s is independently 1, 2 or 3
   s1 and s2 are independently 2 or 3;
   u, v and w are independently 0, 1, 2 or 3;
   R⁵ and R⁶ are as defined above;
   R⁹ and R¹⁰ are independently selected from hydrogen, (1-3C)alkyl, (1-6C)alkoxycarbonyl, (3-5C)cycloalkyl and a 3- to 5-membered heterocyclyl ring, or R⁹ and R¹⁰ together with the nitrogen to which they are attached form a 4- to 7-membered heterocyclic ring optionally substituted with one or two groups independently selected from (1-4C)alkyl, hydroxy(1-4C)alkyl, oxo, (1-4C)alkanoyl, hydroxy and methoxy;
(b) —SO₂NR^{7a}R^{8a}, wherein R^{7a} and R^{8a} are independently selected from hydrogen and variables defined above for R⁷ and R⁸;
(c) —S(O)ₜR⁷, wherein R⁷ is as defined above (excluding hydrogen) and t is 0, 1 or 2;
(d) —NR⁷COR⁸ wherein R⁷ and R⁸ are as defined above or R⁷ and R⁸ together form a 2-oxo-substituted 5- to 7-membered heterocyclyl ring optionally substituted with one or two substituents independently selected from hydroxyl, (1-4C)alkyl, (1-4C)alkanoyl and methoxy;
(e) —NR⁷SO₂R⁸ wherein R⁷ and R⁸ are as defined above or R⁷ and R⁸ together form a S,S-dioxo-substituted 5- to 7-membered heterocyclyl ring optionally substituted with one or two substituents independently selected from hydroxyl, (1-4C)alkyl, (1-4C)alkanoyl and methoxy;
(f) —NR⁷R⁸ wherein R⁷ and R⁸ are as defined above or R⁷ and R⁸ together with the nitrogen to which they are attached form a 5- to 7-membered heterocyclyl ring optionally substituted with one or two substituents independently selected from hydroxyl, (1-4C)alkyl, (1-4C)alkanoyl and methoxy;
(g) —OR⁷ wherein R⁷ is as defined above (excluding hydrogen);
(h) —S(O)═NR¹¹ wherein R¹¹ is H or methyl;
   and wherein any carbon atom in a linear (1-3C)alkyl, (1-3C)alkyl or (1-2C)alkoxy containing group defined above may be optionally substituted by up to 3 fluoro atoms;
   with the provisos that:
(i) within the definition of Q, when q is 0 or r2 is 0 then Z cannot be hydroxyl or —OR⁷; and
(ii) when Z is bromo or chloro then Q must be a direct bond;
   or a pharmaceutically-acceptable salt, or pro-drug thereof.

According to a further aspect of the invention there is provided a compound of formula (I) or a pharmaceutically-acceptable salt, thereof, wherein:
Z is selected from hydrogen, fluoro, chloro and bromo,
   or is selected from one of the following eight groups:
   a) —CONR⁷R⁸ wherein R⁷ and R⁸ are independently selected from —(CR⁵R⁶)ᵤ(3-5C)cycloalkyl, —(CR⁵R⁶)ₛNR⁹R¹⁰, —(CR⁵R⁶)ₛ₁—O—(CR⁵R⁶)ₛ₂NR⁹R¹⁰, —(CR⁵R⁶)ᵥ-(4- to 7-membered heterocyclyl ring) and —(CR⁵R⁶)_{w}-(5- to 7-membered heteroaryl ring) or R⁷ and R⁸ together with the nitrogen to which they are attached form a 4- to 7-membered heterocyclic ring, 7- to 8-membered spirocyclic heterocyclic ring system, or 6- to 10-membered fused bicyclic heterocyclic ring system, wherein any ring or ring system is optionally substituted with one or two groups independently selected from oxo, hydroxyl, hydroxy(1-3C)alkyl, methoxy, amino, N-(1-3C)alkylamino and N,N-di(1-3C)alkylamino;
   provided that the 5- to 7-membered heteroaryl ring is not tetrazolyl;
   wherein:
   the alkyl, cycloalkyl and heterocyclyl are optionally substituted by hydroxyl, (1-4C)alkanoyl or methoxy, and the cycloalkyl and heterocyclyl are optionally substituted by (1-4C)alkyl; and the heteroaryl ring is optionally substituted by fluoro, chloro, cyano, methyl, trifluoromethyl or difluoromethyl;
   s is independently 1, 2 or 3
   s1 and s2 are independently 2 or 3;
   u, v and w are independently 0, 1, 2 or 3;
   R⁵ and R⁶ are as defined above;
   R⁹ and R¹⁰ are independently selected from hydrogen, (1-3C)alkyl, (1-6C)alkoxycarbonyl, (3-5C)cycloalkyl and a 3- to 5-membered heterocyclyl ring, or R⁹ and R¹⁰ together with the nitrogen to which they are attached form a 4- to 7-membered heterocyclic ring optionally substituted with one or two groups independently selected from (1-4C)alkyl, hydroxy(1-4C)alkyl, oxo, (1-4C)alkanoyl, hydroxy and methoxy;
(b) —SO₂NR^{7a}R^{8a}, wherein R^{7a} and R^{8a} are independently selected from hydrogen and variables defined above for R⁷ and R⁸;
(c) —S(O)ₜR⁷, wherein R⁷ is as defined above (excluding hydrogen) and t is 0, 1 or 2;
(d) —NR⁷COR⁸ wherein R⁷ and R⁸ are as defined above or R⁷ and R⁸ together form a 2-oxo-substituted 5- to 7-membered heterocyclyl ring, optionally substituted with one or two substituents independently selected from hydroxyl, (1-4C)alkyl, (1-4C)alkanoyl and methoxy;
(e) —NR⁷SO₂R⁸ wherein R⁷ and R⁸ are as defined above or R⁷ and R⁸ together form a S,S-dioxo-substituted 5- to 7-membered heterocyclyl ring optionally substituted with one or two substituents independently selected from hydroxyl, (1-4C)alkyl, (1-4C)alkanoyl and methoxy;
(f) —NR⁷R⁸ wherein R⁷ and R⁸ are as defined above or R⁷ and R⁸ together with the nitrogen to which they are attached form a 5- to 7-membered heterocyclyl ring optionally substituted with one or two substituents independently selected from hydroxyl, (1-4C)alkyl, (1-4C)alkanoyl and methoxy;
(g) —OR⁷ wherein R⁷ is as defined above;
(h) —S(O)═NR¹¹ wherein R¹¹ is H or methyl;
   with the proviso that:
   (i) when Z is bromo or chloro then Q must be a direct bond;
      or a pharmaceutically-acceptable salt, or pro-drug thereof.

According to a further aspect of the invention there is provided a compound of formula (I) or a pharmaceutically-acceptable salt, thereof, Z is selected from hydrogen, fluoro, chloro and bromo,

or is selected from one of the following eight groups:
a) —CONR⁷R⁸ wherein R⁷ and R⁸ are independently selected from —(CR⁵R⁶)ᵤ(3-5C)cycloalkyl, —(CR⁵R⁶)ₛNR⁹R¹⁰, —(CR⁵R⁶)ₛ₁—O—(CR⁵R⁶)ₛ₂NR⁹R¹⁰ and —(CR⁵R⁶)ᵥ-(4- to 7-membered heterocyclyl ring),
   or R⁷ and R⁸ together with the nitrogen to which they are attached form a 4- to 7-membered heterocyclic ring, 7- to 8-membered spirocyclic heterocyclic ring system, or 6- to 10-membered fused bicyclic heterocyclic ring system, wherein any ring or ring system is optionally substituted with one or two groups independently selected from oxo, hydroxyl, hydroxy(1-3C)alkyl, methoxy, amino, N-(1-3C)alkylamino and N,N-di(1-3C)alkylamino;
   wherein:
   the alkyl, cycloalkyl and heterocyclyl are optionally substituted by hydroxyl, (1-4C)alkanoyl or methoxy, and the cycloalkyl and heterocyclyl are optionally substituted by (1-4C)alkyl; and the heteroaryl ring is optionally substituted by fluoro, chloro, cyano, methyl, trifluoromethyl or difluoromethyl;
   s is independently 1, 2 or 3
   s1 and s2 are independently 2 or 3;
   u, v and w are independently 0, 1, 2 or 3;
   R⁵ and R⁶ are as defined above;
   R⁹ and R¹⁰ are independently selected from hydrogen, (1-3C)alkyl, (1-6C)alkoxycarbonyl, (3-5C)cycloalkyl and a 3- to 5-membered heterocyclyl ring, or R⁹ and R¹⁰ together with the nitrogen to which they are attached form a 4- to 7-membered heterocyclic ring optionally substituted with one or two groups independently selected from (1-4C)alkyl, hydroxy(1-4C)alkyl, oxo, (1-4C)alkanoyl, hydroxy and methoxy;
(b) —SO₂NR^{7a}R^{8a}, wherein R^{7a} and R^{8a} are independently selected from hydrogen and variables defined above for R⁷ and R⁸;
(c) —S(O)ₜR⁷, wherein R⁷ is as defined above (except hydrogen) and t is 0, 1 or 2;
(d) —NR⁷COR⁸ wherein R⁷ and R⁸ are as defined above or R⁷ and R⁸ together form a 2-oxo-substituted 5- to 7-membered heterocyclyl ring, optionally substituted with one or two substituents independently selected from hydroxyl, (1-4C)alkyl, (1-4C)alkanoyl and methoxy;
(e) —NR⁷SO₂R⁸ wherein R⁷ and R⁸ are as defined above or R⁷ and R⁸ together form a S,S-dioxo-substituted 5- to 7-membered heterocyclyl ring optionally substituted with one or two substituents independently selected from hydroxyl, (1-4C)alkyl, (1-4C)alkanoyl and methoxy;
(f) —NR⁷R⁸ wherein R⁷ and R⁸ are as defined above or R⁷ and R⁸ together with the nitrogen to which they are attached form a 5- to 7-membered heterocyclyl ring optionally substituted with one or two substituents independently selected from hydroxyl, (1-4C)alkyl, (1-4C)alkanoyl and methoxy;
(g) —OR⁷ wherein R⁷ is as defined above;
(h) —S(O)═NR¹¹ wherein R¹¹ is H or methyl;
   with the proviso that:
(i) when Z is bromo or chloro then Q must be a direct bond;
   or a pharmaceutically-acceptable salt, or pro-drug thereof.

According to a further aspect of the invention there is provided a compound of formula (I) or a pharmaceutically-acceptable salt, thereof,

wherein
R¹ is selected from hydrogen, (1-4C)alkyl, (1-4C)alkoxy, (1-4C)perfluoroalkyl, and (1-4C)perfluoroalkoxy;
R² and R³ are independently selected from hydrogen, (1-4C)alkyl and (1-4C)perfluoroalkyl, or R² and R³ together with the carbon to which they are attached from a (3-6C)cycloalkyl ring;
R⁴ is selected from hydrogen and (1-4C)alkyl;
each q is independently 0 or 1 and each X₂ is independently selected from fluoro, chloro, bromo, amino, cyano, (1-3C)alkyl, (2-3C)alkenyl, (2-3C)alkynyl and (1-2C)alkoxy;
Y₁ is selected from hydrogen, fluoro, chloro, bromo, cyano, (1-3C)alkyl and (1-2C)alkoxy;
n is 0, 1 or 2 and each Y₂ is independently selected from fluoro, chloro, bromo, cyano, hydroxy, (1-3C)alkyl and (1-2C)alkoxy;
Q is selected from a direct bond, —(CR⁵R⁶)ₚ—, —O—(CR⁵R⁶)_{q}—, —C(O)—(CR⁵R⁶)ₜ— and —(CR⁵R⁶)ᵣ₁—O—(CR⁵R⁶)ᵣ₂— wherein p is 1, 2 or 3, q and t are independently 0, 1 or 2, r1 and r2 are independently 0 or 1, and R⁵ and R⁶ are independently selected from hydrogen, methyl and ethyl;
Z is selected from hydrogen, hydroxyl, fluoro, chloro, bromo, cyano, difluoromethyl and trifluoromethyl,
   or is selected from one of the following eight groups:
   (a) —CONR⁷R⁸ wherein R⁷ and R⁸ are independently selected from hydrogen, (1-3C)alkyl, —(CR⁵R⁶)ᵤ(3-5C)cycloalkyl, —(CR⁵R⁶)ₛNR⁹R¹⁰, —(CR⁵R⁶)ₛᵢ—O—(CR⁵R⁶)ₛ₂NR⁹R¹⁰, —(CR⁵R⁶)ᵥ-(4- to 7-membered heterocyclyl ring) and —(CR⁵R⁶)_{w}-(5- to 7-membered heteroaryl ring) or R⁷ and R⁸ together with the nitrogen to which they are attached form a 4- to 7-membered heterocyclic ring, 7- to 8-membered spirocyclic heterocyclic ring system, or 6- to 10-membered fused bicyclic heterocyclic ring system, wherein any ring or ring system is optionally substituted with one or two groups independently selected from oxo, hydroxyl, hydroxy(1-3C)alkyl, methoxy, amino, N-(1-3C)alkylamino and N,N-di(1-3C)alkylamino;
      wherein:
      the alkyl, cycloalkyl and heterocyclyl are optionally substituted by hydroxyl, (1-4C)alkanoyl or methoxy, and the cycloalkyl and heterocyclyl are optionally substituted by (1-4C)alkyl; and the heteroaryl ring is optionally substituted by fluoro, chloro, cyano, methyl, trifluoromethyl or difluoromethyl;
      s is independently 1, 2 or 3
      s1 and s2 are independently 2 or 3;
      u, v and w are independently 0, 1, 2 or 3;
      R⁵ and R⁶ are as defined above;
      R⁹ and R¹⁰ are independently selected from hydrogen, (1-3C)alkyl, (1-6C)alkoxycarbonyl, (3-5C)cycloalkyl and a 3- to 5-membered heterocyclyl ring, or R⁹ and R¹⁰ together with the nitrogen to which they are attached form a 4- to 7-membered heterocyclic ring optionally substituted with one or two groups independently selected from (1-4C)alkyl, hydroxy(1-4C)alkyl, oxo, (1-4C)alkanoyl, hydroxy and methoxy;
   (b) —SO₂NR^{7a}R^{8a}, wherein R^{7a} and R^{8a} are independently selected from hydrogen and variables defined above for R⁷ and R⁸;
   (c) —S(O)ₜR⁷, wherein R⁷ is as defined above (excluding hydrogen) and t is 0, 1 or 2;
   (d) —NR⁷COR⁸ wherein R⁷ and R⁸ are as defined above or R⁷ and R⁸ together form a 2-oxo-substituted 5- to 7-membered heterocyclyl ring optionally substituted with one or two substituents independently selected from hydroxyl, (1-4C)alkyl, (1-4C)alkanoyl and methoxy;
   (e) —NR⁷SO₂R⁸ wherein R⁷ and R⁸ are as defined above or R⁷ and R⁸ together form a S,S-dioxo-substituted 5- to 7-membered heterocyclyl ring optionally substituted with one or two substituents independently selected from hydroxyl, (1-4C)alkyl, (1-4C)alkanoyl and methoxy;
   (f) —NR⁷R⁸ wherein R⁷ and R⁸ are as defined above or R⁷ and R⁸ together with the nitrogen to which they are attached form a 5- to 7-membered heterocyclyl ring optionally substituted with one or two substituents independently selected from hydroxyl, (1-4C)alkyl, (1-4C)alkanoyl and methoxy;
   (g) —OR⁷ wherein R⁷ is as defined above (excluding hydrogen);
   (h) —S(O)═NR¹¹ wherein R¹¹ is H or methyl;
      and wherein any carbon atom in a linear (1-3C)alkyl, (1-3C)alkyl or (1-2C)alkoxy containing group defined above may be optionally substituted by up to 3 fluoro atoms;
      with the provisos that:
(iii) when q is 0 or r2 is 0 then Z cannot be hydroxyl or —OR⁷; and
(iv) when Z is bromo or chloro then Q must be a direct bond;
   or a pharmaceutically-acceptable salt, or pro-drug thereof.

According to a further aspect of the invention there is provided a compound of formula (I) or a pharmaceutically-acceptable salt, thereof, wherein:
Z is selected from hydrogen, hydroxyl, fluoro, chloro, bromo, cyano, difluoromethyl and trifluoromethyl,
   or is selected from one of the following eight groups:
   a) —CONR⁷R⁸ wherein R⁷ and R⁸ are independently selected from —(CR⁵R⁶)ᵤ(3-5C)cycloalkyl, —(CR⁵R⁶)ₛNR⁹R¹⁰, —(CR⁵R⁶)ₛ₁—O—(CR⁵R⁶)ₛ₂NR⁹R¹⁰, —(CR⁵R⁶)ᵥ-(4- to 7-membered heterocyclyl ring) and —(CR⁵R⁶)_{w}-(5- to 7-membered heteroaryl ring) or R⁷ and R⁸ together with the nitrogen to which they are attached form a 4- to 7-membered heterocyclic ring, 7- to 8-membered spirocyclic heterocyclic ring system, or 6- to 10-membered fused bicyclic heterocyclic ring system, wherein any ring or ring system is optionally substituted with one or two groups independently selected from oxo, hydroxyl, hydroxy(1-3C)alkyl, methoxy, amino, N-(1-3C)alkylamino and N,N-di(1-3C)alkylamino;
      provided that the 5- to 7-membered heteroaryl ring is not tetrazolyl;
      wherein:
      the alkyl, cycloalkyl and heterocyclyl are optionally substituted by hydroxyl, (1-4C)alkanoyl or methoxy, and the cycloalkyl and heterocyclyl are optionally substituted by (1-4C)alkyl; and the heteroaryl ring is optionally substituted by fluoro, chloro, cyano, methyl, trifluoromethyl or difluoromethyl;
      s is independently 1, 2 or 3
      s1 and s2 are independently 2 or 3;
      u, v and w are independently 0, 1, 2 or 3;
      R⁵ and R⁶ are as defined above;
      R⁹ and R¹⁰ are independently selected from hydrogen, (1-3C)alkyl, (1-6C)alkoxycarbonyl, (3-5C)cycloalkyl and a 3- to 5-membered heterocyclyl ring, or R⁹ and R¹⁰ together with the nitrogen to which they are attached form a 4- to 7-membered heterocyclic ring optionally substituted with one or two groups independently selected from (1-4C)alkyl, hydroxy(1-4C)alkyl, oxo, (1-4C)alkanoyl, hydroxy and methoxy;
   (b) —SO₂NR^{7a}R^{8a}, wherein R^{7a} and R^{8a} are independently selected from hydrogen and variables defined above for R⁷ and R⁸;
   (c) —S(O)ₜR⁷, wherein R⁷ is as defined above (excluding hydrogen) and t is 0, 1 or 2;
   (d) —NR⁷COR⁸ wherein R⁷ and R⁸ are as defined above or R⁷ and R⁸ together form a 2-oxo-substituted 5- to 7-membered heterocyclyl ring, optionally substituted with one or two substituents independently selected from hydroxyl, (1-4C)alkyl, (1-4C)alkanoyl and methoxy;
   (e) —NR⁷SO₂R⁸ wherein R⁷ and R⁸ are as defined above or R⁷ and R⁸ together form a S,S-dioxo-substituted 5- to 7-membered heterocyclyl ring optionally substituted with one or two substituents independently selected from hydroxyl, (1-4C)alkyl, (1-4C)alkanoyl and methoxy;
   (f) —NR⁷R⁸ wherein R⁷ and R⁸ are as defined above or R⁷ and R⁸ together with the nitrogen to which they are attached form a 5- to 7-membered heterocyclyl ring optionally substituted with one or two substituents independently selected from hydroxyl, (1-4C)alkyl, (1-4C)alkanoyl and methoxy;
   (g) —OR⁷ wherein R⁷ is as defined above;
   (h) —S(O)═NR¹¹ wherein R¹¹ is H or methyl;
      with the proviso that:
   (i) when Z is bromo or chloro then Q must be a direct bond;
      or a pharmaceutically-acceptable salt, or pro-drug thereof.

According to a further aspect of the invention there is provided a compound of formula (I) or a pharmaceutically-acceptable salt, thereof,
Z is selected from hydrogen, hydroxyl, fluoro, chloro, bromo, cyano, difluoromethyl and trifluoromethyl,
   or is selected from one of the following eight groups:
   a) —CONR⁷R⁸ wherein R⁷ and R⁸ are independently selected from —(CR⁵R⁶)ᵤ(3-5C)cycloalkyl, —(CR⁵R⁶)ₛNR⁹R¹⁰, —(CR⁵R⁶)ₛ₁—O—(CR⁵R⁶)ₛ₂NR⁹R¹⁰ and —(CR⁵R⁶)ᵥ-(4- to 7-membered heterocyclyl ring),
      or R⁷ and R⁸ together with the nitrogen to which they are attached form a 4- to 7-membered heterocyclic ring, 7- to 8-membered spirocyclic heterocyclic ring system, or 6- to 10-membered fused bicyclic heterocyclic ring system, wherein any ring or ring system is optionally substituted with one or two groups independently selected from oxo, hydroxyl, hydroxy(1-3C)alkyl, methoxy, amino, N-(1-3C)alkylamino and N,N-di(1-3C)alkylamino;
      wherein:
      the alkyl, cycloalkyl and heterocyclyl are optionally substituted by hydroxyl, (1-4C)alkanoyl or methoxy, and the cycloalkyl and heterocyclyl are optionally substituted by (1-4C)alkyl; and the heteroaryl ring is optionally substituted by fluoro, chloro, cyano, methyl, trifluoromethyl or difluoromethyl;
      s is independently 1, 2 or 3
      s1 and s2 are independently 2 or 3;
      u, v and w are independently 0, 1, 2 or 3;
      R⁵ and R⁶ are as defined above;
      R⁹ and R¹⁰ are independently selected from hydrogen, (1-3C)alkyl, (1-6C)alkoxycarbonyl, (3-5C)cycloalkyl and a 3- to 5-membered heterocyclyl ring, or R⁹ and R¹⁰ together with the nitrogen to which they are attached form a 4- to 7-membered heterocyclic ring optionally substituted with one or two groups independently selected from (1-4C)alkyl, hydroxy(1-4C)alkyl, oxo, (1-4C)alkanoyl, hydroxy and methoxy;
   (b) —SO₂NR^{7a}R^{8a}, wherein R^{7a} and R^{8a} are independently selected from hydrogen and variables defined above for R⁷ and R⁸;
   (c) —S(O)ₜR⁷, wherein R⁷ is as defined above (except hydrogen) and t is 0, 1 or 2;
   (d) —NR⁷COR⁸ wherein R⁷ and R⁸ are as defined above or R⁷ and R⁸ together form a 2-oxo-substituted 5- to 7-membered heterocyclyl ring, optionally substituted with one or two substituents independently selected from hydroxyl, (1-4C)alkyl, (1-4C)alkanoyl and methoxy;
   (e) —NR⁷SO₂R⁸ wherein R⁷ and R⁸ are as defined above or R⁷ and R⁸ together form a S,S-dioxo-substituted 5- to 7-membered heterocyclyl ring optionally substituted with one or two substituents independently selected from hydroxyl, (1-4C)alkyl, (1-4C)alkanoyl and methoxy;
   (f) —NR⁷R⁸ wherein R⁷ and R⁸ are as defined above or R⁷ and R⁸ together with the nitrogen to which they are attached form a 5- to 7-membered heterocyclyl ring optionally substituted with one or two substituents independently selected from hydroxyl, (1-4C)alkyl, (1-4C)alkanoyl and methoxy;
   (g) —OR⁷ wherein R⁷ is as defined above;
   (h) —S(O)═NR¹¹ wherein R¹¹ is H or methyl;
      with the proviso that:
   (i) when Z is bromo or chloro then Q must be a direct bond;
      or a pharmaceutically-acceptable salt, or pro-drug thereof.

According to a further aspect of the invention there is provided a compound of formula (I) or a pharmaceutically-acceptable salt, thereof, wherein
R¹ is selected from hydrogen, (1-4C)alkyl, (1-4C)alkoxy, (1-4C)perfluoroalkyl, and (1-4C)perfluoroalkoxy;
R² and R³ are independently selected from hydrogen, (1-4C)alkyl and (1-4C)perfluoroalkyl, or R² and R³ together with the carbon to which they are attached from a (3-6C)cycloalkyl ring;
R⁴ is selected from hydrogen and (1-4C)alkyl;
each q is independently 0 or 1 and each X₂ is independently selected from fluoro, chloro, bromo, amino, cyano, (1-3C)alkyl, (2-3C)alkenyl, (2-3C)alkynyl and (1-2C)alkoxy;
Y₁ is selected from hydrogen, fluoro, chloro, bromo, cyano, (1-3C)alkyl and (1-2C)alkoxy;
n is 0, 1 or 2 and each Y₂ is independently selected from fluoro, chloro, bromo, cyano, hydroxy, (1-3C)alkyl and (1-2C)alkoxy;
Q is selected from a direct bond, —(CR⁵R⁶)ₚ—, —O—(CR⁵R⁶)ᵤ—, —C(O)—(CR⁵R⁶)ₜ— and —(CR⁵R⁶)ᵣ₁—O—(CR⁵R⁶)ᵣ₂— wherein p is 1, 2 or 3, q and t are independently 0, 1 or 2, r1 and r2 are independently 0 or 1, and R⁵ and R⁶ are independently selected from hydrogen, methyl and ethyl;
Z is selected from hydrogen, hydroxyl, fluoro, chloro, bromo and cyano, or is selected from one of the following eight groups:
   (a) —CONR⁷R⁸ wherein R⁷ and R⁸ are independently selected from hydrogen, (1-3C)alkyl, —(CR⁵R⁶)ᵤ(3-5C)cycloalkyl, —(CR⁵R⁶)ₛNR⁹R¹⁰, —(CR⁵R⁶)ₛ₁—O—(CR⁵R⁶)ₛ₂NR⁹R¹⁰, —(CR⁵R⁶)ᵥ-(4- to 7-membered heterocyclyl ring) and —(CR⁵R⁶)_{w}-(5- to 7-membered heteroaryl ring) or R⁷ and R⁸ together with the nitrogen to which they are attached form a 4- to 7-membered heterocyclic ring, 7- to 8-membered spirocyclic heterocyclic ring system, or 6- to 10-membered fused bicyclic heterocyclic ring system, wherein any ring or ring system is optionally substituted with one or two groups independently selected from oxo, hydroxyl, hydroxy(1-3C)alkyl, methoxy, amino, N-(1-3C)alkylamino and N,N-di(1-3C)alkylamino;
      wherein:
      the alkyl, cycloalkyl and heterocyclyl are optionally substituted by hydroxyl, (1-4C)alkanoyl or methoxy, and the cycloalkyl and heterocyclyl are optionally substituted by (1-4C)alkyl; and the heteroaryl ring is optionally substituted by fluoro, chloro, cyano, methyl, trifluoromethyl or difluoromethyl;
      s, s1 and s2 are independently 2 or 3;
      u, v and w are independently 0, 1, 2 or 3;
      R⁵ and R⁶ are as defined above;
      R⁹ and R¹⁰ are independently selected from hydrogen, (1-3C)alkyl, (1-6C)alkoxycarbonyl, (3-5C)cycloalkyl and a 3- to 5-membered heterocyclyl ring, or R⁹ and R¹⁰ together with the nitrogen to which they are attached form a 4- to 7-membered heterocyclic ring optionally substituted with one or two groups independently selected from (1-4C)alkyl, hydroxy(1-4C)alkyl, oxo, (1-4C)alkanoyl, hydroxy and methoxy;
   (b) —SO₂NR^{7a}R^{8a}, wherein R^{7a} and R^{8a} are independently selected from hydrogen and variables defined above for R⁷ and R⁸;
   (c) —S(O)ₜR⁷, wherein R⁷ is as defined above (excluding hydrogen) and t is 0, 1 or 2;
   (d) —NR⁷COR⁸ wherein R⁷ and R⁸ are as defined above or R⁷ and R⁸ together form a 2-oxo-substituted 5- to 7-membered heterocyclyl ring,
   (e) —NR⁷SO₂R⁸ wherein R⁷ and R⁸ are as defined above or R⁷ and R⁸ together form a S,S-dioxo-substituted 5- to 7-membered heterocyclyl ring optionally substituted with one or two substituents independently selected from hydroxyl, (1-4C)alkyl, (1-4C)alkanoyl and methoxy;
   (f) —NR⁷R⁸ wherein R⁷ and R⁸ are as defined above or R⁷ and R⁸ together with the nitrogen to which they are attached form a 5- to 7-membered heterocyclyl ring optionally substituted with one or two substituents independently selected from hydroxyl, (1-4C)alkyl, (1-4C)alkanoyl and methoxy;
   (g) —OR⁷ wherein R⁷ is as defined above (excluding hydrogen);
   (h) —S(O)═NR¹¹ wherein R¹¹ is H or methyl;
      and wherein any carbon atom in a linear (1-3C)alkyl, (1-3C)alkyl or (1-2C)alkoxy containing group defined above may be optionally substituted by up to 3 fluoro atoms;
      with the proviso that:
(i) when q is 0 or r2 is 0 then Z cannot be hydroxyl or —OR⁷;
   or a pharmaceutically-acceptable salt, or pro-drug thereof.

According to a further aspect of the invention there is provided a compound of formula (I) or a pharmaceutically-acceptable salt, thereof, wherein:
Z is selected from hydrogen, fluoro, chloro and bromo, or is selected from one of the following eight groups:
   a) —CONR⁷R⁸ wherein R⁷ and R⁸ are independently selected from —(CR⁵R⁶)ᵤ(3-5C)cycloalkyl, —(CR⁵R⁶)ₛNR⁹R¹⁰, —(CR⁵R⁶)ₛ₁—O—(CR⁵R⁶)ₛ₂NR⁹R¹⁰, —(CR⁵R⁶)ᵥ-(4- to 7-membered heterocyclyl ring) and —(CR⁵R⁶)_{w}-(5- to 7-membered heteroaryl ring) or R⁷ and R⁸ together with the nitrogen to which they are attached form a 4- to 7-membered heterocyclic ring, 7- to 8-membered spirocyclic heterocyclic ring system, or 6- to 10-membered fused bicyclic heterocyclic ring system, wherein any ring or ring system is optionally substituted with one or two groups independently selected from oxo, hydroxyl, hydroxy(1-3C)alkyl, methoxy, amino, N-(1-3C)alkylamino and N,N-di(1-3C)alkylamino;
      provided that the 5- to 7-membered heteroaryl ring is not tetrazolyl;
      wherein:
      the alkyl, cycloalkyl and heterocyclyl are optionally substituted by hydroxyl, (1-4C)alkanoyl or methoxy, and the cycloalkyl and heterocyclyl are optionally substituted by (1-4C)alkyl; and the heteroaryl ring is optionally substituted by fluoro, chloro, cyano, methyl, trifluoromethyl or difluoromethyl;
      s, s1 and s2 are independently 2 or 3;
      u, v and w are independently 0, 1, 2 or 3;
      R⁵ and R⁶ are as defined above;
      R⁹ and R¹⁰ are independently selected from hydrogen, (1-3C)alkyl, (1-6C)alkoxycarbonyl, (3-5C)cycloalkyl and a 3- to 5-membered heterocyclyl ring, or R⁹ and R¹⁰ together with the nitrogen to which they are attached form a 4- to 7-membered heterocyclic ring optionally substituted with one or two groups independently selected from (1-4C)alkyl, hydroxy(1-4C)alkyl, oxo, (1-4C)alkanoyl, hydroxy and methoxy;
   (b) —SO₂NR^{7a}R^{8a}, wherein R^{7a} and R^{8a} are independently selected from hydrogen and variables defined above for R⁷ and R⁸;
   (c) —S(O)ₜR⁷, wherein R⁷ is as defined above (excluding hydrogen) and t is 0, 1 or 2;
   (d) —NR⁷COR⁸ wherein R⁷ and R⁸ are as defined above or R⁷ and R⁸ together form a 2-oxo-substituted 5- to 7-membered heterocyclyl ring;
   (e) —NR⁷SO₂R⁸ wherein R⁷ and R⁸ are as defined above or R⁷ and R⁸ together form a S,S-dioxo-substituted 5- to 7-membered heterocyclyl ring optionally substituted with one or two substituents independently selected from hydroxyl, (1-4C)alkyl, (1-4C)alkanoyl and methoxy;
   (f) —NR⁷R⁸ wherein R⁷ and R⁸ are as defined above or R⁷ and R⁸ together with the nitrogen to which they are attached form a 5- to 7-membered heterocyclyl ring optionally substituted with one or two substituents independently selected from hydroxyl, (1-4C)alkyl, (1-4C)alkanoyl and methoxy;
   (g) —OR⁷ wherein R⁷ is as defined above;
   (h) —S(O)═NR¹¹ wherein R¹¹ is H or methyl;
      or a pharmaceutically-acceptable salt, or pro-drug thereof.

According to a further aspect of the invention there is provided a compound of formula (I) or a pharmaceutically-acceptable salt, thereof, Z is selected from hydrogen, fluoro, chloro and bromo, or is selected from one of the following eight groups:
a) —CONR⁷R⁸ wherein R⁷ and R⁸ are independently selected from —(CR⁵R⁶)ᵤ(3-5C)cycloalkyl, —(CR⁵R⁶)ₛNR⁹R¹⁰, —(CR⁵R⁶)ₛ₁—O—(CR⁵R⁶)ₛ₂NR⁹R¹⁰ and —(CR⁵R⁶)ᵥ-(4- to 7-membered heterocyclyl ring),
   or R⁷ and R⁸ together with the nitrogen to which they are attached form a 4- to 7-membered heterocyclic ring, 7- to 8-membered spirocyclic heterocyclic ring system, or 6- to 10-membered fused bicyclic heterocyclic ring system, wherein any ring or ring system is optionally substituted with one or two groups independently selected from oxo, hydroxyl, hydroxy(1-3C)alkyl, methoxy, amino, N-(1-3C)alkylamino and N,N-di(1-3C)alkylamino;
   wherein:
   the alkyl, cycloalkyl and heterocyclyl are optionally substituted by hydroxyl, (1-4C)alkanoyl or methoxy, and the cycloalkyl and heterocyclyl are optionally substituted by (1-4C)alkyl; and the heteroaryl ring is optionally substituted by fluoro, chloro, cyano, methyl, trifluoromethyl or difluoromethyl;
   s, s1 and s2 are independently 2 or 3;
   u, v and w are independently 0, 1, 2 or 3;
   R⁵ and R⁶ are as defined above;
   R⁹ and R¹⁰ are independently selected from hydrogen, (1-3C)alkyl, (1-6C)alkoxycarbonyl, (3-5C)cycloalkyl and a 3- to 5-membered heterocyclyl ring, or R⁹ and R¹⁰ together with the nitrogen to which they are attached form a 4- to 7-membered heterocyclic ring optionally substituted with one or two groups independently selected from (1-4C)alkyl, hydroxy(1-4C)alkyl, oxo, (1-4C)alkanoyl, hydroxy and methoxy;
(b) —SO₂NR^{7a}R^{8a}, wherein R^{7a} and R^{8a} are independently selected from hydrogen and variables defined above for R⁷ and R⁸;
(c) —S(O)ₜR⁷, wherein R⁷ is as defined above (except hydrogen) and t is 0, 1 or 2;
(d) —NR⁷COR⁸ wherein R⁷ and R⁸ are as defined above or R⁷ and R⁸ together form a 2-oxo-substituted 5- to 7-membered heterocyclyl ring;
(e) —NR⁷SO₂R⁸ wherein R⁷ and R⁸ are as defined above or R⁷ and R⁸ together form a S,S-dioxo-substituted 5- to 7-membered heterocyclyl ring optionally substituted with one or two substituents independently selected from hydroxyl, (1-4C)alkyl, (1-4C)alkanoyl and methoxy;
(f) —NR⁷R⁸ wherein R⁷ and R⁸ are as defined above or R⁷ and R⁸ together with the nitrogen to which they are attached form a 5- to 7-membered heterocyclyl ring optionally substituted with one or two substituents independently selected from hydroxyl, (1-4C)alkyl, (1-4C)alkanoyl and methoxy;
(g) —OR⁷ wherein R⁷ is as defined above;
(h) —S(O)═NR¹¹ wherein R¹¹ is H or methyl;
   or a pharmaceutically-acceptable salt, or pro-drug thereof.

A further feature is any of the claims or embodiments herein with the proviso that any of the specific Examples herein, or a pharmaceutically-acceptable salt of any of these, are individually disclaimed.

It will be understood that reactive and/or unstable compounds are not included within the scope of the claims.

In this specification the term “alkyl”, for example (1-4C)alkyl, includes both straight and branched chain alkyl groups, unless otherwise stated, and references to individual alkyl groups such as “propyl” are specific for the straight chain version only. An analogous convention applies to other generic terms. Unless otherwise stated the term “alkyl” advantageously refers to chains with 1-10 carbon atoms, suitably from 1-6 carbon atoms, preferably 1-4 carbon atoms.

In this specification the term “alkoxy”, for example for example (1-4C)alkoxy, means an alkyl group as defined hereinbefore linked to an oxygen atom.

Particular values include for linear (1-3C)alkyl, methyl, ethyl and propyl; for (1-4C)alkyl, methyl, ethyl, propyl and butyl; for (2-3C)alkenyl, ethenyl; for (2-3C)alkynyl, ethynyl; for (1-2C)alkoxy, methoxy and ethoxy; for (1-4C)alkoxy, methoxy, ethoxy and propoxy; for (1-4C)alkanoyl, formyl, acetyl, butanoyl and propanoyl.

Particular values include for any carbon atom in a linear (1-3C)alkyl, (1-2C)alkoxy, (1-4C)alkyl or (1-4C)alkoxy group that may be optionally substituted by up to 3 fluoro atoms, a group such as, for example, trifluoromethyl, difluoromethyl, difluoromethoxy or trifluoromethoxy.

It will be understood that reference herein to any carbon atom in a linear (1-3C)alkyl, (1-3C)alkyl or (1-2C)alkoxy containing group being optionally substituted by up to 3 fluoro atoms means that any such linear (1-3C)alkyl, (1-3C)alkyl or (1-2C)alkoxy containing group may contain 0, 1, 2 or 3 fluoro atoms.

It will also be understood that when Q is —CH₂— and Z is hydrogen then Q-Z forms a methyl group. Optional fluoro substitution on this example of a (1-3C)alkyl group provides Q-Z as difluoromethyl or trifluoromethyl.

The term “heterocyclyl” or “heterocyclic ring” refers to a saturated or partially unsaturated monocyclic ring, said ring containing up to 5 heteroatoms independently selected from nitrogen, oxygen or sulphur (optionally oxidised to form S(O) or SO₂ groups), excluding O—O, S—S or O—S bonds and linked via ring carbon atoms or ring nitrogen atoms where a bond from a nitrogen is allowed. For example, a 4- to 7-membered heterocyclyl ring refers to a saturated or partially unsaturated ring containing between 4 and 7 atoms of which up to 5 atoms (where possible) are independently selected from nitrogen, oxygen or sulphur. Suitably, examples of 4- to 7-membered heterocyclic rings are 4-, 5- or 6-membered ring systems, particularly 5- or 6-membered ring systems, containing one or two ring heteroatoms. Examples of 4,5- or 6-membered saturated heterocyclic rings include azetidinyl, pyrrolidinyl, tetrahydrofuranyl, imidazolidinyl, piperidinyl, piperazinyl, morpholinyl and thiomorpholinyl. Examples of 3- to 5-membered heterocyclic rings are oxiranyl, aziridinyl, azetidinyl, oxetanyl, pyrrolinyl and tetrahydrofuryl.

The term “7- to 8-membered spirocyclic heterocyclic ring system” refers to a saturated system and includes ring systems such as:

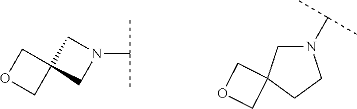

The term “6- to 10-membered fused bicyclic heterocyclic ring system” refers to a saturated fused bicyclic ring system, containing up to 3 heteroatoms (preferably 1 or 2 heteroatoms) independently selected from O, N and S (provided there are no O—O, S—S, or O—S bonds) and wherein at least one of the rings is heterocyclic. It will be understood that such rings are generally formed by NR⁷R⁸ and therefore must contain at least 1 ring nitrogen. Examples of such rings include:

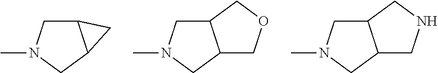

The term “5- to 7-membered heteroaryl ring” refers to a fully aromatic ring system containing 5, 6 or 7 ring atoms, of which up to 4 may be heteroatoms, selected from O, N and S (provided there are no O—O, S—S, or O—S bonds). Examples of such rings include pyridyl, pyrimidinyl, furyl, pyrazolyl, triazolyl, pyrazinyl, pyridazinyl, tetrazolyl, oxazolyl, isoxazolyl, thiazolyl and isothiazolyl.

For the avoidance of doubt it is to be understood that where in this specification a group is qualified by ‘hereinbefore defined’ or ‘defined hereinbefore’ the said group encompasses the first occurring and broadest definition as well as each and all of the particular definitions for that group.

If not stated elsewhere, suitable optional substituents for a particular group are those as stated for similar groups herein.

According to one embodiment, embodiment A, of the invention there is provided a compound of formula (I) wherein:
R¹ is selected from hydrogen, (1-4C)alkyl, (1-4C)alkoxy, (1-4C)perfluororoalkyl, and (1-4C)perfluororoalkoxy;
R² and R³ are independently selected from hydrogen, (1-4C)alkyl and (1-4C)perfluororoalkyl, or R² and R³ together with the carbon to which they are attached from a (3-6C)cycloalkyl ring;
R⁴ is selected from hydrogen and (1-4C)alkyl;
each q is independently 0 or 1 and each X₂ is independently selected from fluoro, chloro, bromo, amino, cyano, (1-3C)alkyl, (2-3C)alkenyl, (2-3C)alkynyl and (1-2C)alkoxy;
Y₁ is selected from fluoro, chloro, bromo, cyano, (1-3C)alkyl and (1-2C)alkoxy;
n is 0, 1 or 2 and each Y₂ is independently selected from fluoro, chloro, bromo, cyano, hydroxy, (1-3C)alkyl and (1-2C)alkoxy;
Q is selected from a direct bond, —(CR⁵R⁶)ₚ—, —O—(CR⁵R⁶)_{q}— and —(CR⁵R⁶)ᵣ₁—O—(CR⁵R⁶)ᵣ₂—wherein p is 1, 2 or 3, q is 0, 1 or 2 and r1 and r2 are independently 0 or 1 and R⁵ and R⁶ are independently selected from hydrogen, methyl and ethyl;
Z is hydroxy or is selected from one of the following seven groups:
   (a) —CONR⁷R⁸ wherein R⁷ and R⁸ are independently selected from hydrogen, (C1-3)alkyl, (C3-5)cycloalkyl, —(CR⁵R⁶)ₛNR⁹R¹⁰ and (C3-5)heterocyclyl or R⁷ and R⁸ together with the nitrogen to which they are attached form a (C4-7)heterocyclic ring optionally substituted with one or two oxo, hydroxy or methoxy groups or R⁹ and R¹⁰ together with the nitrogen to which they are attached form a (C4-7)heterocyclic ring optionally substituted with one or two oxo, hydroxy or methoxy groups;
      wherein:
      the alkyl, cycloalkyl and heterocyclyl are optionally substituted by hydroxy or methoxy;
      s is 2 or 3;
      R⁵ and R⁶ are as defined above;
      R⁹ and R¹⁰ are independently selected from hydrogen, (C1-3)alkyl, (C3-5)cycloalkyl and (C3-5)heterocyclyl wherein R⁵ and R⁶ are as defined above;
   (b) —SO₂NR^{7a}R^{8a}, wherein R^{7a} and R^{8a} are independently selected from hydrogen and variables defined above for R⁷ and R⁸;
   (c) —S(O)ₜR⁷, wherein R⁷ is as defined above and t is 0, 1 or 2;
   (d) —NR⁷COR⁸ wherein R⁷ and R⁸ are as defined above or R⁷ and R⁸ together form a 2-oxo(C5-7)heterocyclyl;
   (e) —NR⁷SO₂R⁸ wherein R⁷ and R⁸ are as defined above or R⁷ and R⁸ together form a S,S-dioxo (C5-7)heterocyclyl optionally substituted with one or two hydroxy or methoxy groups;
   (f) —NR⁷R⁸ wherein R⁷ and R⁸ are as defined above or R⁷ and R⁸ together with the nitrogen to which they are attached form a (C5-7)heterocyclyl optionally substituted with one or two hydroxy or methoxy groups;
   (g) —OR⁷ wherein R⁷ is as defined above;
      and wherein any carbon atom in a linear (1-3C)alkyl, (1-3C)alkyl or (1-2C)alkoxy containing group defined above may be optionally substituted by up to 3 fluoro atoms;
      with the proviso that:
   i) when q is 0 or r2 is 0 then Z cannot be hydroxyl or —OR⁷;
      or a pharmaceutically-acceptable salt, or pro-drug thereof.

According to a further aspect of embodiment A of the invention there is provided a compound of formula (I) or a pharmaceutically-acceptable salt, thereof, wherein Z is selected from one of the following seven groups:
(a) —CONR⁷R⁸ wherein R⁷ and R⁸ are independently selected from (C3-5)cycloalkyl, —(CR⁵R⁶)ₛNR⁹R¹⁰ and (C3-5)heterocyclyl or R⁷ and R⁸ together with the nitrogen to which they are attached form a (C4-7)heterocyclic ring optionally substituted with one or two oxo, hydroxy or methoxy groups or R⁹ and R¹⁰ together with the nitrogen to which they are attached form a (C4-7)heterocyclic ring optionally substituted with one or two oxo, hydroxy or methoxy groups;
   with the proviso that the (C3-5)heterocyclyl group cannot be tetrazolyl;
   wherein:
   the alkyl, cycloalkyl and heterocyclyl are optionally substituted by hydroxy or methoxy;
   s is 2 or 3;
   R⁵ and R⁶ are as defined above;
   R⁹ and R¹⁰ are independently selected from hydrogen, (C1-3)alkyl, (C3-5)cycloalkyl and (C3-5)heterocyclyl wherein R⁵ and R⁶ are as defined above;
(b) —SO₂NR^{7a}R^{8a}, wherein R^{7a} and R^{8a} are independently selected from hydrogen and variables defined above for R⁷ and R⁸;
(c) —S(O)ₜR⁷, wherein R⁷ is as defined above and t is 0, 1 or 2;
(d) —NR⁷COR⁸ wherein R⁷ and R⁸ are as defined above or R⁷ and R⁸ together form a 2-oxo (C5-7)heterocyclyl;
(e) —NR⁷SO₂R⁸ wherein R⁷ and R⁸ are as defined above or R⁷ and R⁸ together form a S,S-dioxo (C5-7)heterocyclyl optionally substituted with one or two hydroxy or methoxy groups;
(f) —NR⁷R⁸ wherein R⁷ and R⁸ are as defined above or R⁷ and R⁸ together with the nitrogen to which they are attached form a (C5-7)heterocyclyl optionally substituted with one or two hydroxy or methoxy groups;
(g) —OR⁷ wherein R⁷ is as defined above;
   or a pharmaceutically-acceptable salt, or pro-drug thereof.

According to a further aspect of embodiment A of the invention there is provided a compound of formula (I) or a pharmaceutically-acceptable salt, thereof, wherein Z is selected from one of the following seven groups:
(a) —CONR⁷R⁸ wherein R⁷ and R⁸ are independently selected from (C3-5)cycloalkyl, and —(CR⁵R⁶)ₛNR⁹R¹⁰ or R⁷ and R⁸ together with the nitrogen to which they are attached form a (C4-7)heterocyclic ring optionally substituted on with one or two oxo, hydroxy or methoxy groups or R⁹ and R¹⁰ together with the nitrogen to which they are attached form a (C4-7)heterocyclic ring optionally substituted with one or two oxo, hydroxy or methoxy groups;
   the alkyl and cycloalkyl are optionally substituted by hydroxy or methoxy;
   s is 2 or 3;
   R⁵ and R⁶ are as defined above;
   R⁹ and R¹⁰ are independently selected from hydrogen, (C1-3)alkyl, (C3-5)cycloalkyl and (C3-5)heterocyclyl wherein R⁵ and R⁶ are as defined above;
(b) —SO₂NR^{7a}R^{8a}, wherein R^{7a} and R^{8a} are independently selected from hydrogen and variables defined above for R⁷ and R⁸;
(c) —S(O)ₜR⁷, wherein R⁷ is as defined above and t is 0, 1 or 2;
(d) —NR⁷COR⁸ wherein R⁷ and R⁸ are as defined above or R⁷ and R⁸ together form a 2-oxo (C5-7)heterocyclyl;
(e) —NR⁷SO₂R⁸ wherein R⁷ and R⁸ are as defined above or R⁷ and R⁸ together form a S,S-dioxo (C5-7)heterocyclyl optionally substituted with one or two hydroxy or methoxy groups;
(f) —NR⁷R⁸ wherein R⁷ and R⁸ are as defined above or R⁷ and R⁸ together with the nitrogen to which they are attached form a (C5-7)heterocyclyl optionally substituted with one or two hydroxy or methoxy groups;
(g) —OR⁷ wherein R⁷ is as defined above;
   or a pharmaceutically-acceptable salt, or pro-drug thereof.

In the above embodiment A (and aspects thereof), the term “heterocyclyl” or “heterocyclic ring” refers to a saturated mono or bicyclic ring, said saturated ring containing up to 5 heteroatoms independently selected from nitrogen, oxygen or sulphur, linked via ring carbon atoms or ring nitrogen atoms where a bond from a nitrogen is allowed. For example, (C4-7)heterocyclyl refers to a saturated ring containing between 4 to 7 atoms of which up to 5 atoms are independently selected from nitrogen, oxygen or sulphur. Examples of 4,5- or 6-membered saturated heterocyclic rings include azetidinyl, pyrrolinyl, tetrahydrofuranyl, imidazolidinyl, piperidinyl, piperazinyl, morpholinyl and thiomorpholinyl. This definition further comprises sulphur-containing rings wherein the sulphur atom has been oxidised to an S(O) or S(O)₂ group.

A compound of formula (I) may form stable acid or basic salts, and in such cases administration of a compound as a salt may be appropriate, and pharmaceutically acceptable salts may be made by conventional methods such as those described following.

Suitable pharmaceutically-acceptable salts include acid addition salts such as methanesulfonate, tosylate, α-glycerophosphate, fumarate, hydrochloride, citrate, maleate, tartrate and (less preferably) hydrobromide. Also suitable are salts formed with phosphoric and sulfuric acid. Further suitable pharmaceutically-acceptable salts include acetate, aspartate, benzoate, besylate, edisylate, esylate, hemifumarate, lactate, malate, napsylate, saccharate, stearate, succinate, or trifluoroacetate salt. There may be more than one cation or anion depending on the number of charged functions and the valency of the cations or anions.

Other suitable pharmaceutically-acceptable salts are mentioned in, for example, Berge et al. (J. Pharm. Sci., 1977, 66, 1-19) and/or Handbook of Pharmaceutical Salts: Properties, Selection and Use by Stahl and Wermuth (Wiley-VCH, 2002).

A feature of the invention relates to a compound of the invention, such as any one of the Examples, in the free acid or free base form or as a pharmaceutically acceptable salt thereof. Such forms may be prepared by standard techniques.

However, to facilitate isolation of the salt during preparation, salts which are less soluble in the chosen solvent may be preferred whether pharmaceutically-acceptable or not. Such salt comprise a further embodiment of the invention.

Within the present invention it is to be understood that a compound of the formula (I) or a salt thereof may exhibit the phenomenon of tautomerism and that the formulae drawings within this specification can represent only one of the possible tautomeric forms. It is to be understood that the invention encompasses any tautomeric form which inhibits DGAT1 activity and is not to be limited merely to any one tautomeric form utilised within the formulae drawings.

Pro-drugs of compounds of formula (I), and salts thereof, are also within the scope of the invention.

Various forms of prodrugs are known in the art. For examples of such prodrug derivatives, see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

Examples of such prodrugs are in vivo cleavable esters of a compound of the invention. An in vivo cleavable ester of a compound of the invention containing a hydroxy group is, for example, a pharmaceutically-acceptable ester which is cleaved in the human or animal body to produce the parent hydroxy group. Suitable pharmaceutically acceptable esters for hydroxy include (1-6C)alkanoyl esters, for example acetyl esters; and benzoyl esters wherein the phenyl group may be substituted with aminomethyl or N-substituted mono- or di-(1-6C)alkyl aminomethyl, for example 4-aminomethylbenzoyl esters and 4-N,N-dimethylaminomethylbenzoyl esters.

It will be appreciated by those skilled in the art that certain compounds of formula (I) contain asymmetrically substituted carbon and/or sulfur atoms, and accordingly may exist in, and be isolated in, optically-active and racemic forms. Some compounds of formula (I) may exhibit polymorphism. It is to be understood that the present invention encompasses any racemic, optically-active, polymorphic or stereoisomeric form, or mixtures thereof, which form possesses properties useful in the inhibition of DGAT1 activity, it being well known in the art how to prepare optically-active forms (for example, by resolution of the racemic form by recrystallization techniques, by synthesis from optically-active starting materials, by chiral synthesis, by enzymatic resolution, by biotransformation, or by chromatographic separation using a chiral stationary phase) and how to determine efficacy for the inhibition of DGAT1 activity by the standard tests described hereinafter.

It is also to be understood that certain compounds of the formula (I) and salts thereof can exist in solvated as well as unsolvated forms such as, for example, hydrated forms. It is to be understood that the invention encompasses all such solvated forms which inhibit DGAT1 activity.

The present invention is intended to include all isotopes of atoms occurring in the present compounds. Isotopes will be understood to include those atoms having the same atomic number but different mass numbers. For example, isotopes of hydrogen include tritium and deuterium. Isotopes of carbon include ¹³C and ¹⁴C.

As stated before, a range of compounds are provided that have good DGAT1 inhibitory activity. They have good physical and/or pharmacokinetic properties in general. The following compounds possess particular, desirable pharmaceutical and/or physical and/or pharmacokinetic/dynamic and/or toxicological properties and/or selective activity for DGAT1.

In one embodiment there is provided a compound as claimed in any one of the claims, or a pharmaceutically-acceptable salt, or pro-drug thereof, wherein the pyrazine is substituted on an available carbon atom by one or two linear (1-3C)alkyl substituents, in particular methyl, and in particular dimethyl.

Particular values of substituents in compounds of formula (I) are as follows (such values may be used where appropriate with any of the other values, definitions, claims or embodiments defined hereinbefore or hereinafter),
(1) R¹ is hydrogen or (1-4C)alkyl;
(2) R¹ is hydrogen;
(3) R¹ is methyl or ethyl, for example methyl;
(4) R² is hydrogen or (1-4C)alkyl;
(5) R² is hydrogen;
(6) R² is methyl or ethyl, for example methyl;
(7) R³ is hydrogen or (1-4C)alkyl;
(8) R³ is hydrogen;
(9) R³ is methyl or ethyl, for example methyl;
(10) R² is hydrogen and R³ is (1-4C)alkyl;
(11) R² and R³ are both hydrogen;
(12) R⁴ is hydrogen, methyl or ethyl;
(13) R⁴ is hydrogen;
(14) R¹, R², R³ and R⁴ are all hydrogen;
(15) R¹ is methyl and R², R³ and R⁴ are all hydrogen;
(16) R² is (C1-3)alkyl, for example methyl or ethyl and R¹, R³ and R⁴ are all hydrogen;
(17) R² is methyl, for example R-methyl or S-methyl and R¹, R³ and R⁴ are all hydrogen;
(18) R³ is methyl, for example R-methyl or S-methyl and R¹, R² and R⁴ are all hydrogen;
(19) R⁴ is methyl, for example S-methyl or R-methyl, and R¹, R³ and R³ are all hydrogen;
(20) R² and R³ are both methyl and R¹ and R⁴ are both hydrogen;
(21) R² and R³ together with the carbon to which they are attached form cyclopropyl;
(22) R² and R³ together with the carbon to which they are attached form cyclobutyl;
(23) R² and R³ together with the carbon to which they are attached form cyclopentyl;
(24) R² and R³ together with the carbon to which they are attached form cyclohexyl;
(25) q is 0 or 1;
(26) one q=1 and one q=0;
(27) both q=0
(28) X₂ is fluoro or chloro;
(29) X₂ is fluoro or cyano;
(30) X₂ is fluoro
(31) one q=1 and one q=0, X₂ is fluoro, chloro or cyano (such as fluoro or cyano)
(32) Y₁ is selected from fluoro, chloro, bromo, cyano, (1-3C)alkyl and (1-2C)alkoxy;
(33) Y₁ is fluoro, chloro, cyano, methyl or trifluoromethyl;
(34) Y₁ is fluoro, chloro, methyl or trifluoromethyl;
(35) Y₁ is fluoro, chloro or bromo, for example chloro;
(36) Y₁ is fluoro, chloro or methyl;
(37) Y₁ is fluoro, chloro or bromo;
(38) Y₁ is chloro;
(39) n is 0 or 1;
(40) q is 0;
(41) Y₂ is fluoro, chloro or (1-3C)alkyl;
(42) Y₂ is fluoro, chloro, cyano or (1-3C)alkyl;
(43) Y₂ is fluoro, chloro or methyl;
(44) Y₂ is fluoro or chloro;
(45) Both q are 0, Y₁ is fluoro, chloro or bromo, for example chloro or fluoro and n is 0;
(46) Both q are 0, Y₁ is chloro and q is 0;
(47) one q=1 and one q=0, X₂ is fluoro, chloro or cyano (such as fluoro or cyano), Y₁ is chloro and n is 0
(48) Both q are 0, Z is H, n is 1, Y₁ and Y₂ are each either chloro or fluoro;
(49) Q is —(CR⁵R⁶)ₚ—, for example where p=1;
(50) Q is —CH₂—;
(51) Q is a direct bond;
(52) Q is —CH₂— or a direct bond
(53) Q is —CH₂— or a direct bond and Z is H, fluoro or chloro (provided Q is a direct bond if Z is chloro)
(54) Z is selected from hydrogen, hydroxyl, fluoro, chloro, bromo, cyano, difluoromethyl and trifluoromethyl
(55) In embodiment A, Z is —CONR⁷R⁸ wherein R⁷ and R⁸ together with the nitrogen to which they are attached form a (C4-7)heterocyclic ring optionally substituted with one or two oxo, hydroxy or methoxy groups;
(56) Z is —CONR⁷R⁸ wherein R⁷ and R⁸ together with the nitrogen to which they are attached form a 4- to 7-membered heterocyclic ring optionally substituted with one or two substituents independently selected from oxo, hydroxy and methoxy;
(57) Z is —CONR⁷R⁸ wherein R⁷ and R⁸ are independently selected from (1-3C)alkyl, (3-5C)cycloalkyl, —(CR⁵R⁶)ₛNR⁹R¹⁰ and a heterocyclyl ring selected from oxetanyl, tetrahydrofuryl, tetrahydropyranyl and azetidinyl and wherein the heterocyclyl ring is optionally substituted by (1-3C)alkyl;
(58) Z is —CONR⁷R⁸ wherein R⁷ and R⁸ are independently selected from (1-3C)alkyl, (3-5C)cycloalkyl, —(CR⁵R⁶)ₛNR⁹R¹⁰ and a heterocyclyl ring selected from oxetan-3-yl, tetrahydro-3-furyl, tetrahydropyran-4-yl, azetidin-3-yl and 1-methylazetidin-3-yl;
(59) R⁷ and R⁸ together with the nitrogen to which they are attached to form a heterocyclic ring optionally substituted on with one or two oxo, hydroxy or methoxy groups wherein the heterocyclic ring is selected from azetidine, pyrrolidine, piperidine and piperazine;
(60) Z is NR⁷COR⁸ wherein R⁷ and R⁸ are as defined above or R⁷ and R⁸ together form a heterocyclyl ring selected from pyrrolidinone, morpholinone and pyridinone;
(61) any carbon atom in a linear (1-3C)alkyl, (1-3C)alkyl or (1-2C)alkoxy containing group in X₂, Y₂ or Y₁ may be optionally substituted by up to 3 fluoro atoms;
(62) R¹, R², R³ and R⁴ are all hydrogen, both q=0, n=0, Y₁ is fluoro, chloro or bromo, such as fluoro or chloro, for example chloro, Q is —CH₂— or a direct bond, Z is or —CONR⁷R⁸ or —SO₂NR^{7a}R^{8a}, wherein R⁷ and R⁸ together with the nitrogen to which they are attached form a (C4-7)heterocyclic ring optionally substituted on with oxo or hydroxy and R^{7a} and R^{8a} are both hydrogen or together with the nitrogen to which they are attached form a (C4-7)heterocyclic ring optionally substituted with oxo or hydroxyl;
(63) R¹, R², R³ and R⁴ are all hydrogen, both q=0, n=0, Y₁ is fluoro, chloro or bromo, such as fluoro or chloro, for example chloro, Q is —CH₂— or a direct bond, Z is or —CONR⁷R⁸ or —SO₂NR^{7a}R^{8a}, wherein R⁷ and R⁸ together with the nitrogen to which they are attached form a 4- to 7-membered heterocyclic ring optionally substituted on with oxo or hydroxyl, and R^{7a} and R^{8a} are both hydrogen or together with the nitrogen to which they are attached form a 4- to 7-membered heterocyclic ring optionally substituted with oxo or hydroxyl;
(64) R¹, R², R³ and R⁴ are all hydrogen, both q=0, n=0, Y₁ is fluoro, chloro or bromo, such as fluoro or chloro, for example chloro, Q is —CH₂— or a direct bond, Z is or —CONR⁷R⁸ or —SO₂NR^{7a}R^{8a}, wherein R⁷ and R⁸ together with the nitrogen to which they are attached form a (C4-7)heterocyclic ring optionally substituted on with oxo or hydroxy and R^{7a} and R^{8a} are both hydrogen or together with the nitrogen to which they are attached form a (C4-7)heterocyclic ring selected from pyrrolidinyl, piperazinyl and azetidinyl optionally substituted with oxo or hydroxyl;
(65) R¹, R², R³ and R⁴ are all hydrogen, both q=0, n=0, Y₁ is fluoro, chloro or bromo, such as fluoro or chloro, for example chloro, Q is —CH₂— or a direct bond, Z is or —CONR⁷R⁸ or —SO₂NR^{7a}R^{8a}, wherein R⁷ and R⁸ together with the nitrogen to which they are attached form a 4- to 7-membered heterocyclic ring optionally substituted on with oxo or hydroxyl, and R^{7a} and R^{8a} are both hydrogen or together with the nitrogen to which they are attached form a heterocyclic ring selected from pyrrolidinyl, piperazinyl and azetidinyl optionally substituted with oxo or hydroxyl;
(66) R¹, R², R³ and R⁴ are all hydrogen, both q=0, n=0, Y₁ is fluoro, chloro or bromo, such as fluoro or chloro, for example chloro, Q is —CH₂—, Z is —CONR⁷R⁸, wherein R⁷ and R⁸ together with the nitrogen to which they are attached form a (C4-7)heterocyclic ring optionally substituted with oxo or hydroxy;
(67) R¹, R², R³ and R⁴ are all hydrogen, both q=0, n=0, Y₁ is fluoro, chloro or bromo, such as fluoro or chloro, for example chloro, Q is —CH₂—, Z is —CONR⁷R⁸, wherein R⁷ and R⁸ together with the nitrogen to which they are attached form a 4- to 7-membered heterocyclic ring optionally substituted with oxo or hydroxy;
(68) R¹, R², R³ and R⁴ are all hydrogen, both q=0, n=0, Y₁ is fluoro, chloro or bromo, such as fluoro or chloro, for example chloro, Q is —CH₂—, Z is —CONR⁷R⁸, wherein R⁷ and R⁸ together with the nitrogen to which they are attached form a heterocyclic ring selected from pyrrolidinyl, piperazinyl and azetidinyl optionally substituted with oxo or hydroxy;
(69) R¹, R², R³ and R⁴ are all hydrogen, both q=0, n=0, Y₁ is fluoro, chloro or bromo, such as fluoro or chloro, for example chloro, Q is a direct bond, Z is —SO₂NR^{7a}R^{8a}, wherein R^{7a} and R^{8a} are both hydrogen or together with the nitrogen to which they are attached form a (C4-7)heterocyclic ring optionally substituted with oxo or hydroxyl;
(70) R¹, R², R³ and R⁴ are all hydrogen, both q=0, n=0, Y₁ is fluoro, chloro or bromo, such as fluoro or chloro, for example chloro, Q is a direct bond, Z is —SO₂NR^{7a}R^{8a}, wherein R^{7a} and R^{8a} are both hydrogen or together with the nitrogen to which they are attached form a 4- to 7-membered heterocyclic ring optionally substituted with oxo or hydroxyl;
(71) R¹, R², R³ and R⁴ are all hydrogen, both q=0, n=0, Y₁ is fluoro, chloro or bromo, such as fluoro or chloro, for example chloro, Q is a direct bond, Z is —SO₂NR^{7a}R^{8a}, wherein R^{7a} and R^{8a} are both hydrogen.
(72) R¹, R², R³ and R⁴ are all hydrogen;
   each q is independently 0 or 1 and each X₂ is independently selected from fluoro and chloro; Y₁ is selected from fluoro, chloro and (1-3C)alkyl,
   n is 0 or 1 and each Y₂ is independently selected from fluoro, chloro and (1-3C)alkyl;
   Q is —(CR⁵R⁶)ₚ— and Z is a group (a);
(73) R¹, R², R³ and R⁴ are all hydrogen;
   each q is 0;
   Y₁ is selected from fluoro, chloro and (1-3C)alkyl (particularly methyl);
   n is 0 or 1 and Y₂ is independently selected from fluoro, chloro and (1-3C)alkyl (particularly methyl);
   Z is selected from hydrogen, fluoro, chloro and cyano;
   Q is a direct bond or —CH₂—;
(74) R¹, R², R³ and R⁴ are all hydrogen;
   each q is 0;
   Y₁ is selected from hydrogen, fluoro, chloro, cyano and (1-3C)alkyl (particularly methyl);
   n is 0 or 1 and Y₂ is independently selected from fluoro, chloro and (1-3C)alkyl (particularly methyl);
   Z is selected from hydrogen, fluoro, chloro, SO₂Me, CONR⁷R⁸ (wherein R⁷ and R⁸ together with the nitrogen to which they are attached form a heterocyclic ring selected from pyrrolidinyl, piperazinyl and azetidinyl optionally substituted with oxo or hydroxyl), SO₂NR^{7a}R^{8a}, (wherein R^{7a} and R^{8a} are both hydrogen), NR⁷SO₂R⁸ (wherein R⁷ is H or methyl and R⁸ is methyl) and NR⁷COR⁸ (wherein R⁷ and R⁸ are both methyl);
   Q is a direct bond or —CH₂—
(75) R¹, R³ and R⁴ are all hydrogen; R² is hydrogen or methyl;
   each q is 0 or 1;
   X² is selected from fluoro, chloro and cyano;
   Y₁ is selected from hydrogen, fluoro, chloro, cyano and (1-3C)alkyl (particularly methyl);
   n is 0 or 1 and Y₂ is independently selected from fluoro, chloro and (1-3C)alkyl (particularly methyl);
   Z is selected from hydrogen, hydroxyl, fluoro, chloro, cyano, difluoromethyl, —SO₂Me,
   —CONR⁷R⁸ (wherein R⁷ and R⁸ together with the nitrogen to which they are attached form a heterocyclic ring selected from pyrrolidinyl, piperazinyl and azetidinyl optionally substituted with oxo or hydroxyl),
   —SO₂NR^{7a}R^{8a}, (wherein R^{7a} and R^{8a} are both hydrogen),
   —NR⁷SO₂R⁸ (wherein R⁷ is H or methyl and R⁸ is methyl),
   and NR⁷COR⁸ (wherein R⁷ is hydrogen or methyl and R⁸ is selected from methyl, aminomethyl, aminoethyl and aminopropyl, or R⁷ and R⁸ together form a 2-oxo substituted 5- or 6-membered heterocyclic ring, optionally further substituted by hydroxy).

Surprisingly, we have found that compounds of formula (I) in which Q and Z together constitute a simple small substituent (including where Q and Z together are hydrogen) are potent inhibitors of DGAT-1. Therefore in a further aspect of the invention, there is provided a compound of formula (I) or a pharmaceutically-acceptable salt, or pro-drug thereof (particularly a compound or pharmaceutically-acceptable salt thereof)

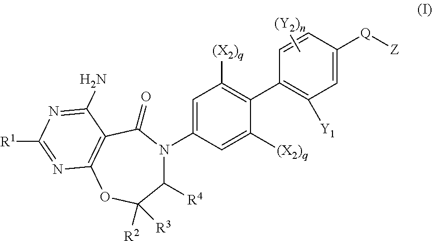

is wherein R¹, R², R³ and R⁴ are all hydrogen;
each q is 0;
Y₁ is selected from fluoro, chloro and (1-3C)alkyl;
n is 0 or 1 and Y₂ is selected from fluoro, chloro and (1-3C)alkyl;
Z is selected from hydrogen, fluoro, chloro, difluoromethyl and cyano;
Q is a direct bond or —CH₂—.

In a still further aspect of the invention, there is provided a compound of formula (I) or a pharmaceutically-acceptable salt, or pro-drug thereof (particularly a compound or pharmaceutically-acceptable salt thereof),

wherein R¹, R², R³ and R⁴ are all hydrogen;
each q is 0;
Y₁ is selected from fluoro, chloro and (1-3C)alkyl;
n is 0 or 1 and Y₂ is selected from fluoro, chloro and (1-3C)alkyl;
Z is selected from hydrogen, fluoro, chloro and cyano;
Q is a direct bond or —CH₂—.

In a still further aspect of the invention, there is provided a compound of formula (I) or a pharmaceutically-acceptable salt, or pro-drug thereof (particularly a compound or pharmaceutically-acceptable salt thereof),

wherein R¹, R², R³ and R⁴ are all hydrogen;
each q is 0;
Y₁ is selected from fluoro, chloro and (1-3C)alkyl;
n is 0 or 1 and Y₂ is selected from fluoro, chloro and (1-3C)alkyl;
Q-Z is selected from hydrogen, methyl, difluoromethyl, fluoromethyl, fluoro, chloro and cyano.

In a still further aspect of the invention, there is provided a compound of formula (I) or a pharmaceutically-acceptable salt, or pro-drug thereof (particularly a compound or pharmaceutically-acceptable salt thereof),

wherein R¹, R³ and R⁴ are all hydrogen;
R² is hydrogen or methyl;
one q=1 and the other q=0, or both q=0;
X₂ is fluoro or cyano
Y₁ is selected from fluoro, chloro and (1-3C)alkyl;
n is 0 or 1 and Y₂ is selected from fluoro, chloro and (1-3C)alkyl;
Q-Z is selected from hydrogen, methyl, difluoromethyl, fluoromethyl, fluoro, chloro and cyano.

In a still further aspect of the invention, there is provided a compound of formula (I) or a pharmaceutically-acceptable salt, or pro-drug thereof (particularly a compound or pharmaceutically-acceptable salt thereof),

wherein R¹, R³ and R⁴ are all hydrogen;
R² is hydrogen or methyl;
one q=1 and the other q=0;
X₂ is fluoro or cyano
Y₁ is selected from fluoro, chloro and (1-3C)alkyl;
n is 0 or 1 and Y₂ is selected from fluoro, chloro and (1-3C)alkyl;
Q-Z is selected from hydrogen, methyl, difluoromethyl, fluoromethyl, fluoro, chloro and cyano.

In a still further aspect of the invention, there is provided a compound of formula (I) or a pharmaceutically-acceptable salt, or pro-drug thereof (particularly a compound or pharmaceutically-acceptable salt thereof),

wherein R¹, R³ and R⁴ are all hydrogen;
R² is hydrogen or methyl;
one q=1 and the other q=0;
X₂ is fluoro or cyano
Y₁ is fluoro;
n is 0 or 1 and Y₂ is selected from fluoro, chloro and (1-3C)alkyl;
Q-Z is selected from hydrogen, methyl, difluoromethyl, fluoromethyl, fluoro, chloro and cyano.

In a yet further aspect of the invention, there is provided a compound of formula (I) or a pharmaceutically-acceptable salt, or pro-drug thereof (particularly a compound or pharmaceutically-acceptable salt thereof),

wherein R¹, R³ and R⁴ are all hydrogen;
R² is hydrogen or methyl;
one q=1 and the other q=0;
X₂ is fluoro or cyano
Y₁ is chloro;
n is 0 or 1 and Y₂ is selected from fluoro, chloro and (1-3C)alkyl;
Q-Z is hydrogen, methyl, fluoro or chloro.

It will be understood that when R² is methyl and R³ is hydrogen, then a compound of formula (I) has a chiral centre. In one aspect the preferred stereochemistry is (R)-methyl as shown as formula (IA) below. In another aspect, the preferred stereochemistry is (S)-methyl as shown in figure (IB) below.

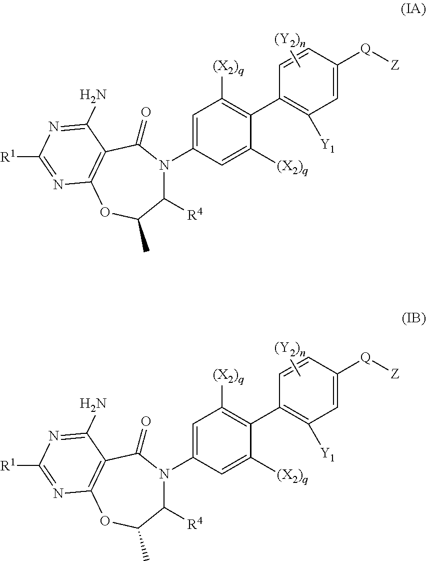

It will be further understood that the aspects, embodiments and preferred values mentioned herein in relation to compounds of formula (I) may also apply analogously to formula (IA) and (IB).

A further feature is any of the scopes defined herein with the proviso that any one or more of the specific Examples, such as Example 1, 2, 3, 4 etc. are individually disclaimed.

Further particular compounds of the invention are each of the Examples, each of which provides a further independent aspect of the invention. In further aspects, the present invention also comprises any particular compounds of the Examples or a pharmaceutically-acceptable salt thereof.

In a further aspect, the present invention also comprises any particular isomers of compounds of the Examples, or a pharmaceutically-acceptable salt of any of these.

In a further aspect, the present invention also comprises any groups of Examples falling within the scope of particular aspects of the invention as herein described, such as those of formula IA or IB, or a group comprising Examples 1 to 4, or a group comprising Examples 5 to 24, or a group comprising Examples 25 to 59, or pharmaceutically-acceptable salts of any of the Examples in these groups. A further group of the examples is Examples 50 to 59, or pharmaceutically-acceptable salts thereof. A further aspect of the invention comprises pro-drugs of compounds of any of the groups mentioned above.

A compound of formula (I) and its salts may be prepared by any process known to be applicable to the preparation of chemically related compounds. Such processes, when used to prepare a compound of the formula (I), or a pharmaceutically-acceptable salt thereof, are provided as a further feature of the invention.

In a further aspect the present invention also provides that the compounds of the formula (I) and salts thereof, can be prepared by the following processes (processes A, B (B1 to B4) and/or C), the processes of the Examples and analogous processes (wherein all variables are as hereinbefore defined for a compound of formula (I) unless otherwise stated) and thereafter if necessary any protecting groups can be removed and/or an appropriate salt formed.

Variables shown in the schemes are defined or can be interpreted in the context of the variants described herein for the compounds of the invention. Analogous chemistry to that shown in the schemes and Examples may be used to prepare other ring variants and linking group options within the scope of the invention.

Also included as an aspect of the invention are the compounds and intermediates obtainable by any of the processes or Examples described herein.

### Process A

By modifying a substituent in, or introducing a substituent into, another compound of formula (I). Suitable methods for converting substituents into other substituents are known in the art; for example, a sulphur atom can be oxidised to a sulfinyl or sulfonyl group.

### Process B

As described in the following processes (wherein the variables are appropriately as defined in any of the claims, embodiments or Examples herein), Suzuki coupling of an appropriate trifluoromethanesulphonyl, iodo-, bromo- or chloro-substituted aromatic compound can be performed with a suitably substituted intermediate boron-containing compound using standard methods with a suitable palladium catalyst, such as 1,1′-bis(diphenylphosphino)-ferrocenedichloro-palladium(II).

### Process B1

Compounds of the invention can be prepared using Suzuki couplings of a compounds of formula (II) with a compound of formula (III) (wherein X is trifluoromethanesulfonyl, iodo-, bromo- or chloro and wherein E represents a boronic acid (—B(OH)₂), a boronate ester (—B(OR)₂ wherein R here is (1-4C)alkyl) or a cyclic boronic ester, such as pinacolato borane), as illustrated by the scheme below.

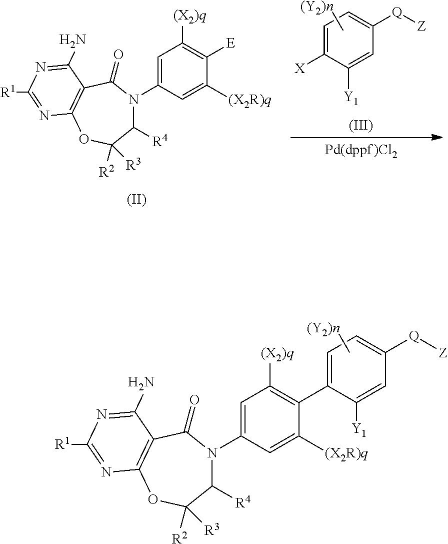

Compounds of Formula (II) represent a further embodiment of the invention and can be prepared by processes available to the skilled man, for example as outlined in Scheme B1-A below.

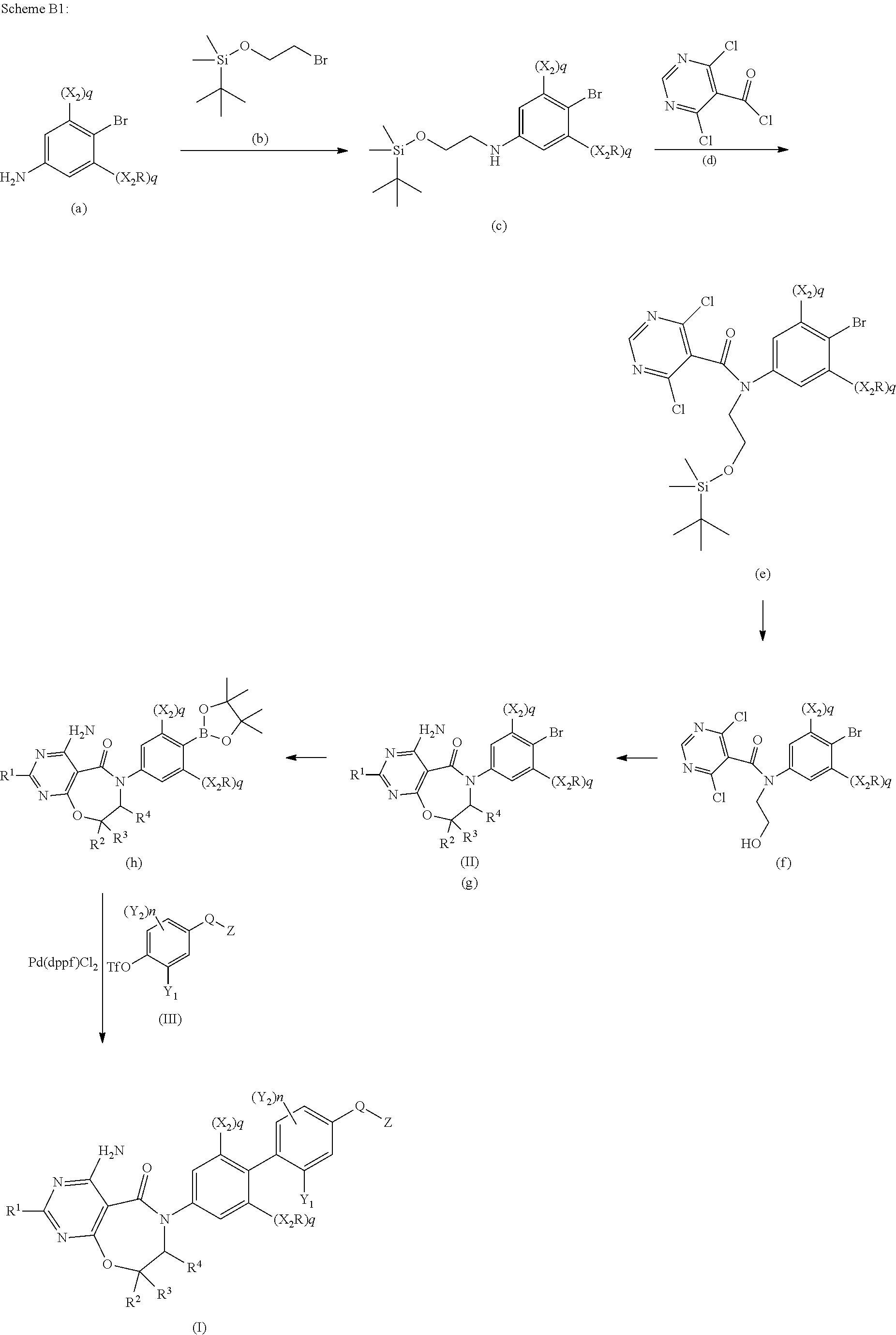

In this Scheme compounds of formula (a) can be prepared by processes well known to the skilled person. Compounds of formula (a) are reacted with compounds of formula (b) which can be prepared by the method of C. Palomo et al., Synthesis of β-Lactam Scaffolds for Ditopic Peptidomimetics, Organic Letters (2007), 9(1), pages 101-104. Compounds (a) and (b) can be reacted using a metal catalyst, such as palladium or copper to form a compound of formula (c). More specifically (a) and (b) are heated to a temperature between 80° C. to 130° C. in a solvent, such as toluene, with cesium carbonate, palladium acetate and 2-dicyclohexyl phosphino-2′,4′,6′-triisopropylbiphenyl (X-PHOS) under nitrogen for about 15-20 hours to form compound of formula (c).

Compounds of formula (c) are reacted with compounds formula (d) using conditions and reagents known to the skilled person (utilizing a mild base, such as triethylamine or pyridine) to afford compounds of formula (e). Compounds of formula (d) are prepared by the method of Tarasov, Evgeniy V.; Henckens, Anja; Ceulemans, Erik; Dehaen, Wim. A short total synthesis of cerpegin by intramolecular hetero Diels-Alder cycloaddition reaction of an acetylene tethered pyrimidine. Synlett (2000), (5), 625-626.

The t-butyldimethylsilyl group in compounds of formula (e) can be removed by one skilled in the art, utilizing conditions referenced in Greene's Protective Groups in Organic Synthesis, 4th Ed., P. G. M Wuts and T. W. Greene, Wiley-Interscience to afford compounds of formula (f). For t-butyldimethyl-silyl groups, deprotection can be accomplished by a range of conditions including acidic and fluoride-based conditions. Preferred conditions for t-butyldimethylsilyl are dilute aqueous hydrochloric acid in methanol at ambient temperature for 2-10 hours. Cyclization of compounds of formula (f) to produce compounds of formula (g) can be accomplished utilizing a wide range of basic conditions, including organic (e.g. triethylamine) and inorganic (e.g. potassium carbonate) as the bases, in an aprotic solvent at 20° C. to 120° C. to provide the cyclic lactam structure (g). Preferred conditions for this cyclization are triethylamine in acetonitrile at 40° C. to 120° C. for 4-16 hours. Conversion of compounds of formula (g) to the corresponding cyclic boronic acid ester may be carried out according to the procedure exemplified herein for Intermediate 8.

In Scheme B1, suitable aromatic substituents are those compatible with the reaction conditions.

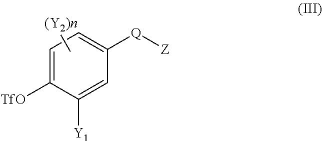

The compound of formula (III) can be prepared from the corresponding phenol compound by standard chemistry or from the corresponding methoxy compound after demethylation using BBr₃.

### Process B2

In an alternative sequence of Suzuki couplings compounds of formula (I) can be prepared by coupling of a compound of formula (II-i) and a compound of formula (III-i) to form a compound of formula (I) (wherein X represents trifluoromethanesulphonyl, iodo-, bromo- or chloro- and wherein E represents a boronic acid (—B(OH)₂), a boronate ester (—B(OR)₂ wherein R here is (1-4C)alkyl) or a cyclic boronic ester, such as pinacolato borane), as illustrated by the scheme below.

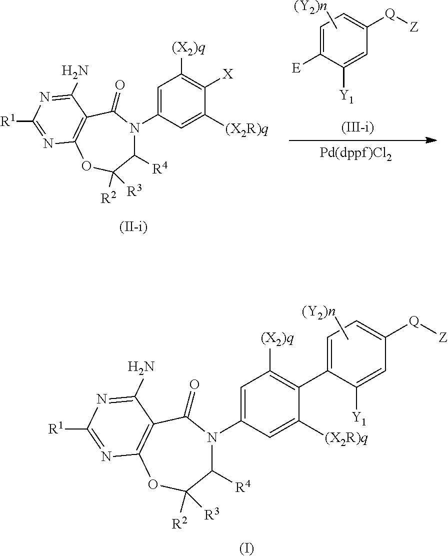

A compound of formula (II-i) can be prepared by processes to those described above in Scheme B1 above and a compound of formula (III-i) can be prepared by processes known to a person skilled in the art.

### Process B3

Alternatively a compound of formula (I) can be prepared as set out in Scheme B3 below. A compound of formula (IV) can be prepared by processes known in the art. The processes set out in Scheme B3 can be carried out by processes analogous to those described with reference to Scheme B1.

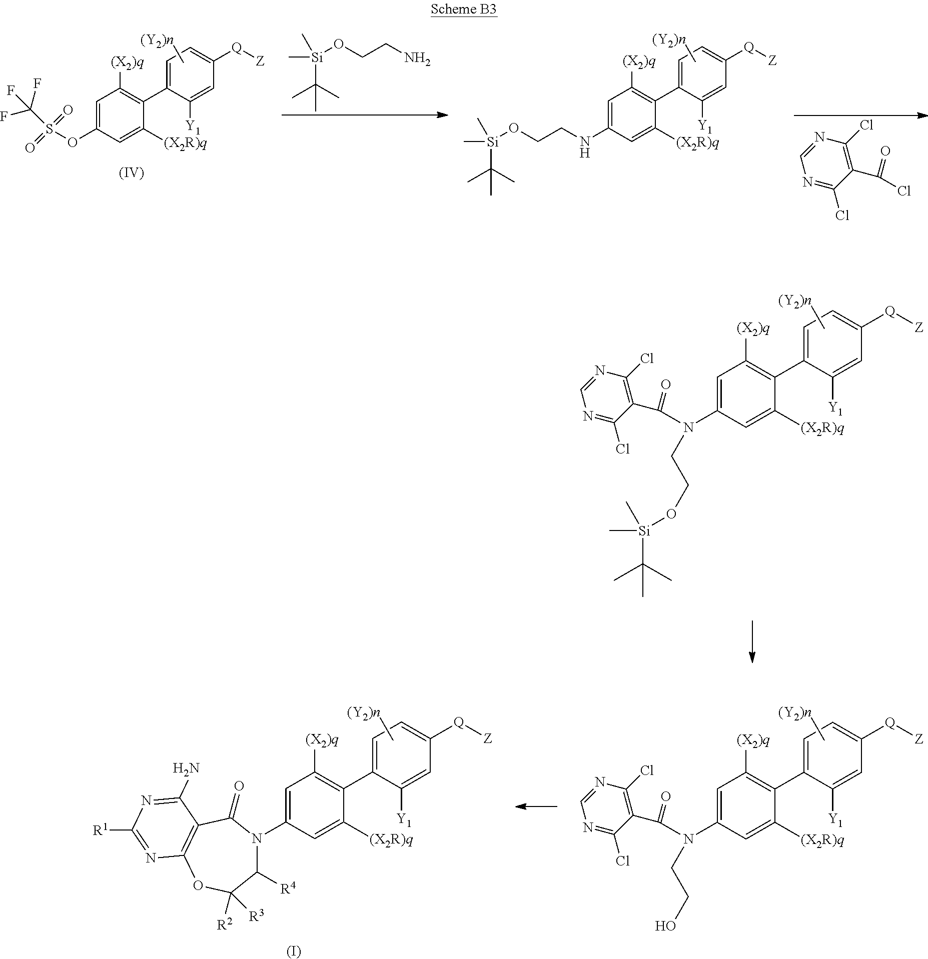

### Process B4

Alternatively a compound of formula (I) can be prepared as set out in Scheme B4 below. The processes set out in Scheme B4 can be carried out by processes analogous to those described with reference to Scheme B1 or by processes known in the art.

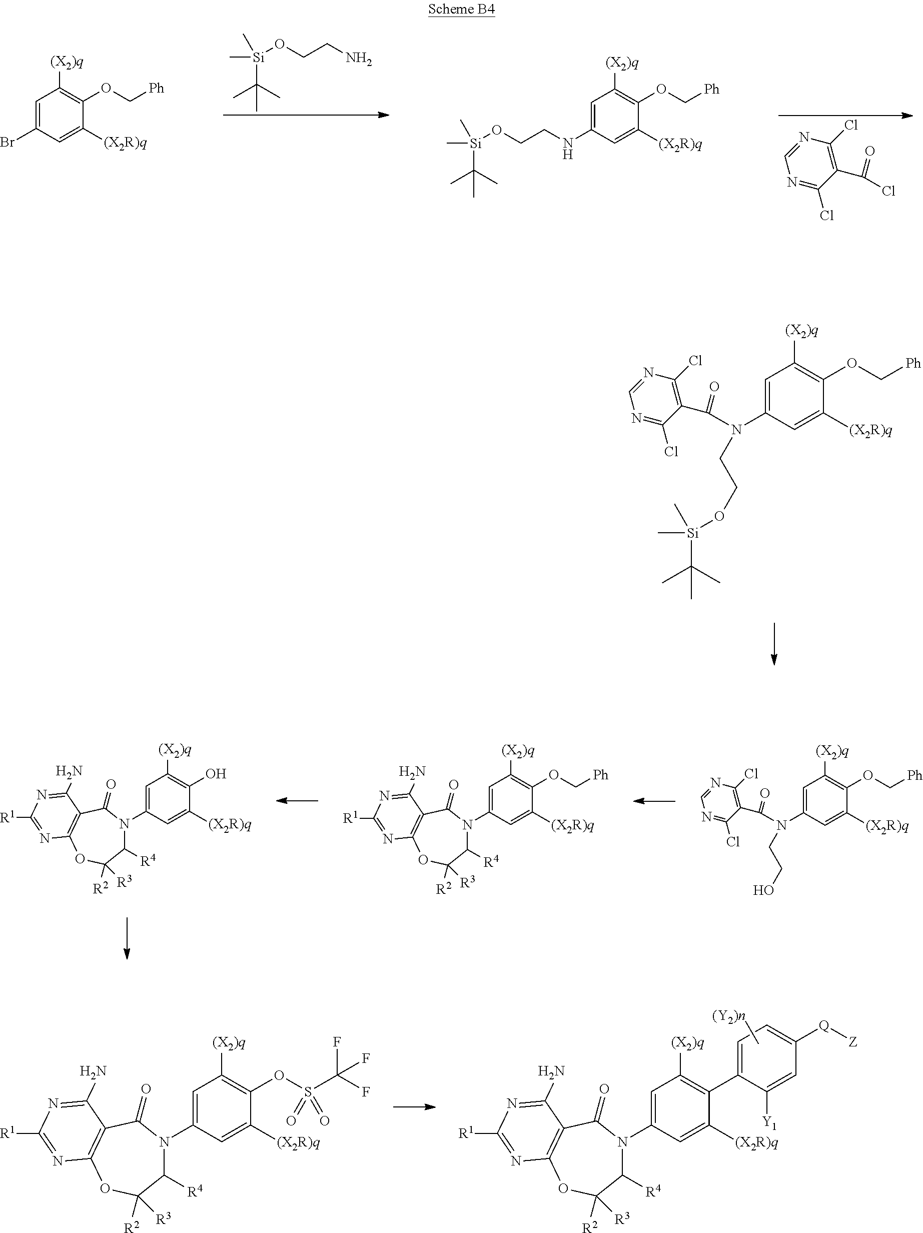

### Process C

The skilled man would be familiar with the synthesis of chiral compounds of the invention. Such chiral compounds also form an embodiment of the invention. Such chiral compounds may be made as follows:
(i) by chromatographic separation from a final mixture of compounds, for example using suitable hplc chiral stationary phases including Chiralpak OJ and AD columns;
(ii) by the use of chiral intermediates; or
(iii) by the use of chiral reduction methodologies.

If not commercially available, the necessary starting materials for the procedures such as those described above may be made by procedures which are selected from standard organic chemical techniques, techniques which are analogous to the synthesis of known, structurally similar compounds, techniques which are described or illustrated in the references given above, or techniques which are analogous to the above described procedure or the procedures described in the examples. The reader is further referred to Advanced Organic Chemistry, 5^{th} Edition, by Jerry March and Michael Smith, published by John Wiley & Sons 2001, for general guidance on reaction conditions and reagents.

It will be appreciated that some intermediates to compounds of the formula (I) are also novel and these are provided as separate independent aspects of the invention. In particular, certain compounds of formula (IV) may form a further independent aspect of the invention.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl or SEM may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The protecting groups may be removed at any convenient stage in the synthesis using conventional techniques well known in the chemical art, or they may be removed during a later reaction step or work-up.

The skilled organic chemist will be able to use and adapt the information contained and referenced within the above references, and accompanying Examples therein and also the examples herein, to obtain necessary starting materials, and products.

The removal of any protecting groups and the formation of a pharmaceutically-acceptable salt are within the skill of an ordinary organic chemist using standard techniques. Furthermore, details on these steps has been provided hereinbefore.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

According to a further aspect of the invention there is provided a pharmaceutical composition which comprises a compound of formula (I) as defined hereinbefore or a pharmaceutically-acceptable salt thereof, in association with a pharmaceutically-acceptable excipient or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl n-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

According to a further aspect of the present invention there is provided a compound of formula (I), or a pharmaceutically acceptable salt, or a pro-drug thereof as defined hereinbefore for use in a method of treatment of the human or animal body by therapy.

We have found that compounds of the present invention inhibit DGAT1 activity and are therefore of interest for their blood glucose-lowering and body weight-lowering effects.

A further feature of the present invention is a compound of formula (I), or a pharmaceutically-acceptable salt, or a pro-drug thereof for use as a medicament.

Conveniently this is a compound of formula (I), or a pharmaceutically-acceptable salt, or a pro-drug thereof, for (use as a medicament for) producing an inhibition of DGAT1 activity in a warm-blooded animal such as a human being.

Particularly this is a compound of formula (I), or a pharmaceutically-acceptable salt, or a pro-drug thereof, for (use as a medicament for) treating diabetes mellitus and/or obesity in a warm-blooded animal such as a human being.

Thus according to a further aspect of the invention there is provided the use of a compound of formula (I), or a pharmaceutically-acceptable salt, or a pro-drug thereof in the manufacture of a medicament for use in the production of an inhibition of DGAT1 activity in a warm-blooded animal such as a human being.

Thus according to a further aspect of the invention there is provided the use of a compound of formula (I), or a pharmaceutically-acceptable salt, or a pro-drug thereof in the manufacture of a medicament for use in the treatment of diabetes mellitus and/or obesity in a warm-blooded animal such as a human being.

According to a further aspect of the invention there is provided a pharmaceutical composition which comprises a compound of formula (I) as defined hereinbefore, or a pharmaceutically-acceptable salt, or a pro-drug thereof, in association with a pharmaceutically-acceptable excipient or carrier for use in producing an inhibition of DGAT1 activity in an warm-blooded animal, such as a human being.

According to a further aspect of the invention there is provided a pharmaceutical composition which comprises a compound of formula (I) as defined hereinbefore, or a pharmaceutically-acceptable salt, or a pro-drug thereof, in association with a pharmaceutically-acceptable excipient or carrier for use in the treatment of diabetes mellitus and/or obesity in an warm-blooded animal, such as a human being.

According to a further feature of the invention there is provided a method for producing an inhibition of DGAT1 activity in a warm-blooded animal, such as a human being, in need of such treatment which comprises administering to said animal an effective amount of a compound of formula (I), or a pharmaceutically-acceptable salt, or a pro-drug thereof as defined hereinbefore.

According to a further feature of the invention there is provided a method of treating diabetes mellitus and/or obesity in a warm-blooded animal, such as a human being, in need of such treatment which comprises administering to said animal an effective amount of a compound of formula (I), or a pharmaceutically-acceptable salt, or a pro-drug thereof as defined hereinbefore.

As stated above the size of the dose required for the therapeutic or prophylactic treatment of a particular disease state will necessarily be varied depending on the host treated, the route of administration and the severity of the illness being treated. Preferably a daily dose in the range of 0.1-50 mg/kg is employed. In another embodiment a daily dose is in the range of 0.01-50 mg/kg, particularly 0.01-10 mg/kg, 0.01-1 mg/kg or 0.01-0.1 mg/kg. However the daily dose will necessarily be varied depending upon the host treated, the particular route of administration, and the severity of the illness being treated.

Accordingly the optimum dosage may be determined by the practitioner who is treating any particular patient.

As stated above compounds defined in the present invention are of interest for their ability to inhibit the activity of DGAT1. A compound of the invention may therefore be useful for the prevention, delay or treatment of a range of disease states including diabetes mellitus, more specifically type 2 diabetes mellitus (T2DM) and complications arising there from (for example retinopathy, neuropathy and nephropathy), impaired glucose tolerance (IGT), conditions of impaired fasting glucose, metabolic acidosis, ketosis, dysmetabolic syndrome, arthritis, osteoporosis, obesity and obesity related disorders, (which include peripheral vascular disease, (including intermittent claudication), cardiac failure and certain cardiac myopathies, myocardial ischaemia, cerebral ischaemia and reperfusion, hyperlipidaemias, atherosclerosis, infertility and polycystic ovary syndrome); the compounds of the invention may also be useful for muscle weakness, diseases of the skin such as acne, various immunomodulatory diseases (such as psoriasis), HIV infection, inflammatory bowel syndrome and inflammatory bowel disease such as Crohn's disease and ulcerative colitis.

In particular, the compounds of the present invention are of interest for the prevention, delay or treatment of diabetes mellitus and/or obesity and/or obesity related disorders. In one aspect, the compounds of the invention are used for prevention, delay or treatment of diabetes mellitus. In another aspect, the compounds of the invention are used for prevention, delay or treatment of obesity. In a further aspect, the compounds of the invention are used for prevention, delay or treatment of obesity related disorders.

The inhibition of DGAT1 activity described herein may be applied as a sole therapy or in combination with one or more other substances and/or treatments for the indication being treated. Such conjoint treatment may be achieved by way of the simultaneous, sequential or separate administration of the individual components of the treatment. Simultaneous treatment may be in a single tablet or in separate tablets (or other dosage forms). For example such conjoint treatment may be beneficial in the treatment of metabolic syndrome [defined as abdominal obesity (as measured by waist circumference against ethnic and gender specific cut-points) plus any two of the following: hypertriglyceridemia (>150 mg/dl; 1.7 mmol/l); low HDLc (<40 mg/dl or <1.03 mmol/l for men and <50 mg/dl or 1.29 mmol/l for women) or on treatment for low HDL (high density lipoprotein); hypertension (SBP≧130 mmHg DBP≧85 mmHg) or on treatment for hypertension; and hyperglycemia (fasting plasma glucose≧100 mg/dl or 5.6 mmol/l or impaired glucose tolerance or pre-existing diabetes mellitus)—International Diabetes Federation & input from IAS/NCEP].

Such conjoint treatments may include the following main categories:

1) Anti-obesity therapies such as those that cause weight loss by effects on food intake, nutrient absorption or energy expenditure, such as orlistat, sibutramine and the like.
2) Insulin secretagogues including sulphonylureas (for example glibenclamide, glipizide), prandial glucose regulators (for example repaglinide, nateglinide);
3) Agents that improve incretin action (for example dipeptidyl peptidase IV inhibitors such as saxagliptin, and GLP-1 agonists);
4) Insulin sensitising agents including PPARgamma agonists (for example pioglitazone and rosiglitazone), and agents with combined PPARalpha and gamma activity;
5) Agents that modulate hepatic glucose balance (for example metformin, fructose 1,6 bisphosphatase inhibitors, glycogen phopsphorylase inhibitors, glycogen synthase kinase inhibitors, glucokinase activators);
6) Agents designed to reduce the absorption of glucose from the intestine (for example acarbose);
7) Agents that prevent the reabsorption of glucose by the kidney (SGLT inhibitors, such as dapagliflozin);
8) Agents designed to treat the complications of prolonged hyperglycaemia (for example aldose reductase inhibitors);
9) Anti-dyslipidaemia agents such as, HMG-CoA reductase inhibitors (eg statins); PPAR α-agonists (fibrates, eg gemfibrozil); bile acid sequestrants (cholestyramine); cholesterol absorption inhibitors (plant stanols, synthetic inhibitors); bile acid absorption inhibitors (IBATi) and nicotinic acid and analogues (niacin and slow release formulations);
10) Antihypertensive agents such as β-blockers (eg atenolol, inderal (propranolol)); ACE inhibitors (eg lisinopril); Calcium antagonists (eg. nifedipine); Angiotensin receptor antagonists (eg candesartan), α-antagonists and diuretic agents (eg. furosemide, benzthiazide);
11) Haemostasis modulators such as, antithrombotics, activators of fibrinolysis (streptokinase, alteplas) and antiplatelet agents; thrombin antagonists; factor Xa inhibitors; factor VIIa inhibitors); antiplatelet agents (eg. aspirin, clopidogrel); anticoagulants (heparin and Low molecular weight analogues, hirudin) and warfarin;
12) Agents which antagonise the actions of glucagon; and
13) Anti-inflammatory agents, such as non-steroidal anti-inflammatory drugs (eg. aspirin, ibuprofen) and steroidal anti-inflammatory agents (eg. cortisone).

It will be understood that insulin may also be required as a conjoint treatment.

In addition to their use in therapeutic medicine, compounds of formula (I) and their pharmaceutically-acceptable salts are also useful as pharmacological tools in the development and standardisation of in vitro and in vivo test systems for the evaluation of the effects of inhibitors of DGAT1 activity in laboratory animals such as cats, dogs, rabbits, monkeys, rats and mice, as part of the search for new therapeutic agents.

In the above other pharmaceutical composition, process, method, use and medicament manufacture features, the alternative, particular and preferred embodiments of the compounds of the invention described herein also apply. The alternative, particular and preferred embodiments of the invention described herein also apply to a compound of formula (I), or a pharmaceutically-acceptable salt, or a pro-drug thereof.

As indicated above, the compounds, and their corresponding pharmaceutically-acceptable salts, are useful in inhibiting DGAT1. The ability of the compounds of formula (I), and their corresponding pharmaceutically-acceptable (acid addition) salts, to inhibit DGAT1 may be demonstrated employing the following enzyme assay:

### Human Enzyme Assay

See, for example, International Application WO 2005/044250.

The in vitro assay to identify DGAT1 inhibitors uses human DGAT1 expressed in insect cell membranes as the enzyme source (Proc. Natl. Acad. Sci. 1998, 95, 13018-13023). Briefly, sf9 cells were infected with recombinant baculovirus containing human DGAT1 coding sequences and harvested after 48 h. Cells were lysed by sonication and membranes isolated by centrifuging at 28000 rpm for 1 h at 4° C. on a 41% sucrose gradient. The membrane fraction at the interphase was collected, washed, and stored in liquid nitrogen.

DGAT1 activity was assayed by a modification of the method described by Coleman (Methods in Enzymology 1992, 209, 98-102). Compound at 0.0000256 μM (or 0.003 μM)-33 μM (final conc.) (typically 10 μM) was incubated with 4 μg/mL (final conc) membrane protein, 5 mM MgCl₂, and 100 μM 1,2 dioleoyl-sn-glycerol (dissolved in acetone with a final assay conc. of acetone of 10%) in a total assay volume of 200 μl in a 96 well plate. The reaction was started by adding ¹⁴C oleoyl coenzyme A (30 μM final concentration) and incubated at room temperature for 30 minutes. The reaction was stopped by adding 200 μl 2-propanol:heptane 7:1. Radioactive triolein product was separated into the organic phase by adding 300 μl heptane and 100 μl 0.1 M carbonate buffer pH 9.5. DGAT1 activity was quantified by counting aliquots of the upper heptane layer by liquid scintillography.

Using this assay the compounds generally show activity with an IC₅₀ around or below 10 μM, preferably below 10 μM (i.e. IC₅₀<10 μM), preferably <1 μM, more preferably <0.1 μM, particularly, <0.05 μM, and more particularly <0.01 μM. Stated figures are usually a mean of a number of measurements (usually 2 measurements) according to standard practice.

Examples 1 to 4 showed, respectively, an IC₅₀=0.017 μM; 0.034 μM; 0.033 μM, 0.035 μM.

Examples 5 to 24 showed the following IC₅₀ values (rounded to 2 decimal places)

| Example number | IC₅₀ (μM) |
|---|---|
| 5 | 0.01 |
| 6 | 0.40 |
| 7 | 0.08 |
| 8 | 0.07 |
| 9 | 0.02 |
| 10 | 0.10 |
| 11 | 0.05 |
| 12 | 0.26 |
| 13 | 0.04 |
| 14 | 0.10 |
| 15 | 0.24 |
| 16 | 0.01 |
| 17 | 0.02 |
| 18 | 0.04 |
| 19 | 0.05 |
| 20 | 0.02 |
| 21 | 0.03 |
| 22 | 0.05 |
| 23 | 0.04 |
| 24 | 0.07 |

Examples 25 to 59 showed the following IC₅₀ values (rounded to 2 decimal places)

| Example number | IC₅₀ (μM) |
|---|---|
| 25 | 0.32 |
| 26 | 0.11 |
| 27 | 0.04 |
| 28 | 0.13 |
| 29 | 0.06 |
| 30 | 0.12 |
| 31 | 0.02 |
| 32 | 0.05 |
| 33 | 0.45 |
| 34 | 0.04 |
| 36 | 0.03 |
| 37 | 0.03 |
| 38 | 0.07 |
| 39 | 0.04 |
| 40 | 0.06 |
| 41 | 0.05 |
| 42 | 0.09 |
| 43 | 0.10 |
| 44 | 0.02 |
| 45 | 0.06 |
| 46 | 0.02 |
| 47 | 0.05 |
| 48 | 0.06 |
| 49 | 0.02 |
| 50 | 0.04 |
| 51 | 0.02 |
| 52 | 0.07 |
| 53 | 0.02 |
| 54 | 0.02 |
| 55 | 0.01 |
| 56 | 0.01 |
| 57 | 0.02 |
| 58 | 0.01 |
| 59 | 0.02 |

The ability of the compounds of formula (I), and their corresponding pharmaceutically-acceptable (acid) salts, to inhibit DGAT1 may further be demonstrated employing the following whole cell assay.

### Measurement of Triglyceride Synthesis in HuTu 80 Cells

HuTu80 cells were cultured to confluency in 6 well plates in minimum essential media containing foetal calf serum. For the experiment, the medium was changed to serum-free medium and the cells pre-incubated with compound solubilised in DMSO (final concentration 0.1%) for 30 minutes. De novo lipogenesis was measured by the addition of 0.12 mM sodium oleate plus 1 μCi/mL ¹⁴C-sodium oleate complexed to 0.03 mM BSA to each well for a further 2 h. The cells were washed in phosphate buffered saline and solubilised in 1% sodium dodecyl sulfate. An aliquot was removed for protein determination using a protein estimation kit (Perbio) based on the method of Lowry (J. Biol. Chem., 1951, 193, 265-275). The lipids were extracted into the organic phase using a heptane:propan-2-ol:water (80:20:2) mixture followed by aliquots of water and heptane according to the method of Coleman (Methods in Enzymology, 1992, 209, 98-104). The organic phase was collected and the solvent evaporated under a stream of nitrogen. The extracts solubilised in iso-hexane:acetic acid (99:1) and lipids separated via normal phase high performance liquid chromatography (HPLC) using a Lichrospher diol-5, 4×250 mm column and a gradient solvent system of iso-hexane:acetic acid (99:1) and iso-hexane:propan-2-ol:acetic acid (85:15:1), flow rate of 1 mL/minute according to the method of Silversand and Haux (1997). Incorporation of radiolabel into the triglyceride fraction was analysed using a Radiomatic Flo-one Detector (Packard) connected to the HPLC machine.

### EXAMPLES

The following examples are for illustration purposes and are not intended to limit the scope of this application. Each exemplified compound represents a particular and independent aspect of the invention. In the following non-limiting Examples, unless otherwise stated:

(i) evaporations were carried out by rotary evaporation under reduced pressure and work-up procedures were carried out after removal of residual solids such as drying agents by filtration;
(ii) operations were carried out at room temperature, that is in the range 18-25° C. and generally under an atmosphere of an inert gas such as argon or nitrogen;
(iii) yields are given for illustration only and are not necessarily the maximum attainable;
(iv) the structures of the end-products of the formula (I) were confirmed by nuclear (generally proton) magnetic resonance (NMR) and mass spectral techniques; proton magnetic resonance chemical shift values were measured on the delta scale and peak multiplicities are shown as follows: s, singlet; d, doublet; t, triplet; m, multiplet; br, broad; q, quartet, quin, quintet;
(v) intermediates were not generally fully characterised and purity was assessed by thin layer chromatography (TLC), high-performance liquid chromatography (HPLC), infra-red (IR) or NMR analysis;
(vi) flash chromatography was carried out on silica unless otherwise stated with flash chromatography purifications run on Biotage SP1 or SP4 instruments using Biotage Silica columns;
(vii) mass spectra were recorded on a Finnigan LCQ Duo ion trap mass spectrometer equipped with an electrospray interface (LC-MS) or LC-MS system consisting of a Waters ZQ using a LC-Agilent 1100 LC system;
(viii) ¹H NMR measurements were performed on a Varian Mercury VXR 300 and 400 spectrometer, operating at a 1H frequency of 300 and 400 and Varian UNITY plus 400, 500 and 600 spectrometers, operating at 1H frequencies of 400, 500 and 600 respectively. Chemical shifts are given in ppm with the solvent as internal standard. Protons on heteroatoms such as NH and OH protons are only reported when detected in NMR and can therefore be missing.
(ix) Unless stated otherwise, HPLC separations were performed on a Waters YMC-ODS AQS-3 120 Angstrom 3×500 mm or on a Waters Delta Prep Systems using Kromasil C8, 10 μm columns. Unless stated otherwise, acidic HPLC was carried out using gradients of mobilephase A: 100% ACN and mobilephase B: 5% ACN+95% H₂O+0.2% FA; neutral HPLC was carried out using gradients of mobilephase A: 100% ACN and mobilephase B: 5% ACN+95% 0.1 M NH₄OAc.
(x) Reactions performed in a microwave oven were run in a Biotage Initiator Instrument.
(xi) Chemical nomenclature software packages, such as Struc=Name/CambridgeSoft ELN, may have been used in the naming of compounds.
List of Abbreviations that May be Used Herein:

| | |
|---|---|
| ACN | Acetonitrile |
| aq | Aqueous |
| Boc | tert-butyloxycarbonyl |
| Brine | Saturated solution of sodium chloride in water |
| BSA | Bovine Serum Albumine |
| Cbz | Benzylozycarbonyl |
| DCE | 1,2-dichloroethane |
| DCM | Dichloromethane |
| DEE | Diethylether |
| DIPEA | N,N-Diisopropylethylamine |
| DMAP | Dimethylaminopyridine |
| DMF | N,N-dimethylformamide |
| DMSO | Dimethylsulphoxide |
| Dppf | 1,1′-bis(Diphenylphosphino)ferrocene |
| EDCI | 1-Ethyl-3-(3-dimethylaminopropyl)carbodiimide |
| | hydrochloride |
| EDTA | Ethylenediaminetetraacetic acid |
| EtOAc | Ethyl acetate |
| EtOH | Ethanol |
| FA | Formic acid |
| HOAc | Acetic acid |
| HPLC | High-performance liquid chromatography |
| HWE | Horner-Wadsworth-Emmons |
| Hz | Hertz |
| IPA | Isopropylalcohol |
| iPr | isopropyl |
| LC | Liquid chromatography |
| m-CPBA | meta-chloroperoxybenzoicacid |
| MeOH | Methanol |
| Methyl THF | 2-methyltetrahydrofuran |
| MHz | Megahertz |
| mL | Millilitre |
| MS | Mass spectra |
| NMM | N-methylmorpholine |
| NMP | N-methylpiperazine |
| NMR | Nuclear magnetic resonance |
| OAc | acetate |
| Ph | Phenyl |
| PyBOP | Benzotriazol-1-yl-oxytri-pyrrolidinophosphonium |
| | hexafluorophosphate |
| PyBROP | Bromo-tris-pyrrolidino-phosphonium |
| | Hexafluorophosphate |
| Ps-Py—SO₃ | Polymer supported pryridine-SO₃ complex |
| RT | Room temperature |
| sat | saturated |
| TEA | Triethylamine |
| Tf | trifluoromethylsulfonyl |
| TFA | Trifluoroacetic acid |
| THF | Tetrahydrofuran |
| TLC | Thin layer chromatography |
| Ts | p-toluenesulfonyl |

### Example 1

### (R)-4-amino-6-(2′-chloro-4′-(2-(3-hydroxypyrrolidin-1-yl)-2-oxoethyl)biphenyl-4-yl)-7,8-dihydropyrimido[5,4-f][1,4]oxazepin-5(6H)-one

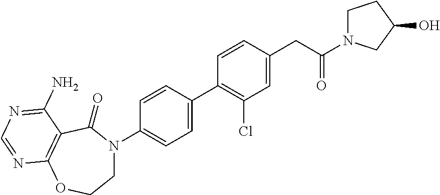

O-(7-Azabenzotriazol-1-yl)-N,N,N′,N′-tetramethyluronium hexafluorophosphate (175 mg, 0.46 mmol) was added in one portion to 2-(4′-(4-amino-5-oxo-7,8-dihydropyrimido[5,4-f][1,4]oxazepin-6(5H)-yl)-2-chlorobiphenyl-4-yl)acetic acid (Intermediate 1; 150 mg, 0.35 mmol), (R)-pyrrolidin-3-ol hydrochloride (48.0 mg, 0.39 mmol) and N,N-diisopropylethylamine (0.151 mL, 0.88 mmol) in DMF (2 mL) under nitrogen. The resulting solution was stirred at ambient temperature for 2 hours. The reaction mixture was concentrated and diluted with a 50:50 mix of THF and EtOAc (100 mL), and washed sequentially with water (2×50 mL) and saturated brine (2×50 mL). The organic layer was dried over Na₂SO₄, filtered and evaporated to afford crude product. The crude product was purified by flash silica chromatography, elution gradient 0 to 25% MeOH in DCM. Pure fractions were evaporated to dryness to afford (R)-4-amino-6-(2′-chloro-4′-(2-(3-hydroxypyrrolidin-1-yl)-2-oxoethyl)biphenyl-4-yl)-7,8-dihydropyrimido[5,4-f][1,4]oxazepin-5(6H)-one (60.8 mg, 34.9%) as a white crystalline solid.

¹H NMR (400 MHz, DMSO) δ 1.72-1.99 (2H, m), 3.16-3.28 (1H, m), 3.31-3.45 (2H, m), 3.58-3.71 (3H, m), 4.05 (2H, t), 4.29 (1H, d), 4.63 (2H, t), 4.90-5.02 (1H, m), 7.29 (1H, d), 7.37 (1H, d), 7.45 (1H, d), 7.46-7.51 (4H, m), 7.62 (2H, s), 8.18 (1H, s) m/z (ES+) (M+H)+=494, 496

### Examples 2-3

Were prepared by analogous method to example 1, by coupling of intermediate 1 with the appropriate amine, see table.

| | | ¹H NMR (400 MHz, | Mass |
|---|---|---|---|
| | Structure | DMSO) | Spec |
| Example 2: 4- amino-6-(2′-chloro- 4′-(2-oxo-2-(3- oxopiperazin-1- yl)ethyl)biphenyl-4- yl)-7,8- dihydropyrimido[5,4- f][1,4]oxazepin- 5(6H)-one | 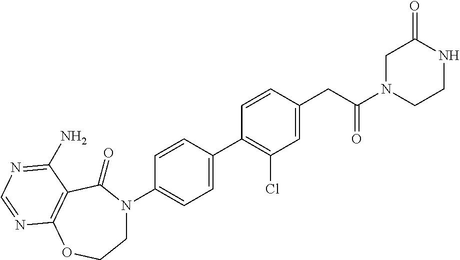 | δ 3.22 (2H, d), 3.63 (1H, t), 3.72 (1H, t), 3.82 (2H, d), 3.96 (1H, s), 4.05 (2H, t), 4.13 (1H, s), 4.63 (2H, t), 7.26-7.29 (1H, m), 7.37 (1H, d), 7.43-7.48 (1H, m), 7.49 (4H, s), 7.62 (2H, s), 8.03 (1H, t), 8.18 (1H, s) | m/z (ES+) (M + H)+ = 507, 509 |
| Example 3: 4- amino-6-(2′-chloro- 4′-(2-(3- hydroxyazetidin-1- yl)-2- oxoethyl)biphenyl-4- yl)-7,8- dihydropyrimido[5,4- f][1,4]oxazepin- 5(6H)-one | 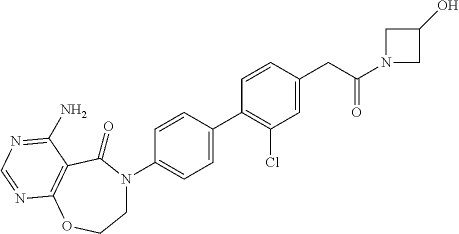 | δ 3.49 (2H, s), 3.59 (1H, dd), 3.94 (1H, dd), 4.05 (3H, t), 4.37-4.43 (1H, m), 4.46 (1H, dd), 4.61- 4.65 (2H, m), 5.71 (1H, d), 7.29 (1H, dd), 7.37 (1H, d), 7.45 (1H, d), 7.45-7.52 (4H, m), 7.62 (2H, s), 8.18 (1H, s). | m/z (ES+) (M + H)+ = 480, 482 |

### Example 4

### 4′-(4-Amino-5-oxo-7,8-dihydropyrimido[5,4-f][1,4]oxazepin-6(5H)-yl)-2-chlorobiphenyl-4-sulfonamide

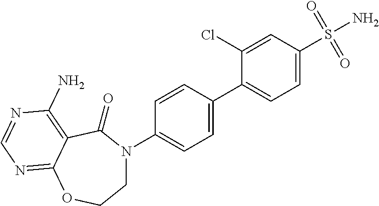

4-Amino-6-(4-(4,4,5,5-tetramethyl-1,3,2-dioxaborolan-2-yl)phenyl)-7,8-dihydropyrimido[5,4-f][1,4]oxazepin-5(6H)-one (Intermediate 8; 314 mg, 0.82 mmol), 4-bromo-3-chlorobenzenesulfonamide (222 mg, 0.82 mmol) and tripotassium phosphate (209 mg, 0.99 mmol) were suspended in a mixture of DME (10 mL), methanol (5.0 mL) and water (0.250 mL) and sealed into a microwave tube. The mixture was degassed under vacuum, treated with (1,1′-bis(diphenylphosphino)ferrocene)-dichloropalladium(II) (DCM adduct) (33.8 mg, 0.04 mmol) and re-sealed. The reaction was heated to 80° C. for 35 minutes in the microwave reactor then cooled to RT. The reaction mixture was evaporated to dryness and redissolved in DCM (50 mL), and washed sequentially with 1M NaOH (20 mL), water (10 mL) and saturated brine (10 mL). The organic layer was dried over Na₂SO₄, filtered and evaporated to afford crude product. The crude product was purified by flash silica chromatography, elution gradient 0 to 20% MeOH in DCM. Fractions were evaporated to dryness to afford a cleaner product which was ˜60% pure. The crude product was further purified by preparative HPLC (Waters XBridge Prep C18 OBD column, 5μ, silica, 50 mm diameter, 150 mm length), using decreasingly polar mixtures of water (containing 0.1% formic acid) and MeCN as eluents. Fractions containing the desired compound were evaporated to dryness to afford 4′-(4-amino-5-oxo-7,8-dihydropyrimido[5,4-f][1,4]oxazepin-6(5H)-yl)-2-chlorobiphenyl-4-sulfonamide (23.70 mg, 6.47%) as a white solid. ¹H NMR (400 MHz, DMSO,) δ 4.02-4.10 (2H, m), 4.59-4.67 (2H, m), 7.51-7.58 (6H, m), 7.63 (2H, s), 7.66 (1H, d), 7.84 (1H, dd), 7.97 (1H, d), 8.18 (1H, s). m/z (ES+) (M+H)+=446, 448

### Example 5

### 4-Amino-6-(2′,4′-dichlorobiphenyl-4-yl)-7,8-dihydropyrimido[5,4-f][1,4]oxazepin-5(6H)-one

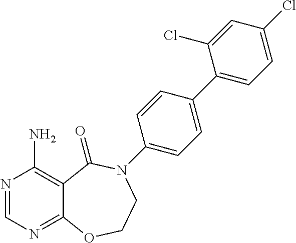

4-Amino-6-(4-bromophenyl)-7,8-dihydropyrimido[5,4-f][1,4]oxazepin-5(6H)-one (Intermediate 3; 300 mg, 0.90 mmol), 2,4-dichlorophenylboronic acid (171 mg, 0.90 mmol) and tripotassium phosphate (228 mg, 1.07 mmol) were suspended in DME (8.0 mL), methanol (4.0 mL) and water (2.0 mL) and sealed into a microwave tube. The mixture was degassed under vacuum and the atmosphere replaced with nitrogen. (1,1′-Bis(diphenylphosphino)ferrocene)-dichloropalladium(II) (DCM adduct) (36.8 mg, 0.04 mmol) was added and the reaction was heated to 95° C. for 35 minutes in the microwave reactor and cooled to RT. The reaction mixture was evaporated to dryness and redissolved in EtOAc (50 mL), and washed with water (20 mL). The organic layer was dried over Na₂SO₄, filtered and evaporated to afford crude product. The crude product was purified by flash silica chromatography, elution gradient 30 to 100% EtOAc in isohexanes. Pure fractions were evaporated to dryness to afford 4-amino-6-(2′,4′-dichlorobiphenyl-4-yl)-7,8-dihydropyrimido[5,4-f][1,4]oxazepin-5(6H)-one (241 mg, 67.1%) as a beige solid.

¹H NMR (400 MHz, DMSO) δ 4.10-4.14 (2H, m), 4.68-4.72 (2H, m), 7.54 (1H, d), 7.57 (4H, s), 7.58-7.62 (1H, m), 7.69 (2H, s), 7.82 (1H, d), 8.25 (1H, s). m/z (ES+) (M+H)+=401, 403

### Examples 6-17

Prepared following the method of example 5, coupling Intermediate 3 with the appropriate boronic acid.

| | | ¹H NMR (400 | Mass |
|---|---|---|---|
| Example | Structure | MHz) | Spec |
| 6 4-amino-6-(3′-chloro- 2′-methylbiphenyl-4- yl)-7,8- dihydropyrimido[5,4- f][1,4]oxazepin-5(6H)- one | 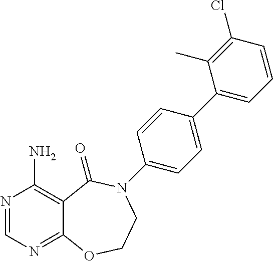 | (DMSO) δ 2.27 (3H, s), 4.02-4.09 (2H, m), 4.61- 4.66 (2H, m), 7.21 (1H, d), 7.29 (1H, t), 7.40 (2H, d), 7.44-7.49 (3H, m), 7.62 (2H, s), 8.18 (1H, s). | m/z (ES+) (M + H)+ = 381, 383 |
| 7 4-amino-6-(4′-chloro- 2′-methylbiphenyl-4- yl)-7,8- dihydropyrimido[5,4- f][1,4]oxazepin-5(6H)- one | 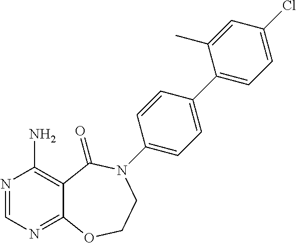 | (DMSO) δ 2.26 (3H, s), 4.02-4.06 (2H, m), 4.61- 4.65 (2H, m), 7.24 (1H, d), 7.30- 7.34 (1H, m), 7.38- 7.42 (3H, m), 7.46 (2H, d), 7.62 (2H, s), 8.18 (1H, s). | m/z (ES+) (M + H)+ = 381, 383 |
| 8 4-amino-6-(5′-chloro- 2′-methylbiphenyl-4- yl)-7,8- dihydropyrimido[5,4- f][1,4]oxazepin-5(6H)- one | 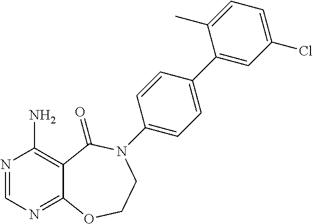 | (DMSO) δ 2.23 (3H, s), 4.02-4.07 (2H, m), 4.61- 4.65 (2H, m), 7.26 (1H, s), 7.34 (2H, d), 7.40-7.44 (2H, m), 7.45- 7.50 (2H, m), 7.62 (2H, s), 8.18 (1H, s). | m/z (ES+) (M + H)+ = 381, 383 |
| 9 4-amino-6-(2′,5′- dichlorobiphenyl-4- yl)-7,8- dihydropyrimido[5,4- f][1,4]oxazepin-5(6H)- one | 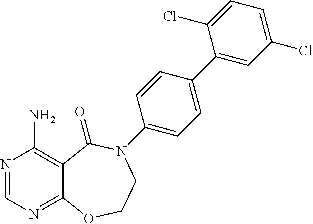 | (DMSO) δ 4.03- 4.07 (2H, m), 4.61- 4.65 (2H, m), 7.47-7.54 (6H, m), 7.60-7.64 (3H, m), 8.18 (1H, s). | m/z (ES+) (M + H)+ = 401, 403, 405 |
| 10 4-amino-6-(2′,3′- dichlorobiphenyl-4- yl)-7,8- dihydropyrimido[5,4- f][1,4]oxazepin-5(6H)- one | 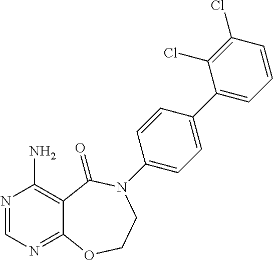 | (DMSO) δ 4.10- 4.14 (2H, m), 4.68- 4.72 (2H, m), 7.54 (1H, d), 7.57 (4H, s), 7.58-7.62 (1H, m), 7.69 (2H, s), 7.82 (1H, d), 8.25 (1H, s). | m/z (ES+) (M + H)+ = 401, 403, 405 |
| 11 4-amino-6-(2′- methylbiphenyl-4-yl)- 7,8- dihydropyrimido[5,4- f][1,4]oxazepin-5(6H)- one | 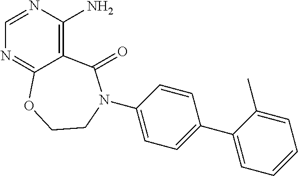 | (CDCl₃) δ 2.31 (3H, s), 4.08 (2H, dd), 4.72-4.76 (2H, m), 5.63 (1H, s), 7.21-7.30 (4H, m), 7.32-7.35 (2H, m), 7.39- 7.45 (2H, m), 8.10- 8.27 (1H, m), 8.30 (1H, s). | m/z (ES+) (M + H)+ = 347 |
| 12 4-amino-6-(biphenyl- 4-yl)-7,8- dihydropyrimido[5,4- f][1,4]oxazepin-5(6H)- one | 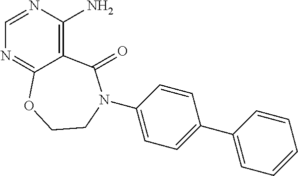 | (CDCl₃) δ 4.05- 4.10 (2H, m), 4.68- 4.78 (2H, m), 5.62 (1H, s), 7.34- 7.40 (3H, m), 7.43- 7.48 (2H, m), 7.54-7.61 (2H, m), 7.62-7.70 (2H, m), 8.10- 8.27 (1H, m), 8.30 (1H, s). | m/z (ES+) (M + H)+ = 333 |
| 13 4-amino-6-(2′- fluorobiphenyl-4-yl)- 7,8- dihydropyrimido[5,4- f][1,4]oxazepin-5(6H)- one | 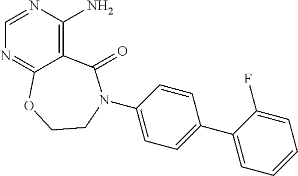 | (CDCl₃) δ 4.08 (2H, dd), 4.70- 4.75 (2H, m), 5.64 (1H, s), 7.13-7.24 (2H, m), 7.31- 7.40 (3H, m), 7.44 (1H, td), 7.61- 7.68 (2H, m), 8.09- 8.26 (1H, m), 8.30 (1H, s). | m/z (ES+) (M + H)+ = 351 |
| 14 4′-(4-amino-5-oxo-7,8- dihydropyrimido[5,4- f][1,4]oxazepin-6(5H)- yl)-5-chlorobiphenyl- 2-carbonitrile | 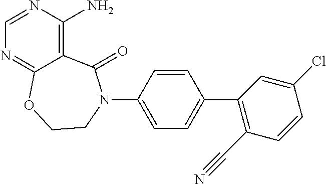 | (CDCl₃) δ 4.02- 4.15 (2H, m), 4.65- 4.79 (2H, m), 5.83 (1H, s), 7.40- 7.50 (3H, m), 7.53 (1H, t), 7.62-7.69 (2H, m), 7.72 (1H, d), 8.17 (1H, s), 8.29 (1H, d). | m/z (ES+) (M + H)+ = 392 |
| 15 4′-(4-amino-5-oxo-7,8- dihydropyrimido[5,4- f][1,4]oxazepin-6(5H)- yl)-3-chlorobiphenyl- 2-carbonitrile | 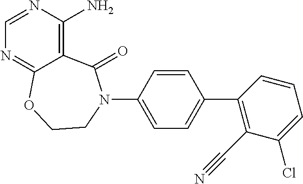 | (DMSO) δ 3.93- 4.05 (2H, m), 4.51- 4.62 (2H, m), 7.52 (5H, ddt), 7.57-7.65 (2H, m), 7.66-7.79 (2H, m), 8.10 (1H, s). | m/z (ES+) (M + H)+ = 392 |
| 16 4-amino-6-(2′,6′- difluorobiphenyl-4- yl)-7,8- dihydropyrimido[5,4- f][1,4]oxazepin-5(6H)- one | 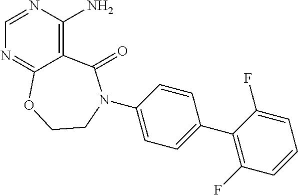 | (CDCl₃) δ 4.04- 4.11 (2H, m), 4.70- 4.76 (2H, m), 5.63 (1H, s), 6.97- 7.04 (2H, m), 7.31 (1H, ddd), 7.36- 7.42 (2H, m), 7.58 (2H, d), 8.08- 8.26 (1H, m), 8.30 (1H, s). | m/z (ES+) (M + H)+ = 369 |
| 17 4-amino-6-(2′- chlorobiphenyl-4-yl)- 7,8- dihydropyrimido[5,4- f][1,4]oxazepin-5(6H)- one | 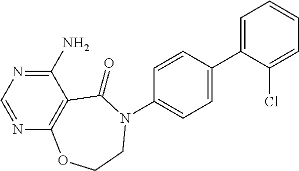 | (DMSO) δ 4.01- 4.09 (2H, m), 4.59- 4.68 (2H, m), 7.37-7.53 (7H, m), 7.54-7.68 (3H, m), 8.18 (1H, s). | m/z (ES+) (M + H)+ = 367 |

### Example 18

### 4′-(4-Amino-5-oxo-7,8-dihydropyrimido[5,4-f][1,4]oxazepin-6(5H)-yl)-2-chlorobiphenyl-4-sulfonamide

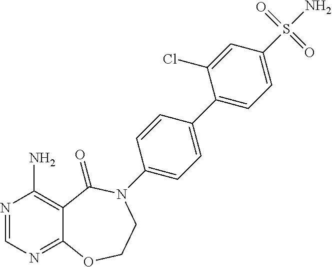

4-Amino-6-(4-(4,4,5,5-tetramethyl-1,3,2-dioxaborolan-2-yl)phenyl)-7,8-dihydropyrimido[5,4-f][1,4]oxazepin-5(6H)-one (Intermediate 8; 314 mg, 0.82 mmol), 4-bromo-3-chlorobenzenesulfonamide (222 mg, 0.82 mmol) and tripotassium phosphate (209 mg, 0.99 mmol) were suspended in a mixture of DME (10 mL), methanol (5.0 mL) and water (0.25 mL) and sealed into a microwave tube. The mixture was degassed under vacuum, treated with (1,1′-bis(diphenylphosphino)ferrocene)-dichloropalladium(II) (DCM adduct) (33.8 mg, 0.04 mmol) and re-sealed. The reaction was heated to 80° C. for 35 minutes in the microwave reactor and cooled to RT. The reaction mixture was evaporated to dryness and redissolved in DCM (50 mL), and washed sequentially with 1M NaOH (20 mL), water (10 mL) and saturated brine (10 mL). The organic layer was dried over Na₂SO₄, filtered and evaporated to afford crude product. The crude product was purified by flash silica chromatography, elution gradient 0 to 20% MeOH in DCM. Fractions were evaporated to dryness to afford a cleaner product which was ˜60% pure. The crude product was combined with that from a second preparation and purified by preparative HPLC (Waters XBridge Prep C18 OBD column, 5μ silica, 50 mm diameter, 150 mm length), using decreasingly polar mixtures of water (containing 0.1% formic acid) and MeCN as eluents. Fractions containing the desired compound were evaporated to dryness to afford 4′-(4-amino-5-oxo-7,8-dihydropyrimido[5,4-f][1,4]oxazepin-6(5H)-yl)-2-chlorobiphenyl-4-sulfonamide (23.70 mg) as a white solid. ¹H NMR (400 MHz, DMSO) δ 4.02-4.10 (2H, m), 4.59-4.67 (2H, m), 7.51-7.58 (6H, m), 7.63 (2H, s), 7.66 (1H, d), 7.84 (1H, dd), 7.97 (1H, d), 8.18 (1H, s). m/z (ES+) (M+H)+=446, 448

### Example 19

### 4-Amino-6-(2′-chloro-4′-(methylsulfonylmethyl)biphenyl-4-yl)-7,8-dihydropyrimido[5,4-f][1,4]oxazepin-5(6H)-one

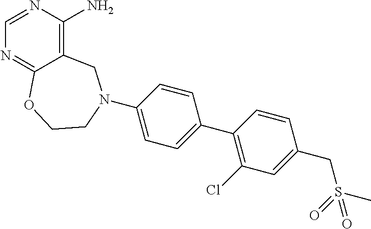

4-Amino-6-(4-bromophenyl)-7,8-dihydropyrimido[5,4-f][1,4]oxazepin-5(6H)-one (Intermediate 3, 0.06 g, 0.18 mmol), 2-(2-chloro-4-(methylsulfonylmethyl)phenyl)-4,4,5,5-tetramethyl-1,3,2-dioxaborolane (Intermediate 10; 0.089 g, 0.21 mmol), (1,1′-bis(diphenylphosphino)ferrocene)-dichloropalladium(II) (DCM adduct) (7.31 mg, 8.95 μmol) and tripotassium phosphate (0.046 g, 0.21 mmol) were suspended in DME (3 mL), methanol (1.500 mL) and water (0.750 mL) and sealed into a microwave tube. The mixture was degassed under vacuum and the atmosphere replaced with nitrogen. The reaction was heated to 110° C. for 40 minutes in the microwave reactor and cooled to RT. The reaction mixture was evaporated to dryness and redissolved in methyl THF (100 mL), and washed sequentially with water (100 mL) and saturated brine (100 mL). A solid was filtered off and MeOH/DCM added to this and re-filtered. The filtrate was combined with the organic layer from above, filtered through a phase separating funnel and evaporated to afford crude product. The crude product was purified by flash silica chromatography, elution gradient 1 to 6% MeOH in DCM, followed by repeat chromatography with elution gradient 1 to 8% MeOH in EtOAc. Pure fractions were evaporated to dryness to afford 4-amino-6-(2′-chloro-4′-(methylsulfonylmethyl)biphenyl-4-yl)-7,8-dihydropyrimido[5,4-f][1,4]oxazepin-5(6H)-one (6.30 mg, 7.67%) as a white solid.

¹H NMR (400 MHz, DMSO) δ 2.98 (3H, s), 3.97-4.06 (2H, m), 4.58 (2H, s), 4.62-4.68 (2H, m), 7.42-7.56 (6H, m), 7.62 (3H, s), 8.18 (1H, s). m/z (ES+) (M+H)+=459.31

### Example 20

### N-((4′-(4-amino-5-oxo-7,8-dihydropyrimido[5,4-f][1,4]oxazepin-6(5H)-yl)-2-chlorobiphenyl-4-yl)methyl)-N-methylmethanesulfonamide

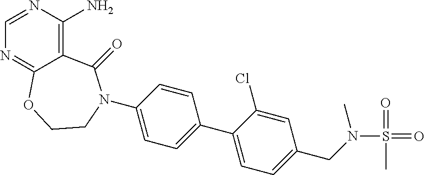

4-Amino-6-(4-bromophenyl)-7,8-dihydropyrimido[5,4-f][1,4]oxazepin-5(6H)-one (Intermediate 3; 0.13 g, 0.39 mmol), N-(3-chloro-4-(4,4,5,5-tetramethyl-1,3,2-dioxaborolan-2-yl)benzyl)-N-methylmethanesulfonamide (Intermediate 14; 0.167 g, 0.47 mmol), (1,1′-bis(diphenylphosphino)ferrocene)-dichloropalladium(II) (DCM adduct) (0.016 g, 0.02 mmol) and tripotassium phosphate (0.099 g, 0.47 mmol) were suspended in DME (3 mL), methanol (1.5 mL) and water (0.75 mL) and sealed into a microwave tube. The mixture was degassed under vacuum and the atmosphere replaced with nitrogen. The reaction was heated to 110° C. for 40 minutes in the microwave reactor and cooled to RT. The reaction mixture was evaporated to dryness and redissolved in methyl THF (100 mL), and washed sequentially with water (100 mL) and saturated brine (100 mL). A solid was filtered off and MeOH/DCM added to this and re-filtered. The filtrate was combined with the organic layer from above, filtered through a phase separation funnel and evaporated to afford crude product. The crude product was purified by preparative HPLC (Waters XBridge Prep C18 OBD column, 5μ silica, 50 mm diameter, 150 mm length), using decreasingly polar mixtures of water (containing 0.5% NH₃) and MeCN as eluents. Fractions containing the desired compound were evaporated to dryness to afford N-((4′-(4-amino-5-oxo-7,8-dihydropyrimido[5,4-f][1,4]oxazepin-6(5H)-yl)-2-chlorobiphenyl-4-yl)methyl)-N-methylmethanesulfonamide (0.046 g, 24.30%) as a white solid.

¹H NMR (400 MHz, DMSO) δ 2.74 (3H, s), 3.00 (3H, s), 4.03-4.08 (2H, m), 4.30 (2H, s), 4.60-4.66 (2H, m), 7.40 (1H, dd), 7.44-7.54 (6H, m), 7.62 (2H, s), 8.18 (1H, s). m/z (ES+) (M+H)+=488

### Example 21

### N-((4′-(4-amino-5-oxo-7,8-dihydropyrimido[5,4-f][1,4]oxazepin-6(5H)-yl)-2-chlorobiphenyl-4-yl)methyl)-N-methylacetamide

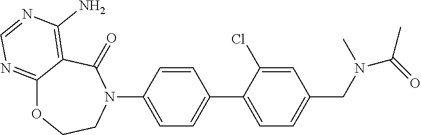

4-Amino-6-(4-bromophenyl)-7,8-dihydropyrimido[5,4-f][1,4]oxazepin-5(6H)-one (Intermediate 3; 0.19 g, 0.57 mmol), N-(3-chloro-4-(4,4,5,5-tetramethyl-1,3,2-dioxaborolan-2-yl)benzyl)-N-methylacetamide (Intermediate 18; 0.220 g, 0.68 mmol), (1,1′-bis(diphenylphosphino)ferrocene)-dichloropalladium(II) (DCM adduct) (0.023 g, 0.03 mmol) and tripotassium phosphate (0.144 g, 0.68 mmol) were suspended in DME (3 mL), methanol (1.5 mL) and water (0.75 mL) and sealed into a microwave tube. The mixture was degassed under vacuum and the atmosphere replaced with nitrogen. The reaction was heated to 110° C. for 40 minutes in the microwave reactor and cooled to RT. The reaction mixture was evaporated to dryness and redissolved in methyl THF (100 mL), and washed sequentially with water (100 mL) and saturated brine (100 mL). A solid was filtered off and MeOH/DCM added to this and re-filtered. The filtrate was combined with the organic layer from above, filtered through a phase separation funnel and evaporated to afford crude product. The crude product was purified by flash silica chromatography, elution gradient 1 to 8% MeOH in DCM. Pure fractions were evaporated to dryness to afford N-((4′-(4-amino-5-oxo-7,8-dihydropyrimido[5,4-f][1,4]oxazepin-6(5H)-yl)-2-chlorobiphenyl-4-yl)methyl)-N-methylacetamide (0.028 g, 10.93%) as a white solid.

¹H NMR (400 MHz, DMSO) δ 2.05-2.11 (3H, m), 2.91 (3H, d), 4.05 (2H, t), 4.53 (2H, s), 4.61-4.66 (2H, m), 7.27 (1H, t), 7.38-7.50 (6H, m), 7.62 (2H, s), 8.18 (1H, s). m/z (ES+) M+=452

### Example 22

### 4′-(4-Amino-5-oxo-7,8-dihydropyrimido[5,4-f][1,4]oxazepin-6(5H)-yl)-4-chlorobiphenyl-2-carbonitrile

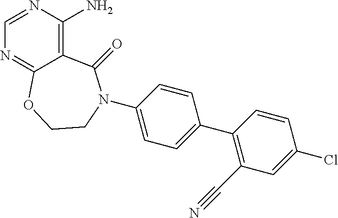

4-Amino-6-(4-(4,4,5,5-tetramethyl-1,3,2-dioxaborolan-2-yl)phenyl)-7,8-dihydropyrimido[5,4-f][1,4]oxazepin-5(6H)one (Intermediate 8; 250 mg, 0.65 mmol), 2-bromo-5-chlorobenzonitrile (156 mg, 0.72 mmol), (1,1′-bis(diphenylphosphino)ferrocene)-dichloropalladium(II) (DCM adduct) (26.7 mg, 0.03 mmol) and tripotassium phosphate (167 mg, 0.78 mmol) were suspended in DME (3 mL), ethanol (1.5 mL) and water (0.75 mL) and sealed into a microwave tube. The mixture was degassed under vacuum and the atmosphere replaced with nitrogen. The reaction was heated to 110° C. for 40 minutes in the microwave reactor and cooled to RT. The reaction mixture was diluted with methyl THF (60 mL), and washed sequentially with water (30 mL) and saturated brine (30 mL), filtered then evaporated to afford crude product. The crude product was purified by flash silica chromatography, elution gradient 1 to 6% MeOH in DCM. Pure fractions were evaporated to dryness and triturated with ether to afford 4′-(4-amino-5-oxo-7,8-dihydropyrimido[5,4-f][1,4]oxazepin-6(5H)-yl)-4-chlorobiphenyl-2-carbonitrile (113 mg, 44.1%) as a white solid. ¹H NMR (400 MHz, DMSO) δ 4.05-4.10 (2H, m), 4.60-4.66 (2H, m), 7.55-7.60 (2H, m), 7.63 (2H, s), 7.65-7.69 (3H, m), 7.88 (1H, dd), 8.15 (1H, d), 8.18 (1H, s). m/z (ES+) (M+H)+=392

### Example 23

### N-((4′-(4-amino-5-oxo-7,8-dihydropyrimido[5,4-f][1,4]oxazepin-6(5H)-yl)-2-chlorobiphenyl-4-yl)methyl)methanesulfonamide

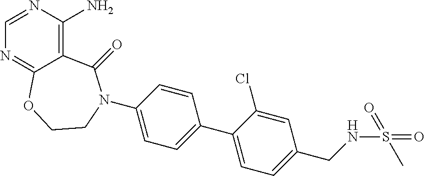

4-Amino-6-(4-bromophenyl)-7,8-dihydropyrimido[5,4-f][1,4]oxazepin-5(6H)-one (Intermediate 3; 244 mg, 0.73 mmol), N-(3-chloro-4-(4,4,5,5-tetramethyl-1,3,2-dioxaborolan-2-yl)benzyl)methanesulfonamide (Intermediate 20; 340 mg, 0.73 mmol), (1,1′-bis(diphenylphosphino)ferrocene)-dichloropalladium(II) (DCM adduct) (29.7 mg, 0.04 mmol) and tripotassium phosphate (185 mg, 0.87 mmol) were suspended in DME (3 mL), methanol (1.5 mL) and water (0.75 mL) and sealed into a microwave tube. The mixture was degassed under vacuum and the atmosphere replaced with nitrogen. The reaction was heated to 110° C. for 40 minutes in the microwave reactor and cooled to RT. The reaction mixture was evaporated to dryness and redissolved in methyl THF (100 mL), and washed sequentially with water (100 mL) and saturated brine (100 mL). A solid was filtered off and MeOH/DCM added to this and re-filtered. The filtrate was combined with the organic layer from above, filtered through a phase separation funnel and evaporated to afford crude product. The crude product was purified by preparative HPLC (Waters XBridge Prep C18 OBD column, 5μ silica, 50 mm diameter, 150 mm length), using decreasingly polar mixtures of water (containing 0.1% formic acid) and MeCN as eluents. Fractions containing the desired compound were evaporated and the residue further purified by HPLC using basic conditions (0.5% NH₃ in the water). Product containing fractions were evaporated to give N-((4′-(4-amino-5-oxo-7,8-dihydropyrimido[5,4-f][1,4]oxazepin-6(5H)-yl)-2-chlorobiphenyl-4-yl)methyl)methanesulfonamide (61.0 mg, 17.68%). ¹H NMR (400 MHz, DMSO) δ 2.94 (3H, s), 4.01-4.09 (2H, m), 4.22 (2H, d), 4.60-4.66 (2H, m), 7.37-7.45 (2H, m), 7.48 (4H, d), 7.55 (1H, s), 7.62 (3H, s), 8.18 (1H, s). m/z (ES+) (M+H)+=474

### Example 24

### N-((4′-(4-Amino-5-oxo-7,8-dihydropyrimido[5,4-f][1,4]oxazepin-6(5H)-yl)-2-chlorobiphenyl-4-yl)methybacetamide

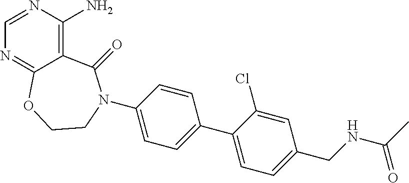

4-Amino-6-(4-bromophenyl)-7,8-dihydropyrimido[5,4-f][1,4]oxazepin-5(6H)-one (Intermediate 3; 0.248 g, 0.74 mmol), N-(3-chloro-4-(4,4,5,5-tetramethyl-1,3,2-dioxaborolan-2-yl)benzyl)acetamide (Intermediate 22; 0.27 g, 0.74 mmol), (1,1′-bis(diphenylphosphino)ferrocene)-dichloropalladium(II) (DCM adduct) (0.030 g, 0.04 mmol) and tripotassium phosphate (0.189 g, 0.89 mmol) were suspended in DME (3 mL), methanol (1.5 mL) and water (0.75 mL) and sealed into a microwave tube. The mixture was degassed under vacuum and the atmosphere replaced with nitrogen. The reaction was heated to 110° C. for 40 minutes in the microwave reactor and cooled to RT. The reaction mixture was evaporated to dryness and redissolved in methyl THF (100 mL), and washed sequentially with water (100 mL) and saturated brine (100 mL). A solid was filtered off and MeOH/DCM added to this and re-filtered. The filtrate was combined with the organic layer from above, filtered through a phase separating funnel and evaporated to afford crude product. The crude product was purified by preparative HPLC (Waters XBridge Prep C18 OBD column, 5μ silica, 50 mm diameter, 150 mm length), using decreasingly polar mixtures of water (containing 0.5% NH₃) and MeCN as eluents. Fractions containing the desired compound were evaporated to dryness to afford N-((4′-(4-amino-5-oxo-7,8-dihydropyrimido[5,4-f][1,4]oxazepin-6(5H)-yl)-2-chlorobiphenyl-4-yl)methyl)acetamide (0.080 g, 24.65%) as a colourless solid. ¹H NMR (400 MHz, DMSO) δ 1.83 (3H, s), 3.95-4.01 (2H, m), 4.22 (2H, d), 4.53-4.59 (2H, m), 7.23 (1H, dd), 7.32 (1H, d), 7.37 (1H, d), 7.41 (4H, s), 7.55 (2H, s), 8.11 (1H, s), 8.34 (1H, t). m/z (ES+) (M+H)+=438

### Examples 25-30

Prepared following the method of example 5, coupling Intermediate 3 with the appropriate commercially available boronic acid or synthesized intermediate where indicated.

| Example | Structure | ¹H NMR (400 MHz) | Mass Spec |
|---|---|---|---|
| 25 4-Amino-6-(2′,3′- difluorobiphenyl-4- yl)-7,8- dihydropyrimido[5,4- f][1,4]oxazepin- 5(6H)-one | 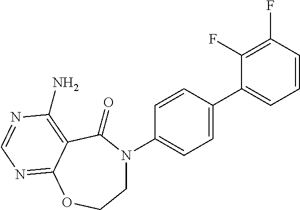 | (CDCl₃) δ 4.08 (2H, dd), 4.66-4.79 (2H, m), 5.64 (1H, s), 7.11- 7.22 (3H, m), 7.36 7.42 (2H, m), 7.61-7.67 (2H, m), 8.18 (1H, s), 8.30 (1H, s). | m/z (ES+) (M + H)+ = 369 |
| 26 4-Amino-6-(2′,5′- difluorobiphenyl-4- yl)-7,8- dihydropyrimido[5,4- f][1,4]oxazepin- 5(6H)-one | 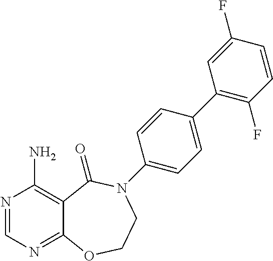 | (DMSO) δ 3.98-4.09 (2H, m), 4.58-4.69 (2H, m), 7.20-7.32 (1H, m), 7.33-7.48 (2H, m), 7.48-7.55 (2H, m), 7.58-7.70 (4H, m), 8.18 (1H, s). | m/z (ES+) (M + H)+ = 369 |
| 27 4-Amino-6-(4′-chloro- 2′-fluorobiphenyl-4- yl)-7,8- dihydropyrimido[5,4- f][1,4]oxazepin- 5(6H)-one | 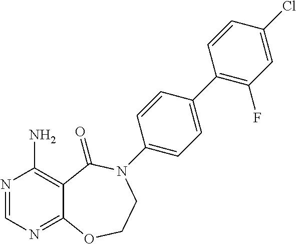 | (DMSO) δ 4.05 (2H, dd), 4.58-4.67 (2H, m), 7.37-7.44 (1H, m), 7.46-7.70 (8H, m), 8.18 (1H, s). | m/z (ES+) (M + H)+ = 385 |
| 28 4-Amino-6-(2′,4′,5′- trifluorobiphenyl-4- yl)-7,8- dihydropyrimido[5,4- f][1,4]oxazepin- 5(6H)-one | 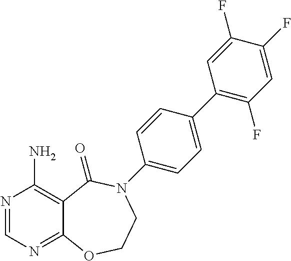 | (DMSO) δ 3.95-4.11 (2H, m), 4.56-4.68 (2H, m), 7.44-7.55 (2H, m), 7.57-7.81 (6H, m), 8.18 (1H, s). | m/z (ES+) (M + H)+ = 387 |
| 29 4-Amino-6-(2′,4′- difluorobiphenyl-4- yl)-7,8- dihydropyrimido[5,4- f][1,4]oxazepin- 5(6H)-one | 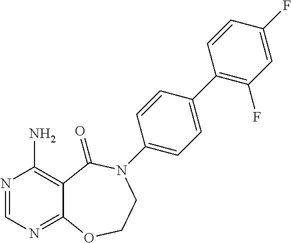 | (DMSO) δ 3.97-4.10 (2H, m), 4.57-4.70 (2H, m), 7.21 (1H, ddd), 7.32-7.42 (1H, m), 7.45-7.53 (2H, m), 7.54-7.67 (5H, m), 8.18 (1H, s). | m/z (ES+) (M + H)+ = 369 |
| 30* 4-Amino-6-(2′-chloro- 4′-((2-oxopyrimidin- 1(2H)- yl)methyl)biphenyl-4- yl)-7,8- dihydropyrimido[5,4- f][1,4]oxazepin- 5(6H)-one | 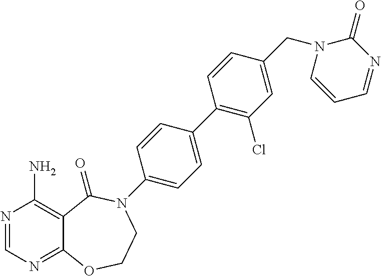 | (CDCl₃) δ 4.06 4.10 (2H, m), 4.70-4.77 (2H, m), 5.11 (2H, s), 5.53-5.79 (1H, m), 6.34 (1H, dd), 7.30- 7.38 (4H, m), 7.47 (1H, t), 7.49-7.54 (2H, m), 7.69 (1H, dd), 8.03- 8.24 (1H, m), 8.30 (1H, s), 8.63 (1H, dd). | m/z (ES+) (M + H)+ = 475 |

*Intermediate 3 coupled with intermediate 47a

### Examples 31-37

Prepared by the method of Example 22 by coupling of Intermediate 8 to the appropriate commercial or synthesized intermediate as indicated

| | | ¹H NMR (400 | Mass | Coupling |
|---|---|---|---|---|
| Example | Structure | MHz) | Spec | partner |
| 31 4-Amino-6-(2′-chloro-4′- (difluoromethyl)biphenyl- 4-yl)-7,8- dihydropyrimido[5,4- f][1,4]oxazepin-5(6H)- one | 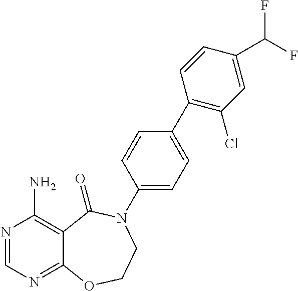 | (DMSO) δ 4.03- 4.11 (2H, m), 4.58- 4.68 (2H, m), 7.10 (1H, t), 7.49 - 7.67 (8H, m), 7.79 (1H, s), 8.18 (1H, s). | m/z (ES+) (M + H)+ = 417 | Intermediate 24 |
| 32 4′-(4-Amino-5-oxo-7,8- dihydropyrimido[5,4- f][1,4]oxazepin-6(5H)- yl)-2-chlorobiphenyl-4- carbonitrile | 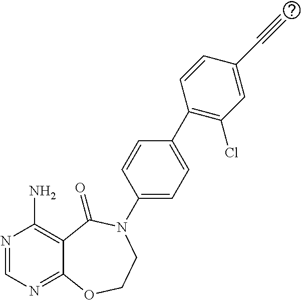 | (CDCl₃) 4.07- 4.11 (2H, m), 4.72- 4.76 (2H, m), 5.69 (1H, s), 7.39- 7.42 (2H, m), 7.47 (1H, d), 7.51- 7.56 (2H, m), 7.63 (1H, dd), 7.80 (1H, d), 8.15 (1H, s), 8.31 (1H, s). | m/z (ES+) (M + H)+ = 392 | 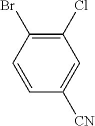 |
| 33 4′-(4-Amino-5-oxo-7,8- dihydropyrimido[5,4- f][1,4]oxazepin-6(5H)- yl)-2-chlorobiphenyl-3- carbonitrile | 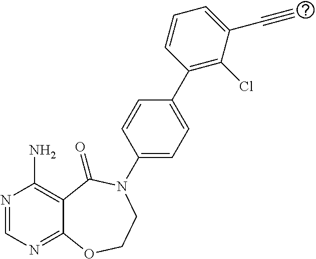 | (DMSO) δ 4.03- 4.08 (2H, m), 4.60- 4.66 (2H, m), 7.53 (4H, s), 7.59- 7.67 (3H, m), 7.79 (1H, dd), 8.01 (1H, dd), 8.18 (1H, s). | m/z (ES+) (M + H)+ = 392 | Intermediate 25 |
| 34 4′-(4-Amino-5-oxo-7,8- dihydropyrimido[5,4- f][1,4]oxazepin-6(5H)- yl)-6-chlorobiphenyl-2- carbonitrile | 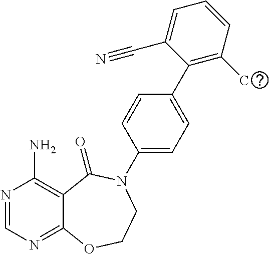 | (DMSO) δ 4.03- 4.13 (2H, m), 4.62- 4.69 (2H, m), 7.46-7.52 (2H, m), 7.55-7.66 (5H, m), 7.96 (2H, ddd), 8.18 (1H, s). | m/z (ES+) (M + H)+ = 392 | Intermediate 26 |
| 36 4-Amino-6-(2′-chloro-4′- (hydroxymethyl)biphenyl- 4-yl)-7,8- dihydropyrimido[5,4- f][1,4]oxazepin-5(6H)- one | 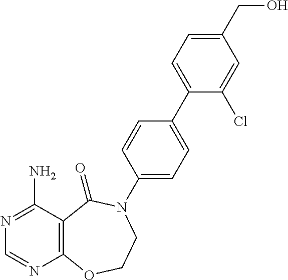 | (DMSO) δ 4.06 (2H, dd), 4.55 (2H, d), 4.58 4.68 (2H, m), 5.34 (1H, t), 7.32 7.43 (2H, m), 7.45 7.54 (5H, m), 7.62 (2H, s), 8.18 (1H, s). | m/z (ES+) (M + H)+ = 397 | 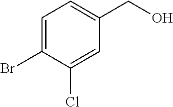 |
| 37 4-Amino-6-(2′-chloro- 6′-methylbiphenyl-4-yl)- 7,8-dihydropyrimido[5,4- f][1,4]oxazepin-5(6H)- one | 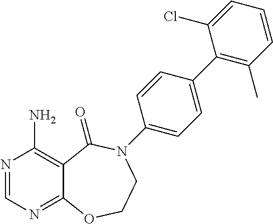 | (DMSO) δ 2.07 (3H, s), 3.99- 4.12 (2H, m), 4.64 (2H, dd), 7.21- 7.33 (4H, m), 7.35- 7.44 (1H, m), 7.44-7.53 (2H, m), 7.63 (2H, s), 8.18 (1H, s). | m/z (ES+) (M + H)+ = 381.28 | 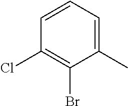 |

There is no example with Example number 35.

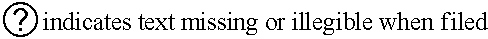

### Example 38

### (S)-4-amino-6-(2′-chloro-4′-((3-hydroxy-2-oxopyrrolidin-1-yl)methyl)biphenyl-4-yl)-7,8-dihydropyrimido[5,4-f][1,4]oxazepin-5(6H)-one

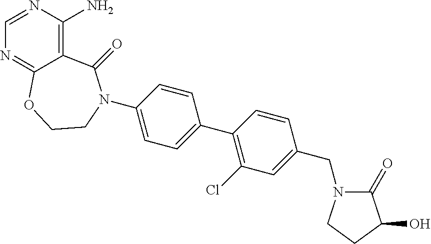

4M HCl in dioxane (5 mL) was added to (S)-4-amino-6-(4′-((3-(tert-butyldimethylsilyloxy)-2-oxopyrrolidin-1-yl)methyl)-2′-chlorobiphenyl-4-yl)-7,8-dihydropyrimido[5,4-f][1,4]oxazepin-5(6H)-one (Intermediate 27; 70 mg, 0.09 mmol) and stirred at RT for 1 hour. The reaction mixture was evaporated to dryness and redissolved in DCM (150 mL), and washed sequentially with saturated NaHCO₃ (100 mL) and saturated brine (100 mL). The organic layer was separated and filtered through a phase separating funnel and evaporated to afford an orange gum. The crude product was purified by flash silica chromatography, elution gradient 1 to 10% DCM in MeOH. Pure fractions were evaporated to dryness to afford the title compound (33.0 mg, 78%) as a white solid.

¹H NMR (DMSO) δ 2.29 (2H, d), 3.10-3.20 (2H, m), 3.99-4.08 (2H, m), 4.17 (1H, t), 4.40 (2H, q), 4.55-4.65 (2H, m), 5.49-5.63 (1H, m), 7.26 (1H, d), 7.40 (2H, d), 7.47 (4H, s), 7.61 (2H, s), 8.16 (1H, s). m/z (ES+) (M+H)+=480

### Example 39

### 3-Amino-N-((4′-(4-amino-5-oxo-7,8-dihydropyrimido[5,4-f][1,4]oxazepin-6(5H)-yl)-2-chlorobiphenyl-4-yl)methyl)-N-methylpropanamide

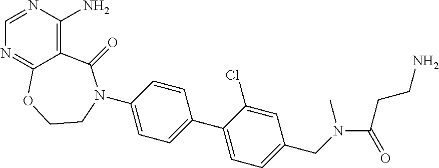

Trifluoroacetic acid (0.292 mL, 3.79 mmol) was added to tert-butyl 3-(((4′-(4-amino-5-oxo-7,8-dihydropyrimido[5,4-f][1,4]oxazepin-6(5H)-yl)-2-chlorobiphenyl-4-yl)methyl)(methyl)amino)-3-oxopropylcarbamate (Intermediate 31; 0.22 g, 0.38 mmol) in DCM (10 mL) at 20° C. The resulting solution was stirred at 20° C. for 24 hours. The reaction mixture was evaporated. The crude product was purified by ion exchange chromatography, using an SCX column. The desired product was eluted from the column using 7M NH₃/MeOH and pure fractions were evaporated to dryness. The crude gum was triturated with hot EtOH to give a solid which was collected by filtration and dried under vacuum to give the title compound (0.098 g, 53.8%) as a white solid. ¹H NMR (400 MHz, DMSO) 1.75 (2H, s), 2.45 (2H, dd), 2.73-2.81 (2H, m), 2.91 (3H, d), 4.01-4.07 (2H, m), 4.54-4.62 (2H, m), 4.64 (2H, d), 7.26 (1H, t), 7.37-7.42 (2H, m), 7.44-7.51 (4H, m), 7.62 (2H, s), 8.17 (1H, d). m/z (ES+) (M+H)+=481

### Example 40

### 2-Amino-N-((4′-(4-amino-5-oxo-7,8-dihydropyrimido[5,4-f][1,4]oxazepin-6(5H)-yl)-2-chlorobiphenyl-4-yl)methyl)-N-methylacetamide

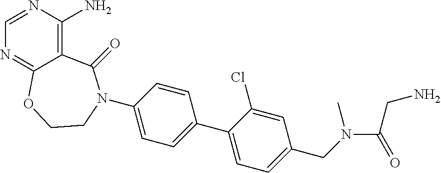

Prepared from Intermediate 34 by the method of Example 39. ¹H NMR (400 MHz, DMSO) 1.68 (2H, s), 2.90 (3H, d), 3.39 (2H, d), 4.02-4.06 (2H, m), 4.56 (2H, s), 4.60-4.66 (2H, m), 7.22-7.30 (1H, m), 7.37-7.43 (2H, m), 7.47 (4H, d), 7.62 (2H, s), 8.17 (1H, d). m/z (ES+) (M+H)+=467

### Example 41

### 4-amino-N-((4′-(4-amino-5-oxo-7,8-dihydropyrimido[5,4-f][1,4]oxazepin-6(5H)-yl)-2-chlorobiphenyl-4-yl)methyl)-N-methylbutanamide

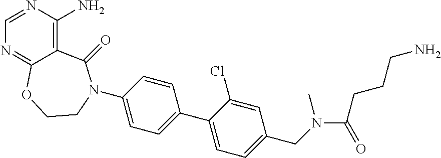

Prepared from Intermediate 37 by the method of Example 39. ¹H NMR (400 MHz, DMSO, 100° C.) 1.44-1.57 (2H, m), 1.63-1.72 (2H, m), 2.43 (2H, t), 2.62 (2H, t), 2.92 (3H, s), 4.00-4.08 (2H, m), 4.58 (2H, s), 4.62-4.67 (2H, m), 7.27 (1H, d), 7.36-7.55 (8H, m), 8.17 (1H, s). m/z (ES+) (M+H)+=495

### Example 42

### 4-Amino-6-(2′-chlorobiphenyl-4-yl)-2-methyl-7,8-dihydropyrimido[5,4-f][1,4]oxazepin-5(6H)-one

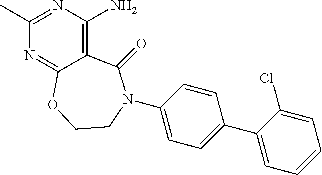

4-Amino-6-(4-bromophenyl)-2-methyl-7,8-dihydropyrimido[5,4-f][1,4]oxazepin-5(6H)-one (Intermediate 40; 0.15 g, 0.43 mmol), 2-chlorophenylboronic acid (0.074 g, 0.47 mmol), (1,1′-bis(diphenylphosphino)ferrocene)-dichloropalladium(II) (DCM adduct) (0.018 g, 0.02 mmol) and tripotassium phosphate (0.109 g, 0.52 mmol) were suspended in DME (3 mL), methanol (1.5 mL) and water (0.75 mL) and sealed into a microwave tube. The mixture was degassed under vacuum and the atmosphere replaced with nitrogen. The reaction was heated to 110° C. for 40 minutes in the microwave reactor and cooled to RT. The reaction mixture was evaporated to dryness and redissolved in methyl THF (100 mL), and washed sequentially with water (100 mL) and saturated brine (100 mL). A solid was filtered off and MeOH/DCM added to this and re-filtered. The filtrate was combined with the organic layer from above, filtered through a phase separating funnel and evaporated to afford crude product. The crude product was purified by flash silica chromatography, elution gradient 1 to 8% MeOH in DCM. Pure fractions were evaporated to dryness to afford a colourless oil. The crude oil was triturated with Et₂O to give a solid which was collected by filtration and dried under vacuum to give the title compound (0.045 g, 27.5%) as a white solid. ¹H NMR (400 MHz, CDCl₃) 2.46 (3H, s), 4.08 (2H, dd), 4.70-4.75 (2H, m), 5.62 (1H, s), 7.28-7.37 (5H, m), 7.46-7.50 (1H, m), 7.52-7.56 (2H, m), 8.21 (1H, s). m/z (ES+) (M+H)+=381

### Example 43

### N-((4′-(4-amino-2-methyl-5-oxo-7,8-dihydropyrimido[5,4-f][1,4]oxazepin-6(5H)-yl)-2-chlorobiphenyl-4-yl)methyl)methanesulfonamide

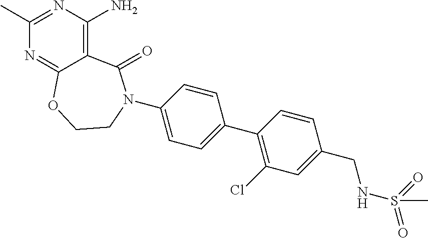

Prepared by the method of Example 42 by coupling of Intermediate 40 with Intermediate 20. ¹H NMR (400 MHz, DMSO) 2.28 (3H, s), 2.93 (3H, s), 4.00-4.05 (2H, m), 4.22 (2H, d), 4.56-4.62 (2H, m), 7.34-7.68 (10H, m). m/z (ES+) (M+H)+=488.35

### Example 44

### (R)-4-Amino-6-(2′-chlorobiphenyl-4-yl)-8-methyl-7,8-dihydropyrimido[5,4-f][1,4]oxazepin-5(6H)-one

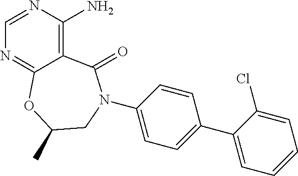

(R)-4-(4-Amino-8-methyl-5-oxo-7,8-dihydropyrimido[5,4-f][1,4]oxazepin-6(5H)-yl)phenyl trifluoromethanesulfonate (Intermediate 48; 0.23 g, 0.55 mmol), 2-chlorophenylboronic acid (0.120 g, 0.77 mmol), (1,1′-bis(diphenylphosphino)ferrocene)-dichloropalladium(II) (DCM adduct) (0.022 g, 0.03 mmol) and tripotassium phosphate (0.140 g, 0.66 mmol) were suspended in DME (3 mL), methanol (1.500 mL) and water (0.750 mL) and sealed into a microwave tube. The mixture was degassed under vacuum and the atmosphere replaced with nitrogen. The reaction was heated to 110° C. for 40 minutes in the microwave reactor and cooled to RT. The reaction mixture was evaporated to dryness and redissolved in methyl THF (100 mL), and washed sequentially with water (100 mL) and saturated brine (100 mL). A solid was filtered off and MeOH/DCM added to this and re-filtered. The filtrate was combined with the organic layer from above, filtered through a phase separating funnel and evaporated to afford crude product. Purified by preparative HPLC (Phenomenex Gemini C18 110A (axia) column, 5 g silica, 30 mm diameter, 100 mm length), using decreasingly polar mixtures of water (containing 0.5% formic acid) and MeCN as eluents to afford the title compound (0.114 g, 54.4%) as a white solid. ¹H NMR (400 MHz, CDCl₃) 1.51 (3H, d), 3.89-4.00 (2H, m), 5.00 (1H, ddd), 5.65 (1H, s), 7.28 7.41 (5H, m), 7.46-7.51 (1H, m), 7.52-7.59 (2H, m), 8.04 (1H, s), 8.32 (1H, s). m/z (ES+) (M+H)+=381

### Example 45

### (R)—N-((4′-(4-amino-8-methyl-5-oxo-7,8-dihydropyrimido[5,4-f][1,4]oxazepin-6(5H)-yl)-2-chlorobiphenyl-4-yl)methyl)methanesulfonamide

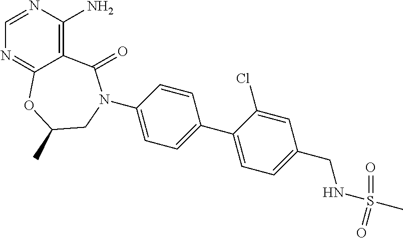

Prepared by the method of Example 44 by coupling of Intermediate 48 with Intermediate 20. ¹H NMR (400 MHz, CDCl₃) 1.50 (3H, d), 2.99 (3H, s), 3.83-4.01 (2H, m), 4.37 (2H, d), 4.64 (1H, s), 4.98 (1H, td), 5.48 5.76 (1H, m), 7.31-7.40 (4H, m), 7.48-7.57 (3H, m), 7.85-8.13 (1H, m), 8.32 (1H, s). m/z (ES+) (M+H)+=488

### Example 46

### (R)-4-amino-6-(2′-fluorobiphenyl-4-yl)-8-methyl-7,8-dihydropyrimido[5,4-f][1,4]oxazepin-5(6H)-one

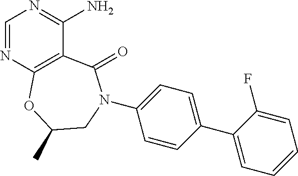

Prepared by the method of Example 44 by coupling of Intermediate 48 with 2-fluorophenylboronic acid. ¹H NMR (400 MHz, CDCl₃, 30° C.) 1.50 (3H, d), 3.83-4.04 (2H, m), 4.98 (1H, qt), 5.69 (1H, s), 7.13-7.22 (1H, m), 7.25 (1H, d), 7.31-7.36 (1H, m), 7.36-7.40 (2H, m), 7.41-7.48 (1H, m), 7.65 (2H, dd), 8.06 (1H, s), 8.32 (1H, s). m/z (ES+) (M+H)+=365

### Example 47

### (R)—N-((4′-(4-amino-8-methyl-5-oxo-7,8-dihydropyrimido[5,4-f][1,4]oxazepin-6(5H)-yl)-2-chlorobiphenyl-4-yl)methyl)-N-methylmethanesulfonamide

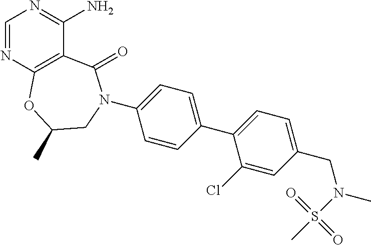

Prepared by the method of Example 44 by coupling of Intermediate 48 with Intermediate 14. ¹H NMR (400 MHz, CDCl₃, 30° C.) 1.51 (3H, d), 2.84 (3H, s), 2.90 (3H, s), 3.88 4.01 (2H, m), 4.34 (2H, s), 4.94 5.03 (1H, m), 5.53 5.87 (1H, m), 7.31 7.40 (4H, m), 7.48 (1H, s), 7.51 7.57 (2H, m), 8.02 (1H, s), 8.32 (1H, s). m/z (ES+), (M+H)+=502

### Example 48

### (R)-4-Amino-6-(2′-chloro-4′-(methylsulfonylmethyl)biphenyl-4-yl)-8-methyl-7,8-dihydropyrimido[5,4-f][1,4]oxazepin-5(6H)-one

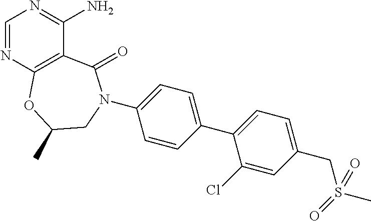

A solution of sodium tungstate dihydrate (3.29 mg, 9.98 μmol) in water (0.5 mL) was added to a stirred solution of (R)-4-amino-6-(2′-chloro-4′-(methylthiomethyl)biphenyl-4-yl)-8-methyl-7,8-dihydropyrimido[5,4-f][1,4]oxazepin-5(6H)-one (Intermediate 49; 220 mg, 0.50 mmol) in methanol (5 mL) and DCM (5.00 mL). The mixture was cooled to 0° C., 3-chloroperoxybenzoic acid (246 mg, 1.10 mmol) was added, and the resulting mixture was stirred at 20° C. for 4 hours. The reaction was incomplete and further 3-chloroperoxybenzoic acid (30 mg), was added and the solution was stirred at 20° C. for a further 16 hours. The reaction mixture was diluted with saturated NaHCO₃ (12 mL), and evaporated to remove the organic layer. The aqueous layer was extracted with methyl THF (125 mL) and a beige solid filtered off. The filtrate was evaporated and triturated with hot MeOH to give a solid which was collected by filtration, slurried in acetonitrile and stirred overnight, then filtered and dried under vacuum to give the title compound (76 mg, 32.2%) as a beige solid. ¹H NMR (400 MHz, CDCl₃, 30° C.) 1.51 (3H, d), 2.88 (3H, s), 3.89-4.01 (2H, m), 4.27 (2H, s), 4.94-5.03 (1H, m), 5.62 (1H, s), 7.39 (4H, dd), 7.54 (3H, dd), 8.01 (1H, s), 8.32 (1H, s). m/z (ES+), (M+H)+=473

### Example 49

### (R)-4-amino-6-(2′,6′-difluorobiphenyl-4-yl)-8-methyl-7,8-dihydropyrimido[5,4-f][1,4]oxazepin-5(6H)-one

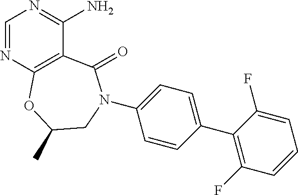

0.5M Ammonia in 1,4-dioxane (10 mL) was added in one portion to (R)-4-chloro-6-(2′,6′-difluorobiphenyl-4-yl)-8-methyl-7,8-dihydropyrimido[5,4-f][1,4]oxazepin-5(6H)-one (Intermediate 50; 0.053 g, 0.13 mmol) at 20° C. The resulting solution was stirred at 55° C. for 4 hours. The reaction mixture was evaporated. The crude product was purified by flash silica chromatography, elution gradient 1 to 7% MeOH in DCM. Pure fractions were evaporated to dryness to afford the title compound (0.011 g, 21.81%) as a white solid.

¹H NMR (400 MHz, CDCl₃) 1.50 (3H, d), 3.88-3.98 (2H, m), 4.97 (1H, pd), 5.64 (1H, s), 6.97-7.05 (2H, m), 7.30 (1H, ddd), 7.36-7.43 (2H, m), 7.53-7.62 (2H, m), 8.02 (1H, s), 8.32 (1H, s). m/z (ES+) (M+H)+=383

### Example 50

### (R)-4′-(4-Amino-8-methyl-5-oxo-7,8-dihydropyrimido[5,4-f][1,4]oxazepin-6(5H)-yl)-4-chloro-2′-fluorobiphenyl-2-carbonitrile

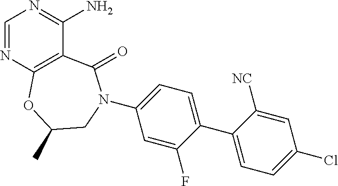

(R)-4-Amino-6-(3-fluoro-4-(4,4,5,5-tetramethyl-1,3,2-dioxaborolan-2-yl)phenyl)-8-methyl-7,8-dihydropyrimido[5,4-f][1,4]oxazepin-5(6H)-one (Intermediate 51; 290 mg, 0.70 mmol), PdCl₂(dppf)-DCM adduct (28.6 mg, 0.04 mmol), potassium phosphate (0.07 mL, 0.84 mmol) and 2-bromo-5-chlorobenzonitrile (182 mg, 0.84 mmol) were suspended in DME (3 mL), ethanol (1.5 mL) and water (0.75 mL) and sealed into a microwave tube, degassed under vacuum and the atmosphere replaced with nitrogen. The reaction was heated to 130° C. for 90 minutes in the microwave reactor and cooled to RT. The reaction mixture was evaporated to dryness and redissolved in methyl THF (25 mL), and washed sequentially with water (25 mL) and saturated brine (25 mL). The organic layer was dried over Na₂SO₄, filtered and evaporated to afford crude product. The crude product was purified by preparative HPLC (Waters XBridge Prep C18 OBD column, 5μ silica, 50 mm diameter, 150 mm length), using decreasingly polar mixtures of water (containing 0.1% formic acid) and MeCN as eluents. Fractions containing the desired compound were evaporated to dryness to afford the title compound (74.0 mg, 24.94%) as a white solid.

¹H NMR (400 MHz, CDCl₃, 30° C.) 2.61 (3H, s), 3.89-4.00 (2H, m), 4.86-5.08 (1H, m), 5.51-5.74 (1H, m), 7.22 (1H, d), 7.49 (2H, dd), 7.66 (1H, dd), 7.78 (1H, d), 7.82-8.07 (1H, m), 8.33 (1H, s). m/z (ES+), (M+H)+=424

### Example 51

### (R)-4′-(4-Amino-8-methyl-5-oxo-7,8-dihydropyrimido[5,4-f][1,4]oxazepin-6(5H)-yl)-2-chloro-2′-fluorobiphenyl-4-carbonitrile

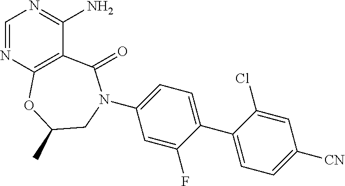

Prepared by the method of Example 50 by coupling of Intermediate 51 with 4-bromo-3-chlorobenzonitrile. ¹H NMR (400 MHz, CDCl₃, 30° C.) 1.26 (3H, d), 3.88-4.00 (2H, m), 4.93-5.01 (1H, m), 5.50-5.75 (1H, m), 7.19-7.24 (2H, m), 7.40 (1H, t), 7.47 (1H, d), 7.64 (1H, dd), 7.81 (1H, d), 7.85-8.05 (1H, m), 8.33 (1H, s). m/z (ES+), (M+H)+=424

### Example 52

### 4′-((R)-4-Amino-8-methyl-5-oxo-7,8-dihydropyrimido[5,4-f][1,4]oxazepin-6(5H)-yl)-6-chloro-2′-fluorobiphenyl-2-carbonitrile

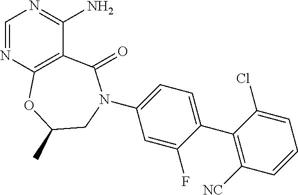

(R)-4-Amino-6-(3-fluoro-4-(4,4,5,5-tetramethyl-1,3,2-dioxaborolan-2-yl)phenyl)-8-methyl-7,8-dihydropyrimido[5,4-f][1,4]oxazepin-5(6H)one (Intermediate 51; 290 mg, 0.70 mmol), PdCl₂(dppf)-DCM adduct (28.6 mg, 0.04 mmol), potassium phosphate (0.070 mL, 0.84 mmol) and 2-chloro-6-cyanophenyl trifluoromethanesulfonate (240 mg, 0.84 mmol) were suspended in DME (3 mL), ethanol (1.5 mL) and water (0.75 mL) and sealed into a microwave tube, degassed under vacuum and the atmosphere replaced with nitrogen. The reaction was heated to 130° C. for 90 minutes in the microwave reactor and cooled to RT. The reaction mixture was evaporated to dryness and redissolved in methyl THF (25 mL), and washed sequentially with water (25 mL) and saturated brine (25 mL). The organic layer was dried over Na₂SO₄, filtered and evaporated to afford crude product. The crude product was purified by basic preparative HPLC (Waters XBridge Prep C18 OBD column, 5μ silica, 50 mm diameter, 150 mm length), using decreasingly polar mixtures of water (containing 0.5% ammonia) and MeCN as eluents. Fractions containing the desired compound were evaporated to dryness to afford the title compound (48.7 mg, 16.41%) as a white solid. The reaction was repeated on 1.03× scale giving a further crop of the title compound (41.4 mg) which was combined with the first.

¹H NMR (400 MHz, CDCl₃, 30° C.) 1.52 (3H, d), 3.93-3.98 (2H, m), 4.93-5.03 (1H, m), 5.64 (1H, s), 7.24-7.29 (2H, m), 7.39-7.53 (2H, m), 7.68-7.79 (2H, m), 7.98 (1H, s), 8.33 (1H, s). m/z (ES+), (M+H)+=424

### Example 53

### (R)-4-Amino-8-methyl-6-(2,2′,4′-trifluorobiphenyl-4-yl)-7,8-dihydropyrimido[5,4-f][1,4]oxazepin-5(6H)-one

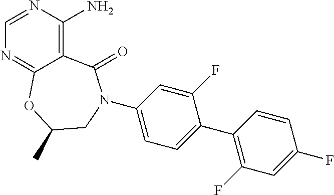

(R)-4-(4-Amino-8-methyl-5-oxo-7,8-dihydropyrimido[5,4-f][1,4]oxazepin-6(5H)-yl)-2-fluorophenyl trifluoromethanesulfonate (Intermediate 51a; 0.3 g, 0.69 mmol), 2,4-difluorophenylboronic acid (0.163 g, 1.03 mmol), (1,1′-bis(diphenylphosphino)ferrocene)-dichloropalladium(II) (DCM adduct) (0.028 g, 0.03 mmol) and tripotassium phosphate (0.175 g, 0.83 mmol) were suspended in DME (3 mL), ethanol (1.5 mL) and water (0.75 mL) and sealed into a microwave tube. The mixture was degassed under vacuum and the atmosphere replaced with nitrogen. The reaction was heated to 110° C. for 60 minutes in the microwave reactor and cooled to RT. The reaction mixture was evaporated to dryness and redissolved in methyl THF (100 mL), and washed sequentially with water (100 mL) and saturated brine (100 mL). The organic layer was dried over MgSO₄, filtered and evaporated. The crude product was purified by preparative HPLC (Phenomenex Gemini C18 110A (axia) column, 5μ silica, 30 mm diameter, 100 mm length), using decreasingly polar mixtures of water (containing 0.5% formic acid) and MeCN as eluents. Fractions containing the desired compound were evaporated to dryness to afford a solid which was slurried overnight in acetonitrile to give the title compound (0.102 g, 37.1%) as a white solid. ¹H NMR (400 MHz, CDCl₃) 1.51 (3H, d), 3.90-3.95 (2H, m), 4.92-5.01 (1H, m), 5.65 (1H, s), 6.91-7.04 (2H, m), 7.15-7.21 (2H, m), 7.37 (1H, dd), 7.45 (1H, t), 8.04 (1H, s), 8.33 (1H, s). m/z (ES+) (M+H)+=401

### Example 54

### (R)-4-Amino-6-(2,2′-difluorobiphenyl-4-yl)-8-methyl-7,8-dihydropyrimido[5,4-f][1,4]oxazepin-5(6H)-one

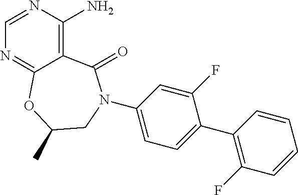

Prepared by the method of Example 53 by coupling of Intermediate 51a with 2-fluorophenylboronic acid. ¹H NMR (400 MHz, CDCl₃) δ 1.51 (3H, d), 3.87-4.00 (2H, m), 4.93-5.02 (1H, m), 5.64 (1H, s), 7.14-7.23 (3H, m), 7.22-7.25 (1H, m), 7.35-7.44 (2H, m), 7.49 (1H, t), 8.03 (1H, s), 8.33 (1H, s). m/z (ES+) (M+H)+=383

### Example 55

### (R)-4-Amino-6-(2′-chloro-2-fluorobiphenyl-4-yl)-8-methyl-7,8-dihydropyrimido[5,4-f][1,4]oxazepin-5(6H)-one

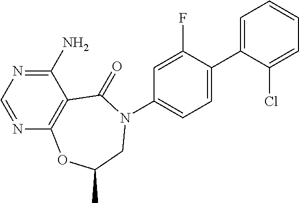

(R)-4-(4-Amino-8-methyl-5-oxo-7,8-dihydropyrimido[5,4-f][1,4]oxazepin-6(5H)-yl)-2-fluorophenyl trifluoromethanesulfonate (Intermediate 51a; 160 mg, 0.37 mmol), 2-chlorophenylboronic acid (57.3 mg, 0.37 mmol) and tripotassium phosphate (93 mg, 0.44 mmol) were suspended in DME (3 mL), water (0.75 mL) and ethanol (1.5 mL) and degassed. (1,1′-Bis(diphenylphosphino)ferrocene)-dichloropalladium(II) (DCM adduct) (24.13 mg, 0.03 mmol) was added and the suspension was sealed into a microwave tube. The reaction was heated to 110° C. for 60 minutes in the microwave reactor and cooled to RT. The suspension was poured directly onto a SCX column and the crude reaction mixture was purified by ion exchange chromatography, using an SCX column. The column was first washed with methanol (50 mL) and the desired product was then eluted from the column using 0.35M NH₃/MeOH. Fractions containing the required product were evaporated to dryness and redissolved in DCM (15 mL), and washed sequentially with saturated NaHCO₃ (15 mL). The organic layer was dried using an isolute phase separating column and evaporated to afford the title product (72.0 mg, 49.2%) as a tan solid. ¹H NMR (400 MHz, DMSO, 30° C.) δ 1.28 (3H, d), 3.85-3.95 (1H, m), 3.96-4.06 (1H, m), 4.89-5.02 (1H, m), 7.30-7.53 (7H, m), 7.56-7.65 (2H, m), 8.17-8.26 (1H, m). m/z (ES+) (M+H)+=399

### Example 56

### (8R)-4-amino-6-(2′-chloro-2,6′-difluorobiphenyl-4-yl)-8-methyl-7,8-dihydropyrimido[5,4-f][1,4]oxazepin-5(6H)-one

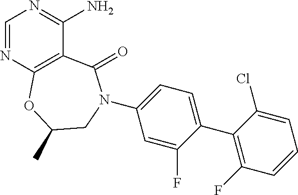

(R)-4-(4-Amino-8-methyl-5-oxo-7,8-dihydropyrimido[5,4-f][1,4]oxazepin-6(5H)-yl)-2-fluorophenyl trifluoromethanesulfonate (Intermediate 51a; 300 mg, 0.69 mmol), potassium (2-chloro-6-fluorophenyl)trifluoroborate (228 mg, 0.96 mmol), (1,1′-bis(diphenylphosphino)ferrocene)-dichloropalladium(II) (DCM adduct) (28.1 mg, 0.03 mmol) and potassium phosphate (0.068 mL, 0.83 mmol) were suspended in DME (3 mL), water (0.75 mL) and MeOH (1.5 mL) and sealed into a microwave tube. The mixture was degassed under nitrogen and the atmosphere repaced with nitrogen. The reaction was heated to 110° C. for 3 hours in the microwave reactor and cooled to RT. The reaction mixture was evaporated to dryness and redissolved in methyl THF (50 mL), and washed sequentially with water (50 mL) and saturated brine (50 mL). The organic layer was dried over Na₂SO₄, filtered and evaporated to afford crude product. The crude product was purified by preparative HPLC (Waters XBridge Prep C18 OBD column, 5μ silica, 50 mm diameter, 150 mm length), using decreasingly polar mixtures of water (containing 0.1% formic acid) and MeCN as eluents. Fractions containing the desired compound were evaporated to dryness to afford the title compound (10.20 mg, 3.56%) as a yellow gum. (¹H NMR (400 MHz, CDCl₃, 30° C.) δ 1.51 (3H, d), 3.88-4.00 (2H, m), 4.91-5.03 (1H, m), 5.73 (1H, s), 7.06-7.15 (1H, m), 7.17-7.23 (2H, m), 7.31-7.38 (2H, m), 7.42 (1H, dd), 8.02 (1H, d), 8.33 (1H, d). m/z (ES+), (M+H)+=417

### Example 57

### (R)-4-amino-8-methyl-6-(2,2′,6′-trifluorobiphenyl-4-yl)-7,8-dihydropyrimido[5,4-f][1,4]oxazepin-5(6H)-one

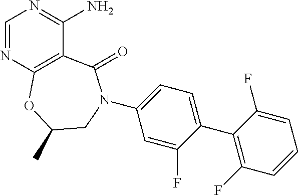

Prepared by the method of Example 56 by coupling of Intermediate 52 with potassium (2,6-difluorophenyl)trifluoroborate. ¹H NMR (400 MHz, CDCl₃, 30° C.) δ 1.51 (3H, d), 3.87-4.00 (2H, m), 4.97 (1H, td), 5.80 (1H, s), 7.02 (2H, t), 7.20 (2H, d), 7.38 (1H, tt), 7.49 (1H, dd), 8.00 (1H, s), 8.32 (1H, s). m/z (ES+), (M+H)+=401.43

### Example 58

### 4-Amino-6-(2′-chloro-2-fluorobiphenyl-4-yl)-7,8-dihydropyrimido[5,4-f][1,4]oxazepin-5(6H)-one

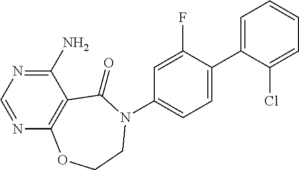

4-(4-Amino-5-oxo-7,8-dihydropyrimido[5,4-f][1,4]oxazepin-6(5H)-yl)-2-fluorophenyl trifluoromethanesulfonate (Intermediate 53; 140 mg, 0.33 mmol), 2-chlorophenylboronic acid (51.8 mg, 0.33 mmol) and tripotassium phosphate (84 mg, 0.40 mmol) were suspended in DME (3 mL), water (0.75 mL) and ethanol (1.5 mL) and degassed. (1,1′-Bis(diphenylphosphino)ferrocene)-dichloropalladium(II) (DCM adduct) (21.82 mg, 0.03 mmol) was added and the suspension was sealed into a microwave tube. The reaction was heated to 110° C. for 60 minutes in the microwave reactor and cooled to RT. The suspension was poured directly onto a SCX column and the crude reaction mixture was purified by ion exchange chromatography, using an SCX column. The column was first washed with methanol (50 mL) and the desired product was then eluted from the column using 0.35M NH₃/MeOH. Fractions containing the required product were evaporated to dryness. The crude solid was triturated with EtOAc to give a solid which was collected by filtration and dried under vacuum to give the title compound (61.0 mg, 47.8%) as a tan solid. ¹H NMR (400 MHz, DMSO, 30° C.) d 4.01-4.10 (2H, m), 4.58-4.70 (2H, m), 7.34-7.38 (1H, m), 7.40-7.53 (5H, m), 7.58-7.62 (1H, m), 7.64 (2H, s), 8.18 (1H, s). m/z (ES+) (M+H)+=385

### Example 59

### (R)-4-(4-Amino-8-methyl-5-oxo-7,8-dihydropyrimido[5,4-f][1,4]oxazepin-6(5H)-yl)-2′-chlorobiphenyl-2-carbonitrile

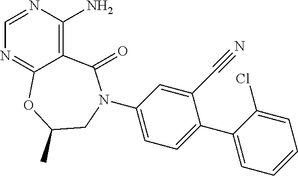

(R)-4-(4-Amino-8-methyl-5-oxo-7,8-dihydropyrimido[5,4-f][1,4]oxazepin-6(5H)-yl)-2-cyanophenyl trifluoromethanesulfonate (Intermediate 54; 58 mg, 0.13 mmol), 2-chlorophenylboronic acid (28.6 mg, 0.18 mmol), (1,1′-bis(diphenylphosphino)ferrocene)-dichloropalladium(II) (DCM adduct) (5.34 mg, 6.54 μmol) and tripotassium phosphate (33.3 mg, 0.16 mmol) were suspended in DME (3 mL), methanol (1.5 mL) and water (0.75 mL) and sealed into a microwave tube. The mixture was degassed under vacuum and the atmosphere replaced with nitrogen. The reaction was heated to 110° C. for 40 minutes in the microwave reactor and cooled to RT. The reaction mixture was evaporated to dryness and redissolved in methyl THF (100 mL), and washed sequentially with water (100 mL) and saturated brine (100 mL). A solid was filtered off and MeOH/DCM added to this and re-filtered. The filtrate was combined with the organic layer from above, filtered through a phase separating funnel and evaporated to afford crude product. The crude product was purified by flash silica chromatography, elution gradient 1 to 8% MeOH in DCM. Pure fractions were evaporated to dryness to afford the title compound (38.0 mg, 71.6%) as a white solid. ¹H NMR (400 MHz, CDCl₃) 1.53 (3H, d), 3.95 (2H, m), 4.98 (1H, m), 5.75 (1H, s), 7.35-7.46 (3H, m), 7.52-7.58 (2H, m), 7.62 (1H, dd), 7.72 (1H, d), 7.87 (1H, d), 8.34 (1H, s). m/z (ES+) (M+H)+=406.33

### Intermediate 1: 2-(4′44-Amino-5-oxo-7,8-dihydropyrimido[5,4-f][1,4]oxazepin-6(5H)-yl)-2-chlorobiphenyl-4-yl)acetic acid

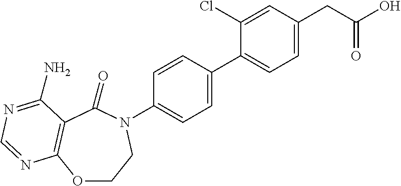

Aqueous lithium hydroxide (1M; 7.79 mL, 7.79 mmol) was added dropwise to methyl 2-(4′-(4-amino-5-oxo-7,8-dihydropyrimido[5,4-f][1,4]oxazepin-6(5H)-yl)-2-chlorobiphenyl-4-yl)acetate (Intermediate 2; 1.140 g, 2.60 mmol) in dioxane (22.5 mL) and water (7.50 mL) at 20° C. under nitrogen. The resulting solution was stirred at 45° C. for 45 minutes. The mixture was cooled to room temperature and the pH adjusted to ˜3-4 with hydrochloric acid (2M; 3.90 mL) to give a thick precipitate. The resulting mixture was evaporated to remove the organic solvent and the residual suspension was diluted with water (50 mL) and stirred vigorously for 1 hour. The resulting solid was filtered off and washed with water (3×10 mL), ether (2×15 mL) and dried under vacuum to give crude material. The crude product was triturated with MeOH (15 mL) and the resulting solid filtered off and dried under vacuum to give 2-(4′-(4-amino-5-oxo-7,8-dihydropyrimido[5,4-f][1,4]oxazepin-6(5H)-yl)-2-chlorobiphenyl-4-yl)acetic acid (0.799 g, 72.4%) as a white solid. ¹H NMR (400 MHz, DMSO) δ 3.74 (2H, s), 4.12 (2H, t), 4.70 (2H, t), 7.38-7.40 (1H, m), 7.45 (1H, d), 7.53-7.58 (5H, m), 7.69 (2H, s), 8.25 (1H, s), 12.51 (1H, s) m/z (ES+) (M+H)+=425, 427

### Intermediate 2: Methyl 2-(4′44-amino-5-oxo-7,8-dihydropyrimido[5,4-f][1,4]oxazepin-6(5H)-yl)-2-chlorobiphenyl-4-yl)acetate

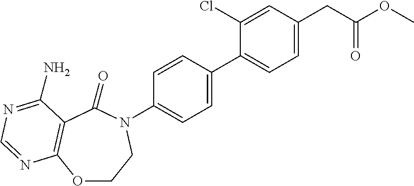

4-Amino-6-(4-bromophenyl)-7,8-dihydropyrimido[5,4-f][1,4]oxazepin-5(6H)-one (Intermediate 3; 214 mg, 0.64 mmol), methyl 2-(3-chloro-4-(4,4,5,5-tetramethyl-1,3,2-dioxaborolan-2-yl)phenyl)acetate (Intermediate 9, 198 mg, 0.64 mmol) and tripotassium phosphate (163 mg, 0.77 mmol) were suspended in DME (4 mL), methanol (2.0 mL) and water (1.0 mL) and sealed into a microwave tube. The mixture was degassed under vacuum and the atmosphere replaced with nitrogen. (1,1′-Bis(diphenylphosphino)ferrocene)-dichloropalladium(II) (DCM adduct) (26.3 mg, 0.03 mmol) was added and the reaction was heated to 110° C. for 30 minutes in the microwave reactor and cooled to RT. The reaction mixture was evaporated to dryness and redissolved in DCM (50 mL), and washed with water (20 mL). The organic layer was dried over Na₂SO₄, filtered and evaporated to afford crude product. The crude product was purified by flash silica chromatography, elution gradient 10 to 100% EtOAc in isohexane followed by 0 to 30% MeOH in EtOAc. Pure fractions were evaporated to dryness to afford methyl 2-(4′-(4-amino-5-oxo-7,8-dihydropyrimido[5,4-f][1,4]oxazepin-6(5H)-yl)-2-chlorobiphenyl-4-yl)acetate (129 mg, 46.0%) as a white solid. ¹H NMR (400 MHz, DMSO) δ 3.65 (3H, s), 3.78 (2H, s), 4.05 (2H, t), 4.63 (2H, t), 7.32-7.35 (1H, m), 7.36-7.39 (2H, m), 7.41-7.44 (1H, m), 7.49-7.52 (3H, m), 7.62 (2H, s), 8.18 (1H, s). m/z (ES+) (M+H)+=439, 441

### Intermediate 3: 4-Amino-6-(4-bromophenyl)-7,8-dihydropyrimido[5,4-f][1,4]oxazepin-5(6H)-one

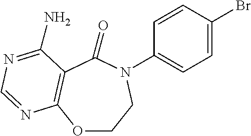

6-(4-Bromophenyl)-4-chloro-7,8-dihydropyrimido[5,4-f][1,4]oxazepin-5(6H)-one (Intermediate 4; 4.77 g, 13.45 mmol) was added in one portion to a stirring solution of 0.5M ammonia in 1,4-dioxane (155 mL) at 20° C. The resulting solution was stirred at 45° C. for 8 hours followed by the addition of further 0.5M ammonia in 1,4-dioxane (60 mL) and stirring at 20° C. overnight. Analysis showed the reaction to be ˜80% complete, so further 0.5M ammonia in 1,4-dioxane (60 mL) was added and the mixture stirred at 45° C. for a further 6 hours. The reaction mixture was evaporated to dryness and the residue partitioned between water (500 mL) and EtOAc (500 mL). A white precipitate was filtered from the biphasic mixture and washed with water (100 mL) and EtOAc (200 mL), and dried under vacuum to give 4-amino-6-(4-bromophenyl)-7,8-dihydropyrimido[5,4-f][1,4]oxazepin-5(6H)-one (3.41 g, 76%) as a white solid.

¹H NMR (400 MHz, DMSO) δ 4.05 (2H, t), 4.66 (2H, t), 7.43 (2H, d), 7.66-7.70 (4H, m), 8.23 (1H, s). m/z (ES+) (M+H)+=335, 337

### Intermediate 4: 6-(4-Bromophenyl)-4-chloro-7,8-dihydropyrimido[5,4-f][1,4]oxazepin-5(6H)-one

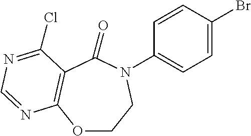

Triethylamine (9.23 mL, 66.39 mmol) was added in one portion to N-(4-bromophenyl)-4,6-dichloro-N-(2-hydroxyethyl)pyrimidine-5-carboxamide (Intermediate 5; 6.832 g, 17.47 mmol) in acetonitrile (78 mL) at 20° C. under nitrogen. The resulting solution was stirred at 80° C. for 6 hours. The reaction mixture was evaporated to dryness and redissolved in EtOAc (500 mL), and washed with water (3×100 mL) and saturated brine (100 mL). The organic layer was dried over Na₂SO₄, filtered and evaporated to afford crude product. The crude solid was triturated with MeOH (50 mL) to give a solid which was collected by filtration, washed with MeOH (25 mL) and ether (50 mL) and dried under vacuum to give 6-(4-bromophenyl)-4-chloro-7,8-dihydropyrimido[5,4-f][1,4]oxazepin-5(6H)-one (4.73 g, 76%) as a white solid. ¹H NMR (400 MHz, DMSO) δ 4.23 (2H, t), 4.79 (2H, t), 7.49 (2H, d), 7.73 (2H, d), 8.89 (1H, s). m/z (ES+) (M+H)+=354, 356

### Intermediate 5: N-(4-bromophenyl)-4,6-dichloro-N-(2-hydroxyethyl)pyrimidine-5-carboxamide

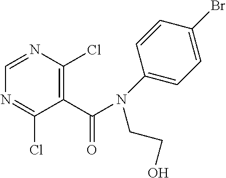

N-(4-bromophenyl)-N-(2-(tert-butyldimethylsilyloxy)ethyl)-4,6-dichloropyrimidine-5-carboxamide (Intermediate 6; 6.06 g, 11.99 mmol) as a solution in methanol (8.0 mL) was added in one portion to a solution of conc hydrochloric acid (1.546 mL) in methanol (50 mL) at 20° C. under nitrogen. The resulting solution was stirred at 20° C. for 30 minutes. The reaction mixture was evaporated to dryness and redissolved in EtOAc (150 mL), and washed sequentially with saturated NaHCO₃ (100 mL) and saturated brine (100 mL). The organic layer was dried over Na₂SO₄, filtered and evaporated to afford crude product. The crude product was purified by flash silica chromatography, elution gradient 10 to 90% EtOAc in isohexane. Pure fractions were evaporated to dryness to afford N-(4-bromophenyl)-4,6-dichloro-N-(2-hydroxyethyl)pyrimidine-5-carboxamide (4.88 g, 104%) as a colourless oil. ¹H NMR (400 MHz, DMSO) δ 3.57-3.62 (2H, m), 3.90 (2H, t), 4.84 (1H, t), 7.37 (2H, d), 7.55 (2H, d), 8.83 (1H, s). m/z=mass ion not seen

### Intermediate 6: N-(4-bromophenyl)-N-(2-(tert-butyldimethylsilyloxy)ethyl)-4,6-dichloropyrimidine-5-carboxamide

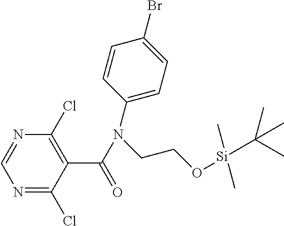

4,6-Dichloropyrimidine-5-carbonyl chloride (WO 2009016462) (2.53 g, 11.95 mmol) as a solution in THF (15 mL) was added dropwise to 4-bromo-N-(2-(tert-butyldimethylsilyloxy)ethyl)aniline (Intermediate 7; 3.76 g, 11.38 mmol) and triethylamine (1.709 mL, 12.29 mmol) in THF (30.0 mL) at 0° C. over a period of 1 minute under nitrogen. The resulting solution was stirred at 0° C. for 2 hours. The reaction mixture was evaporated to dryness and redissolved in EtOAc (150 mL), and washed sequentially with water (3×75 mL) and saturated brine (75 mL). The organic layer was dried over Na₂SO₄, filtered and evaporated to afford crude product as an orange oil. The crude product was taken on without further characterisation

### Intermediate 7: 4-Bromo-N-(2-(tert-butyldimethylsilyloxy)ethyl)aniline

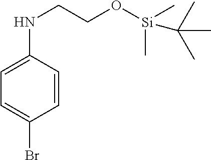

(2-Bromoethoxy)(tert-butyl)dimethylsilane (8.61 mL, 40.13 mmol) was added dropwise to 4-bromoaniline (6.28 g, 36.48 mmol) and sodium carbonate (7.73 g, 72.96 mmol) in DMF (60 mL) at 20° C. over a period of 3 minutes under nitrogen. The resulting suspension was stirred at 60° C. for 3 days. The reaction was concentrated to ˜20 mL volume under vacuum and added dropwise to cold water (500 mL). The aqueous was extracted with EtOAc (2×200 mL), and the combined organics washed with brine (2×100 mL), dried (MgSO₄) and evaporated under vacuum to give crude material. The crude product was purified by flash silica chromatography, elution gradient 5 to 100% DCM in isohexane. Pure fractions were evaporated to dryness to afford 4-bromo-N-(2-(tert-butyldimethylsilyloxy)ethyl)aniline (6.23 g, 51.7%) as a orange oil.

¹H NMR (400 MHz, DMSO) δ 0.00 (6H, s), 0.83 (9H, s), 3.07-3.12 (2H, m), 3.66 (2H, t), 5.69 (1H, t), 6.52 (2H, d), 7.15 (2H, d). m/z (ES+) (M+H)+=330, 332

### Intermediate 8: 4-Amino-6-(4-(4,4,5,5-tetramethyl-1,3,2-dioxaborolan-2-yl)phenyl)-7,8-dihydropyrimido[5,4-f][1,4]oxazepin-5(6H)-one

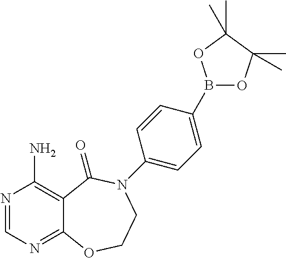

4-Amino-6-(4-bromophenyl)-7,8-dihydropyrimido[5,4-f][1,4]oxazepin-5(6H)-one (Intermediate 4; 470 mg, 1.40 mmol), potassium acetate (385 mg, 3.93 mmol) and bis(pinacolato)diboron (445 mg, 1.75 mmol) were suspended in dioxane (12 mL) and sealed into a microwave tube. The tube was degassed under vacuum and the atmosphere replaced with nitrogen. The mixture was treated with PdCl₂(dppf)-CH₂Cl₂ adduct (68.7 mg, 0.08 mmol) and the reaction was heated to 130° C. for 40 minutes in the microwave reactor and cooled to RT. The reaction mixture was evaporated to dryness and redissolved in DCM (50 mL) and washed with water (50 mL). The organic layer was dried over Na₂SO₄, filtered and evaporated to afford crude product. The crude product was purified by flash silica chromatography, elution gradient 50 to 100% EtOAc in isohexane followed by 0 to 20% MeOH in EtOAc. Pure fractions were evaporated to dryness to afford 4-amino-6-(4-(4,4,5,5-tetramethyl-1,3,2-dioxaborolan-2-yl)phenyl)-7,8-dihydropyrimido[5,4-f][1,4]oxazepin-5(6H)-one (314 mg, 58.6%) as a white solid.

### Intermediate 9: Methyl 2-(3-chloro-4-(4,4,5,5-tetramethyl-1,3,2-dioxaborolan-2-yl)phenyl)acetate

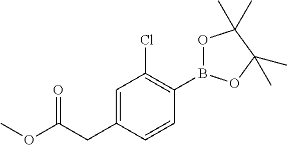

To a degassed solution of methyl 2-(3-chloro-4-(trifluoromethylsulfonyloxy)phenyl)acetate (Intermediate 1-2 from WO2010/146395; 6.56 g, 19.72 mmol) in dioxane (150 mL) was added potassium acetate (6.00 g, 61.13 mmol), bis(pinacolato)diboron (7.51 g, 29.58 mmol), 1,1′-bis(diphenylphosphino)ferrocene (0.663 g, 1.18 mmol) and PdCl₂(dppf)-CH₂Cl₂ adduct (0.966 g, 1.18 mmol). The suspension was degassed and then heated, under nitrogen, to 100° C. overnight. The reaction was incomplete and further PdCl₂(dppf)-CH₂Cl₂ adduct (0.966 g, 1.18 mmol) was added and the mixture was stirred at 100° C. for a further 4 hours. The reaction mixture was allowed to cool, concentrated and diluted with EtOAc (300 mL), and washed with saturated brine (300 mL). The organic layer was dried over MgSO₄, filtered and evaporated to afford crude product which was filtered through a pad of silica (1″×3″), washing through with EtOAc. The crude product was purified by flash silica chromatography, elution gradient 0 to 20% EtOAc in isohexane. Pure fractions were evaporated to dryness to afford methyl 2-(3-chloro-4-(4,4,5,5-tetramethyl-1,3,2-dioxaborolan-2-yl)phenyl)acetate (6.11 g, 100%) as a colourless oil which solidified on standing. ¹H NMR (400.132 MHz, CDCl₃) δ 1.36 (12H, s), 3.59 (2H, s), 3.68 (3H, s), 7.14-7.16 (1H, m), 7.28 (1H, s), 7.65 (1H, d).

### Intermediate 10: 2-(2-Chloro-4-(methylsulfonylmethyl)phenyl)-4,4,5,5-tetramethyl-1,3,2-dioxaborolane

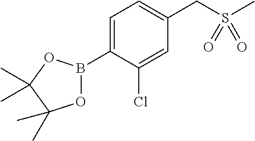

1-Bromo-2-chloro-4-(methylsulfonylmethyl)benzene (Intermediate 11; 140 mg, 0.49 mmol), bis(pinacolato)diboron (188 mg, 0.74 mmol), potassium acetate (150 mg, 1.53 mmol) and (1,1′-bis(diphenylphosphino)ferrocene)-dichloropalladium(II) (DCM adduct) (24.19 mg, 0.03 mmol) were suspended in dioxane (5 mL) and sealed into a microwave tube. The suspension was degassed by evacuation and inlet of nitrogen. The reaction was heated to 120° C. for 45 minutes in the microwave reactor and cooled to RT. The solvent was evaporated and the residue suspended in EtOAc and water. This was filtered through celite and the residue evaporated. The crude product was purified by flash silica chromatography, elution gradient 5 to 70% EtOAc in isohexane. Pure fractions were evaporated to dryness to afford 2-(2-chloro-4-(methylsulfonylmethyl)phenyl)-4,4,5,5-tetramethyl-1,3,2-dioxaborolane (80 mg, 49.0%) as a colourless oil.

¹H NMR (400 MHz, CDCl₃) δ 1.37 (12H, s), 2.74 (3H, s), 4.20 (2H, s), 7.28-7.32 (1H, m), 7.39-7.43 (1H, m), 7.73 (1H, d).

### Intermediate 11: 1-Bromo-2-chloro-4-(methylsulfonylmethyl)benzene

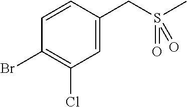

3-Chloroperoxybenzoic acid (2.59 g, 11.54 mmol) was added to a stirred solution of (4-bromo-3-chlorobenzyl)(methyl)sulfane (Intermediate 12; 1.32 g, 5.25 mmol) in DCM (30 mL) at 0° C. The solution was allowed stir for 30 mins. The reaction was quenched with sodium thiosulphate (5 mL). The organic layer was separated and washed with sat. NaHCO₃ and concentrated in vacuo to give crude product. The crude product was purified by flash silica chromatography, elution gradient 0 to 100% EtOAC in heptane. Pure fractions were evaporated to dryness to afford 1-bromo-2-chloro-4-(methylsulfonylmethyl)benzene (0.140 g, 9.41%) as a white solid. ¹H NMR (400 MHz, CDCl₃) δ 2.83 (3H, s), 4.19 (2H, s), 7.20 (1H, dd), 7.52 (1H, d), 7.68 (1H, d)

### Intermediate 12: (4-Bromo-3-chlorobenzyl)(methyl)sulfane

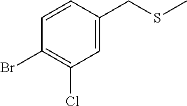

Sodium methanethiolate (6.68 mL, 22.03 mmol) was added to a stirred solution of 4-bromo-3-chlorobenzyl methanesulfonate (Intermediate 13; 3.3 g, 11.02 mmol) in THF (50 mL) at room temperature. The solution was allowed to warm to ambient temperature and stir for 30 mins. The reaction mixture was diluted with EtOAc (100 mL) and water (50 mL). The organic layer was separated, washed with sat NaHCO₃ (50 mL) then dried over anh. MgSO₄ and evaporated to give (4-bromo-3-chlorobenzyl)(methyl)sulfane (2.65 g, 96%) as a colourless oil which solidified on standing. ¹H NMR (400 MHz, CDCl₃) δ 1.92 (3H, s), 3.52 (2H, s), 7.00 (1H, dd), 7.34 (1H, d), 7.44-7.50 (1H, m).

### Intermediate 13: 4-Bromo-3-chlorobenzyl methanesulfonate

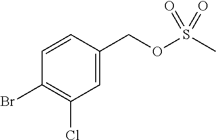

Methanesulfonyl chloride (1.660 mL, 21.36 mmol) was added dropwise to (4-bromo-3-chlorophenyl)methanol (4.3 g, 19.41 mmol) and triethylamine (3.38 mL, 24.27 mmol) in DCM (50 mL) cooled to 0° C. under nitrogen. The resulting solution was stirred at 0° C. for 1 hour. The reaction mixture was then washed with water (25 mL) and saturated NaHCO₃ (25 mL). The organic layer was dried over MgSO₄, filtered and evaporated to afford 4-bromo-3-chlorobenzyl methanesulfonate (5.36 g, 92%) as an oil. ¹H NMR (400 MHz, CDCl₃) δ 2.93 (3H, s), 5.09 (2H, s), 7.07-7.14 (1H, m), 7.44 (1H, t), 7.55-7.65 (1H, m).

### Intermediate 14: N-(3-chloro-4-(4,4,5,5-tetramethyl-1,3,2-dioxaborolan-2-yl)benzyl)-N-methylmethanesulfonamide

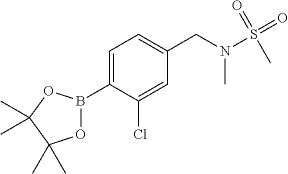

(1,1′-Bis(diphenylphosphino)ferrocene)-dichloropalladium(II) (DCM adduct) (0.042 g, 0.05 mmol) was added to N-(4-bromo-3-chlorobenzyl)-N-methylmethanesulfonamide (Intermediate 15; 0.27 g, 0.86 mmol), bis(pinacolato)diboron (0.329 g, 1.30 mmol) and potassium acetate (0.263 g, 2.68 mmol) in dioxane (15 mL). The suspension was degassed by evacuation and inlet of nitrogen and then heated, under nitrogen, to reflux overnight. The reaction mixture was allowed to cool, evaporated and the residue suspended in EtOAc and water. This was filtered through celite and the residue evaporated. The crude product was purified by flash silica chromatography, elution gradient 1 to 10% MeOH in DCM. Pure fractions were evaporated to dryness to afford N-(3-chloro-4-(4,4,5,5-tetramethyl-1,3,2-dioxaborolan-2-yl)benzyl)-N-methylmethanesulfonamide (0.166 g, 53.4%) as a colourless oil. ¹H NMR (400 MHz, CDCl₃) δ 1.37 (12H, s), 2.75 (3H, s), 2.81 2.86 (3H, m), 4.29 (2H, d), 7.21 7.25 (1H, m), 7.35 (1H, t), 7.68 (1H, d). m/z (ES+) (M+H)+=360

### Intermediate 15: N-(4-bromo-3-chlorobenzyl)-N-methylmethanesulfonamide

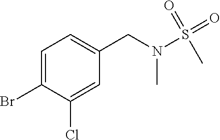

Pyridine (0.414 mL, 5.12 mmol) was added to 1-(4-bromo-3-chlorophenyl)-N-methylmethanamine (Intermediate 16; 0.3 g, 1.28 mmol) and methanesulfonyl chloride (0.149 mL, 1.92 mmol) in DCM (5 mL) at 20° C. Stirred at 20° C. for 20 hours. The reaction mixture was diluted with DCM (30 mL), washed with 1M citric acid (20 mL) and saturated brine (20 mL) and the organic layer filtered through a phase separation tube to give crude product. The crude product was purified by flash silica chromatography, elution gradient 5 to 40% EtOAc in isohexane. Pure fractions were evaporated to dryness to afford N-(4-bromo-3-chlorobenzyl)-N-methylmethanesulfonamide (0.270 g, 67.5%) as a colourless oil. ¹H NMR (400 MHz, CDCl₃) δ 2.78 (3H, s), 2.87 (3H, s), 4.24 (2H, s), 7.13 (1H, dd), 7.45 (1H, d), 7.59 7.62 (1H, m).

### Intermediate 16: 1-(4-Bromo-3-chlorophenyl)-N-methylmethanamine

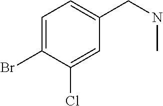

Sodium tetrahydroborate (0.566 g, 14.97 mmol) was added in one portion to (Z)—N-(4-bromo-3-chlorobenzylidene)methanamine (Intermediate 17; 3.48 g, 14.97 mmol) in MeOH (150 mL) at 20° C. under nitrogen. The resulting mixture was stirred at RT for 2 hours. The reaction mixture was concentrated to approximately half its volume and then the reaction mixture was quenched with saturated NH₄Cl (25 mL), extracted with EtOAc (3×75 mL). (Water was added to dissolve formed precipitate). The combined organic layers was dried over Na₂SO₄, filtered and evaporated. The crude product was purified by ion exchange chromatography, using an SCX column. The desired product was eluted from the column using 7M NH₃/MeOH and pure fractions were evaporated to dryness to afford 1-(4-bromo-3-chlorophenyl)-N-methylmethanamine (2.43 g, 69.2%) as a golden oil.

¹H NMR (400 MHz, CDCl₃) δ 1.37 (1H, s), 2.43 (3H, s), 3.69 (2H, s), 7.08 (1H, dd), 7.44 (1H, d), 7.53 7.56 (1H, m). m/z (ES+) (M+H)+=234, 236

### Intermediate 17: (Z)—N-(4-bromo-3-chlorobenzylidene)methanamine

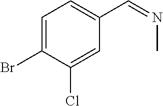

Sodium triacetoxyborohydride (4.81 g, 22.69 mmol) was added in one portion to 4-bromo-3-chlorobenzaldehyde (3.32 g, 15.13 mmol), acetic acid (0.866 mL, 15.13 mmol) and methylamine (2M in THF) (37.8 mL, 75.64 mmol) in THF (80 mL) at 20° C. under nitrogen. The resulting mixture was stirred at R.T. for 4 hours. The reaction mixture was concentrated ˜half volume and then quenched with saturated NaHCO₃ (50 mL), extracted with EtOAc (100 mL). The combined organics were washed with brine (50 mL), dried over Na₂SO₄, filtered and evaporated to afford (Z)—N-(4-bromo-3-chlorobenzylidene)methanamine (3.48 g, 99%) as a yellow oil. The crude product containing ˜7% of the corresponding amine reduction product and ˜10% of the starting aldehyde was used without further purification.

### Intermediate 18: N-(3-chloro-4-(4,4,5,5-tetramethyl-1,3,2-dioxaborolan-2-yl)benzyl)-N-methylacetamide

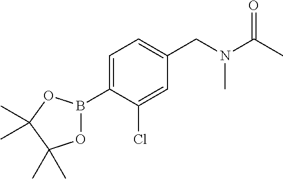

(1,1′-Bis(diphenylphosphino)ferrocene)-dichloropalladium(II) (DCM adduct) (0.034 g, 0.04 mmol) was added to a stirred, degassed mixture of N-(4-bromo-3-chlorobenzyl)-N-methylacetamide (Intermediate 19; 0.19 g, 0.69 mmol), potassium acetate (0.209 g, 2.13 mmol) and bis(pinacolato)diboron (0.262 g, 1.03 mmol) in dioxane (8 mL). The suspension was degassed by evacuation and inlet of nitrogen and then heated, under nitrogen, to reflux overnight. The reaction mixture was allowed to cool, evaporated and the residue suspended in EtOAc and water. This was filtered through celite and the residue evaporated. The crude product was purified by flash silica chromatography, elution gradient 1 to 8% MeOH in DCM. Pure fractions were evaporated to dryness to afford N-(3-chloro-4-(4,4,5,5-tetramethyl-1,3,2-dioxaborolan-2-yl)benzyl)-N-methylacetamide (0.220 g, 99%) as a brown oil.

¹H NMR (400 MHz, CDCl₃) δ 1.25 1.29 (12H, m), 2.08 2.18 (3H, m), 2.93 (3H, dd), 4.47 4.58 (2H, m), 6.98 7.23 (2H, m), 7.66 (1H, dd). m/z (ES+) (M+H)+=324

### Intermediate 19: N-(3-chloro-4-(4,4,5,5-tetramethyl-1,3,2-dioxaborolan-2-yl)benzyl)-N-methylacetamide

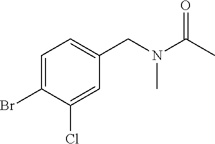

O-(7-Azabenzotriazol-1-yl)-N,N,N′,N′-tetramethyluronium hexafluorophosphate (0.973 g, 2.56 mmol) was added portionwise to 1-(4-bromo-3-chlorophenyl)-N-methylmethanamine (Intermediate 16; 0.4 g, 1.71 mmol), acetic acid (0.107 mL, 1.88 mmol) and DIPEA (1.188 mL, 6.82 mmol) in DMF (10 mL) at RT under nitrogen. The resulting solution was stirred at RT overnight. The reaction mixture was diluted with EtOAc (50 mL), and washed sequentially with saturated NaHCO₃ (25 mL), saturated brine (25 mL), and water (25 mL). The organic layer was dried over MgSO₄, filtered and evaporated to afford crude product. The crude product was purified by flash silica chromatography, elution gradient 0 to 8% MeOH in DCM. Fractions were evaporated to dryness to afford N-(4-bromo-3-chlorobenzyl)-N-methylacetamide (0.190 g, 40.3%) as a brown gum.

¹H NMR (400 MHz, CDCl₃) δ 2.15 (3H, d), 2.94 (3H, d), 4.49 (2H, d), 6.97 (1H, ddd), 7.30 (1H, dd), 7.52-7.64 (1H, m). m/z (ES+) (M+H)+=276, 278

### Intermediate 20: N-(3-chloro-4-(4,4,5,5-tetramethyl-1,3,2-dioxaborolan-2-yl)benzyl)methanesulfonamide

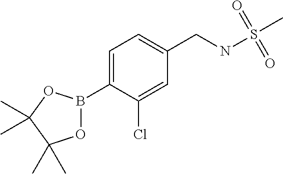

N-(4-Bromo-3-chlorobenzyl)methanesulfonamide (Intermediate 21; 0.245 g, 0.82 mmol), bis(pinacolato)diboron (0.313 g, 1.23 mmol), potassium acetate (0.250 g, 2.54 mmol) and (1,1′-bis(diphenylphosphino)ferrocene)-dichloropalladium(II) (DCM adduct) (0.040 g, 0.05 mmol) were suspended in dioxane (10 mL) and sealed into a microwave tube. The suspension was degassed by evacuation and inlet of nitrogen. The reaction was heated to 120° C. for 45 minutes in the microwave reactor and cooled to RT. The reaction mixture was concentrated and diluted with EtOAc (25 mL) and washed with water (25 mL). The organic layer was dried by passing through a phase separating cartridge and evaporated to afford crude product. The crude product was purified by flash silica chromatography, elution gradient 0 to 4% MeOH in DCM. Pure fractions were evaporated to dryness to afford crude N-(3-chloro-4-(4,4,5,5-tetramethyl-1,3,2-dioxaborolan-2-yl)benzyl)methanesulfonamide (0.252 g, 89%) which was used without further purification. ¹H NMR (400 MHz, CDCl₃) δ 1.37 (12H, s), 2.88 (3H, s), 4.31 (3H, dd), 4.63 (1H, s), 7.22 (1H, dd), 7.34 (1H, d), 7.69 (1H, d).

### Intermediate 21: N-(4-bromo-3-chlorobenzyl)methanesulfonamide

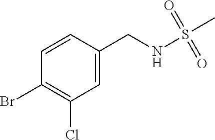

Methanesulfonyl chloride (0.097 mL, 1.25 mmol) was added portionwise to (4-bromo-3-chlorophenyl)methanamine (250 mg, 1.13 mmol) and pyridine (0.275 mL, 3.40 mmol) in DCM (5 mL) at RT under nitrogen. The resulting solution was stirred at RT for overnight. The reaction mixture was concentrated and diluted with EtOAc (25 mL) and washed with water (2×25 mL). The organic layer was dried by passing through a phase separating cartridge and evaporated to afford desired product N-(4-bromo-3-chlorobenzyl)methanesulfonamide (350 mg, 103%). ¹H NMR (400 MHz, CDCl₃) δ 2.93 (3H, s), 4.27 (2H, d), 4.68 (1H, s), 7.13 (1H, dd), 7.46 (1H, d), 7.59 7.63 (1H, m). m/z (ES−) (M−H)−=298

### Intermediate 22: N-(3-chloro-4-(4,4,5,5-tetramethyl-1,3,2-dioxaborolan-2-yl)benzyl)acetamide

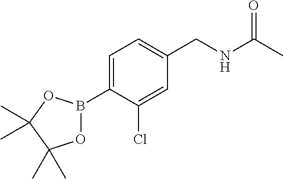

N-(4-Bromo-3-chlorobenzyl)acetamide (Intermediate 23; 0.29 g, 1.10 mmol), bis(pinacolato)diboron (0.421 g, 1.66 mmol), potassium acetate (0.336 g, 3.42 mmol) and (1,1′-bis(diphenylphosphino)ferrocene)-dichloropalladium(II) (DCM adduct) (0.054 g, 0.07 mmol) were suspended in dioxane (10 mL) and sealed into a microwave tube. The suspension was degassed by evacuation and inlet of nitrogen. The reaction was heated to 120° C. for 45 minutes in the microwave reactor and cooled to RT. The reaction mixture was concentrated and diluted with EtOAc (25 mL) and washed with water (25 mL). The organic layer was dried by passing through a phase separating cartridge and evaporated to afford crude product. The crude product was purified by flash Silica chromatography, elution gradient 0 to 4% MeOH in DCM. Pure fractions were evaporated to dryness to afford crude N-(3-chloro-4-(4,4,5,5-tetramethyl-1,3,2-dioxaborolan-2-yl)benzyl)acetamide (0.270 g, 79%) as a colourless solid which was used without further purification.

¹H NMR (400 MHz, CDCl₃) δ 1.36 (12H, s), 2.04 (3H, s), 4.41 (2H, d), 5.71 (1H, s), 7.14 (1H, dd), 7.65 (1H, d). m/z (ES+) (M+H)+=310

### Intermediate 23: N-(4-bromo-3-chlorobenzyl)acetamide

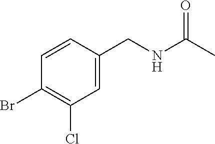

Acetyl chloride (0.081 mL, 1.13 mmol) was added portionwise to (4-bromo-3-chlorophenyl)methanamine (250 mg, 1.13 mmol) and DIPEA (0.592 mL, 3.40 mmol) in DCM (5 mL) at RT under nitrogen. The resulting solution was stirred at RT for overnight. The reaction mixture was concentrated and diluted with EtOAc (25 mL) and washed with water (2×25 mL). The organic layer was dried by passing through a phase separating cartridge and evaporated to afford desired product N-(4-bromo-3-chlorobenzyl)acetamide (300 mg, 101%). ¹H NMR (400 MHz, CDCl₃) δ 2.04 (3H, s), 4.37 (2H, d), 5.79 (1H, s), 7.04 (1H, dd), 7.37 (1H, d), 7.54 7.58 (1H, m). m/z (ES+) (M+H)+=262; 264; 266

### Intermediate 24: 1-Bromo-2-chloro-4-(difluoromethyl)benzene

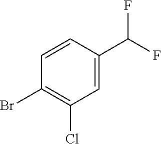

Ethanol (0.053 mL, 0.91 mmol) was added to 4-bromo-3-chlorobenzaldehyde (1 g, 4.56 mmol) and Deoxo-Fluor® (50% in THF) (3.36 mL, 7.75 mmol) in DCM (20 mL) at 22° C. The resulting solution was stirred at 22° C. for 20 hours. The reaction mixture was quenched with saturated NaHCO₃ (100 mL), extracted with DCM (2×150 mL), the organic layer was dried over Na₂SO₄, filtered and evaporated to afford a yellow liquid. The crude product was purified by flash alumina chromatography, elution gradient 1 to 5% EtOAc in isohexane. Pure fractions were evaporated to dryness to afford the title compound (0.750 g, 68.2%) as a colourless liquid, containing 4.5% starting aldehyde by NMR. The product was used in subsequent reactions without further purification

¹H NMR (400 MHz, CDCl₃) 6.59 (1H, td), 7.23-7.29 (1H, m), 7.60 (1H, s), 7.72 (1H, d).

### Intermediate 25: 2-Chloro-3-cyanophenyl trifluoromethanesulfonate

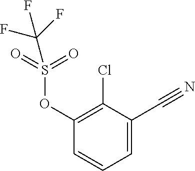

Potassium carbonate (1.350 g, 9.77 mmol) was added to 2-chloro-3-hydroxybenzonitrile (Intermediate 25a; 0.5 g, 3.26 mmol) and 1,1,1-trifluoro-N-phenyl-N-(trifluoromethylsulfonyl)methanesulfonamide (1.163 g, 3.26 mmol) in THF (40 mL) at 20° C. The resulting suspension was stirred at 20° C. for 50 hours. The reaction mixture was evaporated to dryness and redissolved in EtOAc (25 mL), and washed sequentially with water (25 mL) and saturated brine (25 mL). The organic layer was dried over Na₂SO₄, filtered and evaporated to afford crude product. The crude product was purified by flash silica chromatography, elution gradient 5 to 20% EtOAc in isohexane. Pure fractions were evaporated to dryness to afford 2-chloro-3-cyanophenyl trifluoromethanesulfonate (0.696 g, 74.8%) as a colourless liquid. ¹H NMR (400 MHz, CDCl₃) 7.47 7.53 (1H, m), 7.62 (1H, dd), 7.73 (1H, dd). m/z (ES−) (M−H)−=284

### Intermediate 25a: 2-chloro-3-hydroxybenzonitrile

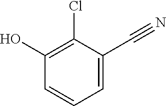

Iodocyclohexane (3.86 mL, 29.83 mmol) was added to 2-chloro-3-methoxybenzonitrile (1 g, 5.97 mmol) in DMF (10 mL) at 20° C. under nitrogen. The resulting solution was stirred at 155° C. for 7 hours. The reaction was cooled and poured into water. Extracted into ethyl acetate (2×50 mL) and the combined organics washed with brine (50 mL), dried over Na₂SO₄, filtered and evaporated to give crude product. The crude solid was triturated with DCM to give a solid which was collected by filtration and dried under vacuum to give 2-chloro-3-hydroxybenzonitrile (0.510 g, 55.7%) as a white solid. No further material obtained after columning the filtrate. ¹H NMR (400 MHz, DMSO) 7.25 7.39 (3H, m), 11.00 (1H, s). m/z (ES−) (M−H)−=152

### Intermediate 26: 2-Chloro-6-cyanophenyl trifluoromethanesulfonate

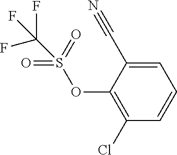

Trifluoromethanesulfonic anhydride (2.157 mL, 12.82 mmol) was added dropwise to 3-chloro-2-hydroxybenzamide (1 g, 5.83 mmol) and triethylamine (2.68 mL, 19.23 mmol) in DCM (50 mL) at 0° C. over a period of 10 minutes under nitrogen. The resulting solution was stirred at 20° C. for 90 minutes. The reaction mixture was diluted with DCM (50 mL), and washed sequentially with water (100 mL), and saturated brine (100 mL). The organic layer was dried over Na₂SO₄, filtered and evaporated to afford crude product. The crude product was purified by flash silica chromatography, elution gradient 5 to 30% EtOAc in isohexane. Pure fractions were evaporated to dryness to afford 2-chloro-6-cyanophenyl trifluoromethanesulfonate (1.100 g, 66.1%) as a yellow liquid. ¹H NMR (400 MHz, CDCl₃) 7.47 (1H, t), 7.69 (1H, dd), 7.80 (1H, dd). m/z (ES−) (M−H)−=284

### Intermediate 27: (S)-4-Amino-6-(4′-((3-(tert-butyldimethylsilyloxy)-2-oxopyrrolidin-1-yl)methyl)-2′-chlorobiphenyl-4-yl)-7,8-dihydropyrimido[5,4-f][1,4]oxazepin-5(6H)-one

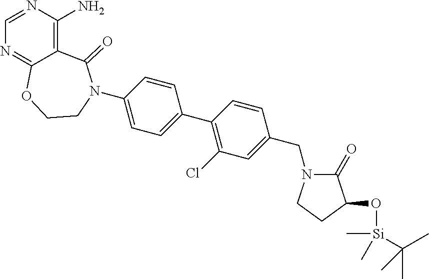

4-Amino-6-(4-bromophenyl)-7,8-dihydropyrimido[5,4-f][1,4]oxazepin-5(6H)-one (Intermediate 28; 0.1 g, 0.30 mmol), (S)-3-(tert-butyldimethylsilyloxy)-1-(3-chloro-4-(4,4,5,5-tetramethyl-1,3,2-dioxaborolan-2-yl)benzyl)pyrrolidin-2-one (0.139 g, 0.30 mmol), (1,1′-bis(diphenylphosphino)ferrocene)-dichloropalladium(II) (DCM adduct) (0.012 g, 0.01 mmol) and tripotassium phosphate (0.076 g, 0.36 mmol) were suspended in DME (3 mL), methanol (1.5 mL) and water (0.75 mL) and sealed into a microwave tube. The mixture was degassed under vacuum and the atmosphere replaced with nitrogen. The reaction was heated to 110° C. for 40 minutes in the microwave reactor and cooled to RT. The reaction mixture was evaporated to dryness and redissolved in methyl THF (100 mL), and washed sequentially with water (100 mL) and saturated brine (100 mL). A solid was filtered off and MeOH/DCM added to this and re-filtered. The filtrate was combined with the organic layer from above, filtered through a phase separating funnel and evaporated to afford crude product. The crude product was purified by flash silica chromatography, elution gradient 1 to 10% MeOH in DCM. Pure fractions were evaporated to dryness to afford the crude title compound (0.070 g, 39.5%) as a white solid which was used without further purification.

¹H NMR (400 MHz, CDCl₃) −0.04-−0.01 (3H, m), −0.01-0.04 (3H, m), 0.71-0.76 (9H, m), 1.70-1.79 (1H, m), 2.13 (1H, dtd), 2.99 (1H, dt), 3.07-3.20 (1H, m), 3.85-3.95 (2H, m), 4.13-4.21 (1H, m), 4.22-4.34 (2H, m), 4.52 (2H, ddd), 5.51 (1H, s), 6.90-7.21 (5H, m), 7.24-7.41 (2H, m), 7.93 (1H, d), 8.08-8.15 (1H, m). m/z (ES+) M+=594

### Intermediate 28: (S)-3-(tert-butyldimethylsilyloxy)-1-(3-chloro-4-(4,4,5,5-tetramethyl-1,3,2-dioxaborolan-2-yl)benzyl)pyrrolidin-2-one

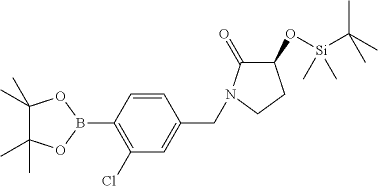 [0612] (S)-1-(4-Bromo-3-chlorobenzyl)-3-(tert-butyldimethylsilyloxy)pyrrolidin-2-one (Intermediate 29; 0.8 g, 1.91 mmol), potassium acetate (0.562 g, 5.73 mmol), (1,1′-bis(diphenylphosphino)ferrocene)-dichloropalladium(II) (DCM adduct) (0.109 g, 0.13 mmol) and bis(pinacolato)diboron (0.582 g, 2.29 mmol) were suspended in a microwave tube. The suspension was degassed. The reaction was heated to 130° C. for 2 hours in the microwave reactor and cooled to RT and for a further 3 hrs. The reaction mixture was evaporated and the residue taken up in methanol and filtered. The filtrate was evaporated and partitioned between water (80 mL) and methyl THF (100 mL). The organic phase was separated and evaporated. The crude product was purified by flash silica chromatography, elution gradient 5 to 30% EtOAc in isohexane. The crude product was re-purified by flash silica chromatography, elution gradient 100% DCM followed by 1 to 10% MeOH in DCM to elute off the product. Pure fractions were evaporated to dryness to afford title product (0.130 g, 14.61%) as a yellow oil. ¹H NMR (400 MHz, CDCl₃) −0.03 (3H, s), −0.01-0.04 (3H, m), 0.72-0.75 (9H, m), 1.17 (12H, s), 1.62-1.75 (1H, m), 2.02-2.15 (1H, m), 2.89 (1H, ddt), 3.02 (1H, ddd), 4.16 (1H, dd), 4.21 (2H, dd), 6.92 (1H, dt), 7.02 (1H, t), 7.45 (1H, d). m/z (ES+) (M+H)+=466

### Intermediate 29: (S)-1-(4-bromo-3-chlorobenzyl)-3-(tert-butyldimethylsilyloxy)pyrrolidin-2-one

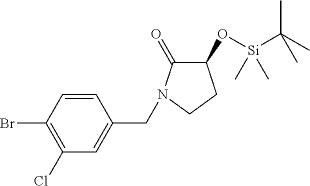

Sodium hydride (0.176 g, 4.41 mmol) was added to (S)-3-(tert-butyldimethylsilyloxy)pyrrolidin-2-one (Intermediate 30; 0.863 g, 4.01 mmol) in DMF (10 mL) at 0° C. under nitrogen. The resulting solution was stirred at 20° C. for 30 minutes and recooled to 0° C. 4-bromo-3-chlorobenzyl methanesulfonate (Intermediate 13; 1.2 g, 4.01 mmol) was added and the reaction stirred at 20° C. for 2 hours. The residue was poured onto ice/water. Extracted into ethyl acetate (2×80 mL) and the combined organics washed with saturated brine (100 mL), dried over Na₂SO₄, filtered and evaporated to give crude product. The crude product was purified by flash silica chromatography, elution gradient 10 to 30% EtOAc in isohexane. Pure fractions were evaporated to dryness to afford the title compound (0.810 g, 48.3%) as a colourless oil. ¹H NMR (400 MHz, CDCl₃) 0.16 (3H, s), 0.19 (3H, s), 0.93 (9H, s), 1.92 (1H, dd), 2.29 (1H, dd), 3.07 3.15 (1H, m), 3.23 3.30 (1H, m), 4.36 (3H, dt), 7.01 (1H, dd), 7.33 (1H, d), 7.56 (1H, d) m/z (ES+) (M+H)+=418, 420

### Intermediate 30: (S)-3-(tert-Butyldimethylsilyloxy)pyrrolidin-2-one

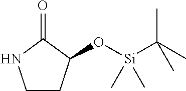

TBDMS-Cl (2.236 g, 14.84 mmol) was added to (S)-3-hydroxypyrrolidin-2-one (1.25 g, 12.36 mmol), N,N-dimethylpyridin-4-amine (0.060 g, 0.49 mmol) and imidazole (1.683 g, 24.73 mmol) in DCM (50 mL) under nitrogen. The resulting solution was stirred at RT for 16 hours. The reaction mixture was diluted with water extracted twice with DCM (50 mL). The organic layer was dried over MgSO₄, filtered and evaporated to afford crude product. The crude product was purified by flash silica chromatography, eluting with 50% EtOAc in isohexane. Pure fractions were evaporated to dryness to afford (S)-3-(tert-butyldimethylsilyloxy)pyrrolidin-2-one (1.960 g, 73.6%) as a white crystalline solid.

¹H NMR (400 MHz, CDCl₃) −0.02 (3H, d), −0.01-0.03 (3H, m), 0.75 (9H, s), 1.81-1.92 (1H, m), 2.21 (1H, dtd), 3.09 (1H, dt), 3.22 (1H, dddd), 4.10 (1H, t), 5.78 (1H, s).

### Intermediate 31: tert-Butyl 3-(((4′-(4-amino-5-oxo-7,8-dihydropyrimido[5,4-f][1,4]oxazepin-6(5H)-yl)-2-chlorobiphenyl-4-yl)methyl)(methyl)amino-3-oxopropylcarbamate

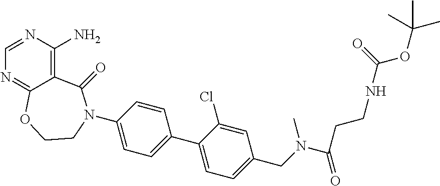

4-Amino-6-(4-bromophenyl)-7,8-dihydropyrimido[5,4-f][1,4]oxazepin-5(6H)-one (Intermediate 3; 0.14 g, 0.42 mmol), tert-butyl 3-((3-chloro-4-(4,4,5,5-tetramethyl-1,3,2-dioxaborolan-2-yl)benzyl)(methyl)amino)-3-oxopropylcarbamate (Intermediate 32; 0.284 g, 0.50 mmol), (1,1′-bis(diphenylphosphino)ferrocene)-dichloropalladium(II) (DCM adduct) (0.017 g, 0.02 mmol) and tripotassium phosphate (0.106 g, 0.50 mmol) were suspended in DME (3 mL), methanol (1.5 mL) and water (0.75 mL) and sealed into a microwave tube. The mixture was degassed under vacuum and the atmosphere replaced with nitrogen. The reaction was heated to 110° C. for 40 minutes in the microwave reactor and cooled to RT. The reaction mixture was evaporated to dryness and redissolved in methyl THF (100 mL), and washed sequentially with water (100 mL) and saturated brine (100 mL). A solid was filtered off and MeOH/DCM added to this and re-filtered. The filtrate was combined with the organic layer from above, filtered through a phase separating funnel and evaporated to afford crude product. The crude product was purified by flash silica chromatography, elution gradient 1 to 8% MeOH in DCM. Pure fractions were evaporated to dryness to afford the title compound (0.228 g, 94%) as a pale yellow gum. ¹H NMR (400 MHz, CDCl₃) 1.42 (9H, t), 2.53-2.67 (2H, m), 3.00 (3H, d), 3.49 (2H, d), 4.06-4.13 (2H, m), 4.58 (2H, d), 4.71-4.78 (2H, m), 5.34 (1H, s), 5.71 (1H, s), 7.09 7.21 (1H, m), 7.30-7.40 (4H, m), 7.53 (2H, d), 8.21 (1H, s), 8.30 (1H, s). m/z (ES+) M+=581

### Intermediate 32: tert-Butyl 3-((3-chloro-4-(4,4,5,5-tetramethyl-1,3,2-dioxaborolan-2-yl)benzyl)(methyl)amino)-3-oxopropylcarbamate

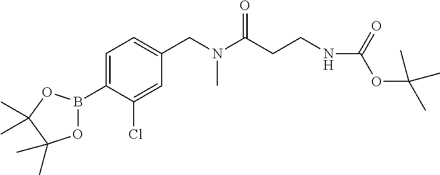

tert-Butyl 3-((4-bromo-3-chlorobenzyl)(methyl)amino)-3-oxopropylcarbamate (Intermediate 33; 0.47 g, 1.16 mmol), bis(pinacolato)diboron (0.441 g, 1.74 mmol), potassium acetate (0.352 g, 3.59 mmol) and (1,1′-bis(diphenylphosphino)ferrocene)-dichloropalladium(II) (DCM adduct) (0.057 g, 0.07 mmol) were suspended in dioxane (20 mL) and sealed into a microwave tube. The suspension was degassed by evacuation and inlet of nitrogen. The reaction was heated to 120° C. for 45 minutes in the microwave reactor and cooled to RT, evaporated and the residue suspended in EtOAc and water. This was filtered through celite and the residue evaporated. The crude product was purified by flash silica chromatography, elution gradient 1 to 7% MeOH in DCM. Pure fractions were evaporated to dryness to afford the title compound (0.300 g, 57.2%) as a colourless oil. ¹H NMR (400 MHz, CDCl₃) δ 1.36 (12H, d), 1.43 (9H, d), 2.45-2.61 (2H, m), 2.86-2.96 (3H, m), 3.40-3.51 (2H, m), 4.46-4.57 (2H, m), 5.33 (1H, s), 6.98-7.10 (1H, m), 7.12-7.22 (1H, m), 7.66 (1H, dd). m/z (ES+) (M+H)+=453

### Intermediate 33: tert-Butyl 3-((4-bromo-3-chlorobenzyl)(methyl)amino)-3-oxopropylcarbamate

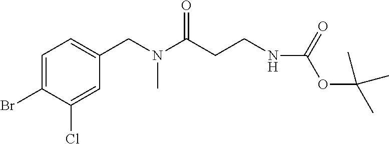

O-(7-Azabenzotriazol-1-yl)-N,N,N′,N′-tetramethyluronium hexafluorophosphate (0.778 g, 2.05 mmol) was added portionwise to 1-(4-bromo-3-chlorophenyl)-N-methylmethanamine (Intermediate 16; 0.4 g, 1.71 mmol), 3-(tert-butoxycarbonylamino)propanoic acid (0.323 g, 1.71 mmol) and DIPEA (1.188 mL, 6.82 mmol) in DMF (10 mL) at RT under nitrogen. The resulting solution was stirred at RT for 20 hours. The reaction mixture was diluted with EtOAc (50 mL), and washed sequentially with saturated NaHCO₃ (25 mL), saturated brine (25 mL), and water (25 mL). The organic layer was dried over MgSO₄, filtered and evaporated to afford crude product. The crude product was purified by flash silica chromatography, elution gradient 0 to 5% MeOH in DCM. Fractions were evaporated to dryness to afford tert-butyl 3-((4-bromo-3-chlorobenzyl)(methyl)amino)-3-oxopropylcarbamate (0.475 g, 68.6%) as a brown oil. ¹H NMR (400 MHz, CDCl₃) δ 1.43 (9H, d), 2.48-2.61 (2H, m), 2.91-2.96 (3H, m), 3.42-3.50 (2H, m), 4.43-4.53 (2H, m), 5.27 (1H, s), 6.86-7.02 (1H, m), 7.22-7.32 (1H, m), 7.53-7.62 (1H, m). m/z (ES+) (M-Boc)=307, 309

### Intermediate 34 tert-Butyl 2-(((4′-(4-amino-5-oxo-7,8-dihydropyrimido[5,4-f][1,4]oxazepin-6(5H)-yl)-2-chlorobiphenyl-4-yl)methyl)methyl)amino-2-oxoethylcarbamate

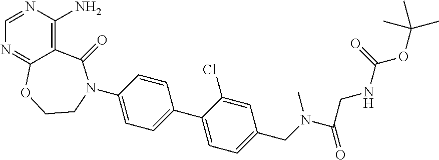

4-Amino-6-(4-bromophenyl)-7,8-dihydropyrimido[5,4-f][1,4]oxazepin-5(6H)-one (Intermediate 3; 0.27 g, 0.81 mmol), tert-butyl 2-((3-chloro-4-(4,4,5,5-tetramethyl-1,3,2-dioxaborolan-2-yl)benzyl)(methyl)amino)-2-oxoethylcarbamate (Intermediate 35; 0.574 g, 1.05 mmol), (1,1′-bis(diphenylphosphino)ferrocene)-dichloropalladium(II) (DCM adduct) (0.033 g, 0.04 mmol) and tripotassium phosphate (0.205 g, 0.97 mmol) were suspended in DME (3 mL), methanol (1.5 mL) and water (0.75 mL) and sealed into a microwave tube. The mixture was degassed under vacuum and the atmosphere replaced with nitrogen. The reaction was heated to 110° C. for 40 minutes in the microwave reactor and cooled to RT. The reaction mixture was evaporated to dryness and redissolved in methyl THF (100 mL), and washed sequentially with water (100 mL) and saturated brine (100 mL). A solid was filtered off and MeOH/DCM added to this and re-filtered. The filtrate was combined with the organic layer from above, filtered through a phase separating funnel and evaporated to afford crude product. The crude product was purified by flash silica chromatography, elution gradient 1 to 8% MeOH in DCM. Pure fractions were evaporated to dryness to afford the title compound (0.373 g, 82%) as a pale yellow foam. ¹H NMR (400 MHz, CDCl₃) δ 1.46 (9H, d), 3.00 (3H, d), 4.01-4.07 (2H, m), 4.07-4.12 (2H, m), 4.56 (2H, d), 4.70-4.77 (2H, m), 5.56 (1H, s), 5.66 (1H, s), 7.10-7.23 (1H, m), 7.29-7.40 (4H, m), 7.51-7.57 (2H, m), 8.20 (1H, s), 8.31 (1H, s). m/z (ES+) M+=567

### Intermediate 35: tert-Butyl 2-((3-chloro-4-(4,4,5,5-tetramethyl-1,3,2-dioxaborolan-2-yl)benzyl)(methyl)amino)-2-oxoethylcarbamate

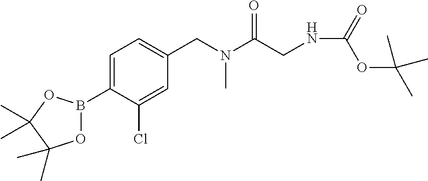

tert-Butyl 2-((4-bromo-3-chlorobenzyl)(methyl)amino)-2-oxoethylcarbamate (Intermediate 36; 0.63 g, 1.61 mmol), bis(pinacolato)diboron (0.613 g, 2.41 mmol), potassium acetate (0.489 g, 4.99 mmol) and (1,1′-bis(diphenylphosphino)ferrocene)-dichloropalladium(II) (DCM adduct) (0.079 g, 0.10 mmol) were suspended in dioxane (20 mL) and sealed into a microwave tube. The suspension was degassed by evacuation and inlet of nitrogen. The reaction was heated to 120° C. for 45 minutes in the microwave reactor and cooled to RT, evaporated and the residue suspended in EtOAc and water. This was filtered through celite and the residue evaporated. The crude product was purified by flash silica chromatography, elution gradient 1 to 70% MeOH in DCM. Pure fractions were evaporated to dryness to afford the title compound (0.580 g, 82%) as a colourless oil. ¹H NMR (400 MHz, CDCl₃) δ 1.37 (12H, d), 1.43-1.48 (9H, m), 2.82-2.99 (3H, m), 3.98-4.03 (2H, m), 4.42-4.59 (2H, m), 5.54 (1H, d), 6.99 7.11 (1H, m), 7.17 (1H, d), 7.67 (1H, dd). m/z (ES+) M+=

### Intermediate 36: tert-Butyl 2-((4-bromo-3-chlorobenzyl)(methyl)amino)-2-oxoethylcarbamate

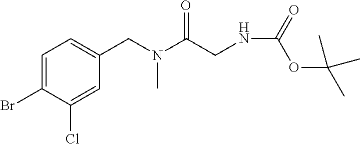

O-(7-Azabenzotriazol-1-yl)-N,N,N′,N′-tetramethyluronium hexafluorophosphate (0.778 g, 2.05 mmol) was added portionwise to 1-(4-bromo-3-chlorophenyl)-N-methylmethanamine (Intermediate 16; 0.4 g, 1.71 mmol), 2-(tert-butoxycarbonylamino)acetic acid (0.299 g, 1.71 mmol) and DIPEA (1.188 mL, 6.82 mmol) in DMF (10 mL) at RT under nitrogen. The resulting solution was stirred at RT for 20 hours. The reaction mixture was diluted with EtOAc (50 mL), and washed sequentially with saturated NaHCO₃ (25 mL), saturated brine (25 mL), and water (25 mL). The organic layer was dried over MgSO₄, filtered and evaporated to afford crude product. The crude product was purified by flash silica chromatography, elution gradient 0 to 5% MeOH in DCM. Fractions were evaporated to dryness to afford the title compound (0.640 g, 96%) as a brown oil. ¹H NMR (400 MHz, CDCl₃) δ 1.43-1.48 (9H, m), 2.84 (3H, d), 4.01 (2H, d), 4.47 (2H, d), 5.49 (1H, s), 6.88-7.02 (1H, m), 7.33 (1H, d), 7.59 (1H, dd). m/z (ES+) (M-Boc)=291, 293

### Intermediate 37: tert-Butyl 4-(((4′-(4-amino-5-oxo-7,8-dihydropyrimido[5,4-f][1,4]oxazepin-6(5H)-yl)-2-chlorobiphenyl-4-yl)methyl)(methyl)amino)-4-oxobutylcarbamate

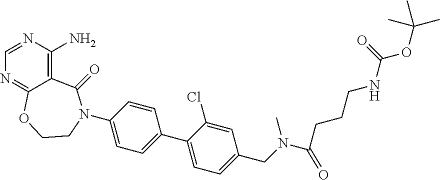

4-Amino-6-(4-bromophenyl)-7,8-dihydropyrimido[5,4-f][1,4]oxazepin-5(6H)-one (Intermediate 3; 0.13 g, 0.39 mmol), tert-butyl 4-((3-chloro-4-(4,4,5,5-tetramethyl-1,3,2-dioxaborolan-2-yl)benzyl)(methyl)amino)-4-oxobutylcarbamate (Intermediate 38; 0.217 g, 0.47 mmol), (1,1′-bis(diphenylphosphino)ferrocene)-dichloropalladium(II) (DCM adduct) (0.016 g, 0.02 mmol) and tripotassium phosphate (0.099 g, 0.47 mmol) were suspended in DME (3 mL), methanol (1.5 mL) and water (0.75 mL) and sealed into a microwave tube. The mixture was degassed under vacuum and the atmosphere replaced with nitrogen. The reaction was heated to 110° C. for 40 minutes in the microwave reactor and cooled to RT. The reaction mixture was evaporated to dryness and redissolved in methyl THF (100 mL), and washed sequentially with water (100 mL) and saturated brine (100 mL). A solid was filtered off and MeOH/DCM added to this and re-filtered. The filtrate was combined with the organic layer from above, filtered through a phase separating funnel and evaporated to afford crude product. The crude product was purified by flash silica chromatography, elution gradient 1 to 8% MeOH in DCM. Pure fractions were evaporated to dryness to afford the title compound (0.060 g, 26.0%) as a pale yellow gum. ¹H NMR (400 MHz, CDCl₃) 1.41-1.46 (9H, m), 1.86-1.92 (2H, m), 2.44 (2H, dt), 3.00 (3H, s), 3.21 (2H, dt), 4.06-4.11 (2H, m), 4.54-4.62 (2H, m), 4.71-4.76 (2H, m), 5.71 (1H, s), 7.10-7.22 (1H, m), 7.29-7.39 (4H, m), 7.50-7.56 (3H, m), 8.17 (1H, s), 8.30 (1H, s). m/z (ES+) M+=595

### Intermediate 38: tert-Butyl 4-((3-chloro-4-(4,4,5,5-tetramethyl-1,3,2-dioxaborolan-2-yl)benzyl)(methyl)amino)-4-oxobutylcarbamate

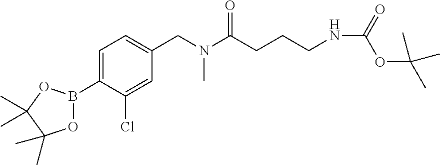

(1,1′-Bis(diphenylphosphino)ferrocene)-dichloropalladium(II) (DCM adduct) (0.039 g, 0.05 mmol) was added to tert-butyl 4-((4-bromo-3-chlorobenzyl)(methyl)amino)-4-oxobutylcarbamate (Intermediate 39; 0.33 g, 0.79 mmol), bis(pinacolato)diboron (0.299 g, 1.18 mmol) and potassium acetate (0.239 g, 2.44 mmol) in dioxane (15 mL). The suspension was degassed by evacuation and inlet of nitrogen and then heated, under nitrogen, to reflux overnight. The reaction mixture was allowed to cool, evaporated and the residue suspended in EtOAc and water. This was filtered through celite and the residue evaporated. The crude product was purified by flash silica chromatography, elution gradient 1 to 10% MeOH in DCM. Pure fractions were evaporated to dryness to afford the title compound (0.230 g, 62.7%) as a colourless oil. ¹H NMR (400 MHz, CDCl₃) 1.22-1.37 (12H, m), 1.43 (9H, d), 1.79-1.93 (2H, m), 2.32-2.47 (2H, m), 2.87-2.97 (3H, m), 3.19 (2H, dt), 4.41-4.59 (2H, m), 4.81 (1H, s), 6.98-7.16 (1H, m), 7.19-7.26 (1H, m), 7.54-7.71 (1H, m). m/z (ES+) (M+H)+=467

### Intermediate 39: tert-Butyl 4-((4-bromo-3-chlorobenzyl)(methyl)amino)-4-oxobutylcarbamate

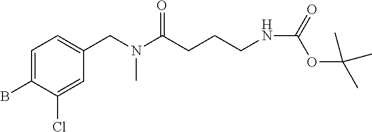

O-(7-Azabenzotriazol-1-yl)-N,N,N′,N′-tetramethyluronium hexafluorophosphate (0.778 g, 2.05 mmol) was added portionwise to 1-(4-bromo-3-chlorophenyl)-N-methylmethanamine (Intermediate 16; 0.4 g, 1.71 mmol), 4-(tert-butoxycarbonylamino)butanoic acid (0.347 g, 1.71 mmol) and DIPEA (1.188 mL, 6.82 mmol) in DMF (10 mL) at RT under nitrogen. The resulting solution was stirred at RT for 20 hours. The reaction mixture was diluted with EtOAc (50 mL), and washed sequentially with saturated NaHCO₃ (25 mL), saturated brine (25 mL), and water (25 mL). The organic layer was dried over MgSO₄, filtered and evaporated to afford crude product. The crude product was purified by flash silica chromatography, elution gradient 0 to 8% MeOH in DCM. Fractions were evaporated to dryness to afford tert-butyl 4-((4-bromo-3-chlorobenzyl)(methyl)amino)-4-oxobutylcarbamate (0.330 g, 46.1%) as a brown oil. ¹H NMR (400 MHz, CDCl₃) 1.43 (9H, s), 1.78-1.94 (2H, m), 2.33-2.47 (2H, m), 2.94 (3H, s), 3.09-3.24 (2H, m), 4.49 (2H, s), 4.84 (1H, s), 7.01 (1H, dd), 7.33 (1H, d), 7.55 (1H, dd). m/z (ES+) (M+H)+=419, 421

### Intermediate 40: 4-Amino-6-(4-bromophenyl)-2-methyl-7,8-dihydropyrimido[5,4-f][1,4]oxazepin-5(6H)-one

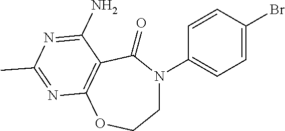

0.5M Ammonia in 1,4-dioxane (40 mL) was added to 6-(4-bromophenyl)-4-chloro-2-methyl-7,8-dihydropyrimido[5,4-f][1,4]oxazepin-5(6H)-one (Intermediate 41; 0.3 g, 0.81 mmol) at 20° C. The resulting suspension was stirred at 50° C. for 2 days. The reaction mixture was filtered to give a solid (not product). The crude solid was triturated with ether to give a solid which was collected by filtration and dried under vacuum to give the title compound (0.280 g, 99%) as a white solid. ¹H NMR (400 MHz, DMSO) δ 2.27 (3H, s), 3.93-3.99 (2H, m), 4.52-4.58 (2H, m), 7.31-7.36 (2H, m), 7.54 (2H, s), 7.58-7.63 (2H, m).

m/z (ES+) (M+H)+=349, 351

### Intermediate 41: 6-(4-Bromophenyl)-4-chloro-2-methyl-7,8-dihydropyrimido[5,4-f][1,4]oxazepin-5(6H)-one

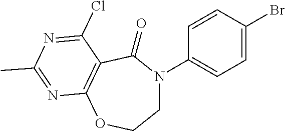

Triethylamine (0.652 mL, 4.69 mmol) was added in one portion to N-(4-bromophenyl)-4,6-dichloro-N-(2-hydroxyethyl)-2-methylpyrimidine-5-carboxamide (Intermediate 42; 0.5 g, 1.23 mmol) in acetonitrile (10 mL) at 20° C. under nitrogen. The resulting solution was stirred at 80° C. for 6 hours. The reaction mixture was evaporated to dryness and redissolved in EtOAc (500 mL), and washed with water (3×100 mL) and saturated brine (100 mL). The organic layer was dried over Na₂SO₄, filtered and evaporated to afford crude product. The crude solid was triturated with MeOH (50 mL) to give a solid which was collected by filtration, washed with MeOH (25 mL) and ether (50 mL) and dried under vacuum to give the title compound (0.300 g, 65.9%) as a white solid. ¹H NMR (400 MHz, DMSO) δ 2.63 (3H, s), 4.17-4.23 (2H, m), 4.73-4.78 (2H, m), 7.46-7.51 (2H, m), 7.69-7.74 (2H, m). m/z (ES+) (M+H)+=368, 370

### Intermediate 42: N-(4-Bromophenyl)-4,6-dichloro-N-(2-hydroxyethyl)-2-methylpyrimidine-5-carboxamide

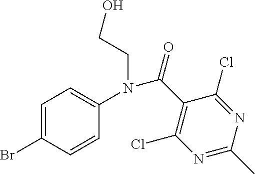

Concentrated hydrochloric acid (0.2 mL) was added to N-(4-bromophenyl)-N-(2-(tert-butyldimethylsilyloxy)ethyl)-4,6-dichloro-2-methylpyrimidine-5-carboxamide (Intermediate 43; 0.78 g, 1.50 mmol) in methanol (6 mL) and stirred for 30 minutes. The reaction mixture was neutralised with saturated NaHCO₃ and evaporated to dryness and redissolved in EtOAc (150 mL), and washed sequentially with saturated NaHCO₃ (100 mL) and saturated brine (100 mL). The organic layer was dried over Na₂SO₄, filtered and evaporated to afford crude product. The crude product was purified by flash silica chromatography, elution gradient 10 to 80% EtOAc in isohexane. Pure fractions were evaporated to dryness to afford N-(4-bromophenyl)-4,6-dichloro-N-(2-hydroxyethyl)-2-methylpyrimidine-5-carboxamide (0.492 g, 81%) as a colourless oil which solidified on standing. ¹H NMR (400 MHz, CDCl₃) δ 2.05 (1H, t), 2.61 (3H, s), 3.92 (2H, q), 4.06 (2H, t), 7.28-7.33 (2H, m), 7.41-7.44 (2H, m). m/z (ES+) (M+H)+=406

### Intermediate 43: N-(4-bromophenyl)-N-(2-(tert-butyldimethylsilyloxy)ethyl)-4,6-dichloro-2-methylpyrimidine-5-carboxamide

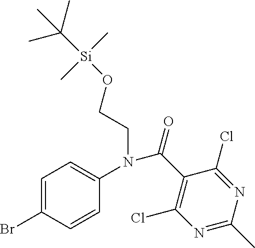

4,6-Dichloro-2-methylpyrimidine-5-carbonyl chloride (Intermediate 44; 1.003 g, 4.45 mmol) as a solution in THF (8.75 mL) was added dropwise to 4-bromo-N-(2-(tert-butyldimethylsilyloxy)ethyl)aniline (1.4 g, 4.24 mmol) and triethylamine (1.767 mL, 12.71 mmol) in THF (35 mL) at 0° C. over a period of 10 minutes under nitrogen. The resulting solution was stirred at 20° C. for 70 hours under nitrogen. The reaction mixture was evaporated to dryness and redissolved in EtOAc (250 mL), and washed sequentially with water (2×150 mL) and saturated brine (75 mL). The organic layer was dried over Na₂SO₄, filtered and evaporated to afford crude product. The crude product was purified by flash silica chromatography, elution gradient 5 to 40% EtOAc in isohexane. Pure fractions were evaporated to dryness to afford the title compound (0.780 g, 35.4%) as a colourless oil. ¹H NMR (400 MHz, CDCl₃) δ −0.00 (6H, s), 0.82 (9H, s), 2.55 (3H, s), 3.85 (2H, t), 3.93 (2H, t), 7.22-7.26 (2H, m), 7.30-7.35 (2H, m). m/z (ES+) (M+H)+=520

### Intermediate 44: 4,6-Dichloro-2-methylpyrimidine-5-carbonyl chloride

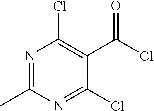

Sulfuryl chloride (0.114 mL, 1.42 mmol) and 2,2′-azobisisobutyronitrile (6.88 mg, 0.04 mmol) were added to a solution of 4,6-dichloro-2-methylpyrimidine-5-carbaldehyde (Intermediate 45; 0.16 g, 0.84 mmol) in CCl₄ (3 mL). The reaction was heated at reflux for 3 hours then cooled. The rm was injected directly onto a column. The crude product was purified by flash silica chromatography, eluting with DCM. Pure fractions were evaporated to dryness to afford the title compound (0.180 g, 95%) as a colourless oil. ¹H NMR (400 MHz, CDCl₃) δ 2.77 (3H, s). m/z (ES+) M-COCl—H=161

### Intermediate 45: 4,6-Dichloro-2-methylpyrimidine-5-carbaldehyde

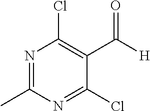

Water (0.199 mL, 11.03 mmol) was added to phosphoryl trichloride (7.28 mL, 78.11 mmol) over an ice bath. N,N-dimethylaniline (0.291 mL, 2.30 mmol) was then added. N-((4,6-dihydroxy-2-methylpyrimidin-5-yl)methylene)-N-methylmethanaminium chloride (Intermediate 46; 1 g, 4.59 mmol) was added portionwise to the resulting solution at room temperature. The resulting suspension was stirred at 120° C. for 20 hours. The reaction mixture was poured slowly into ice/chloroform. The chloroform layer was washed sequentially with saturated NaHCO₃ (200 mL) and saturated brine (100 mL). The organic layer was dried over Na₂SO₄, filtered and evaporated to afford crude product. The crude product was purified by flash silica chromatography, elution gradient 5 to 10% EtOAc in isohexane. Pure fractions were evaporated to dryness to afford the title compound (0.160 g, 18.23%) as a white solid. ¹H NMR (400 MHz, CDCl₃) δ 2.77 (3H, s), 10.44 (1H, s).

### Intermediate 46: N-((4,6-Dihydroxy-2-methylpyrimidin-5-yl)methylene)-N-methylmethanaminium chloride

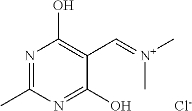

2-Methylpyrimidine-4,6-diol (2 g, 15.86 mmol) was added portionwise to N-(chloromethylene)-N-methylmethanaminium chloride (3.45 g, 26.96 mmol) in chloroform (15 mL) at 20° C. over a period of 10 minutes. The resulting suspension was stirred at 55° C. for 3 hours and stirred at room temperature overnight. The reaction mixture was filtered and washed with chloroform then ether and dried on the rotavapor to give the title compound (3.11 g, 90%) as a yellow solid. ¹H NMR (400 MHz, D₂O) δ 2.51 (1H, d), 2.57 (3H, s), 2.69 (1H, s), 3.37 (3H, s), 3.63 (3H, s), 8.39 (1H, d). m/z (ES+) (M+H)+=182

### Intermediate 47a: 1-(3-Chloro-4-(4,4,5,5-tetramethyl-1,3,2-dioxaborolan-2-yl)benzyl)pyrimidin-2(1H)-one

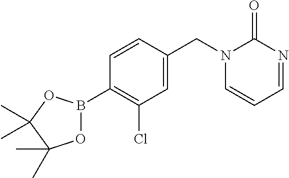

1-(4-Bromo-3-chlorobenzyl)pyrimidin-2(1H)-one (Intermediate 47b; 0.68 g, 2.27 mmol), potassium acetate (0.668 g, 6.81 mmol), (1,1′-bis(diphenylphosphino)ferrocene)-dichloropalladium(II) (DCM adduct) (0.130 g, 0.16 mmol) and bis(pinacolato)diboron (0.692 g, 2.72 mmol) were suspended in a microwave tube. The suspension was degassed. The reaction was heated to 130° C. for 2 hours in the microwave reactor and cooled to RT. The reaction mixture was evaporated and the residue taken up in Methanol and filtered. The filtrate was evaporated and partitioned between water (80 mL) and methyl THF (100 mL). The organic phase was separated and evaporated. The crude product was purified by flash silica chromatography, elution gradient 1 to 10% MeOH in EtOAc. Pure fractions were evaporated to dryness to afford the title compound (0.337 g, 42.8%) as a yellow solid

¹H NMR (400 MHz, CDCl₃) δ 1.36 (12H, s), 5.07 (2H, s), 6.24-6.30 (1H, m), 7.21 (1H, dd), 7.28-7.35 (1H, m), 7.57 (1H, dd), 7.69 (1H, d), 8.53-8.63 (1H, m). m/z (ES+) (M+H)+=347

### Intermediate 47b: 1-(4-Bromo-3-chlorobenzyl)pyrimidin-2(1H)-one

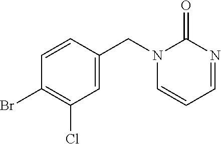

4-Bromo-3-chlorobenzyl methanesulfonate (Intermediate 13; 1.2 g, 4.01 mmol) was added to pyrimidin-2-ol hydrochloride (0.637 g, 4.81 mmol) and potassium carbonate (1.384 g, 10.01 mmol) in DMF (20 mL) under nitrogen. The resulting suspension was stirred at 80° C. for 1 hour. The reaction mixture was diluted with EtOAc (30 mL), and washed sequentially with water (3×10 mL) and saturated brine (10 mL) and evaporated to afford crude product. The crude product was purified by flash silica chromatography, elution gradient 1 to 10% MeOH in DCM. Pure fractions were evaporated to dryness to afford the title compound (0.690 g, 57.5%) as a white solid. ¹H NMR (400 MHz, DMSO) δ 5.01 (2H, s), 6.47 (1H, dd), 7.23 (1H, dd), 7.62 (1H, d), 7.75 (1H, d), 8.36 (1H, dd), 8.57 (1H, dd). m/z (ES+) (M+H)+=299, 301

### Intermediate 48: (R)-4-(4-Amino-8-methyl-5-oxo-7,8-dihydropyrimido[5,4-f][1,4]oxazepin-6(5H)-yl)phenyl trifluoromethanesulfonate

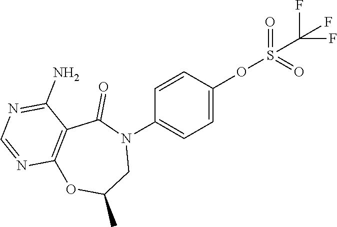

Potassium carbonate (0.753 g, 5.45 mmol) was added to (R)-4-amino-6-(4-hydroxyphenyl)-8-methyl-7,8-dihydropyrimido[5,4-f][1,4]oxazepin-5(6H)-one (Intermediate 48a; 0.52 g, 1.82 mmol) and 1,1,1-trifluoro-N-phenyl-N-(trifluoromethylsulfonyl)methanesulfonamide (0.649 g, 1.82 mmol) in THF (50 mL) at 20° C. The resulting suspension was stirred at 20° C. for 3 days. The reaction mixture was evaporated to dryness and redissolved in EtOAc (25 mL), and washed sequentially with water (25 mL) and saturated brine (25 mL). The organic layer was dried over Na₂SO₄, filtered and evaporated to afford crude product. The crude product was purified by flash silica chromatography, elution gradient 1 to 8% MeOH in DCM. Pure fractions were evaporated to dryness to afford the title compound (0.466 g, 61.3%) as a white foam.

¹H NMR (400 MHz, CDCl₃) δ 1.48 (3H, d), 3.88 (2H, qd), 4.87-4.97 (1H, m), 5.61 (1H, s), 7.33-7.43 (4H, m), 7.93 (1H, s), 8.32 (1H, s). m/z (ES+) (M+H)+=419

### Intermediate 48a: (R)-4-Amino-6-(4-hydroxyphenyl)-8-methyl-7,8-dihydropyrimido[5,4-f][1,4]oxazepin-5(6H)-one

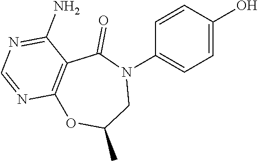

(R)-4-Amino-6-(4-(benzyloxy)phenyl)-8-methyl-7,8-dihydropyrimido[5,4-f][1,4]oxazepin-5(6H)-one (Intermediate 48b; 0.927 g, 2.46 mmol) was dissolved in MeOH (100 mL) followed by addition of dihydroxypalladium (0.346 g, 0.49 mmol). The reaction mixture was put under H₂ atmosphere (1 atm) and stirred at room temperature for 1 hour. The catalyst was filtered off and rinsed with MeOH. The solvent was removed under reduced pressure to give the title compound (0.520 g, 73.8%) as a beige solid. ¹H NMR (400 MHz, DMSO) δ 1.27 (3H, d), 3.77 (2H, d), 4.80-4.97 (1H, m), 6.72-6.84 (2H, m), 7.08-7.19 (2H, m), 7.45 (2H, s), 8.18 (1H, s), 9.52 (1H, s). m/z (ES+) (M+H)+=287

### Intermediate 48b: (R)-4-Amino-6-(4-(benzyloxy)phenyl)-8-methyl-7,8-dihydropyrimido[5,4-f][1,4]oxazepin-5(6H)-one

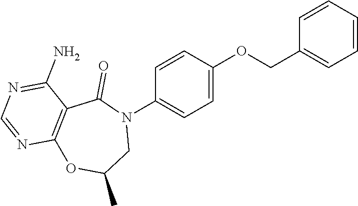

0.5M Ammonia in 1,4-dioxane (100 mL) was added to (R)-6-(4-(benzyloxy)phenyl)-4-chloro-8-methyl-7,8-dihydropyrimido[5,4-f][1,4]oxazepin-5(6H)-one (Intermediate 48c; 1.09 g, 2.75 mmol) at 20° C. The resulting suspension was stirred at 55° C. for 2 days. The reaction mixture was filtered to give a non-product solid which was discarded. The filtrate was evaporated and the residual crude solid was triturated with ether to give a solid which was collected by filtration and dried under vacuum to give the title compound (0.927 g, 89%) as a beige solid. ¹H NMR (400 MHz, DMSO) δ 1.27 (3H, d), 3.79-3.82 (2H, m), 4.89 (1H, td), 5.13 (2H, s), 7.02-7.08 (2H, m), 7.24-7.47 (9H, m), 8.19 (1H, s). m/z (ES+) (M+H)+=377

### Intermediate 48c: (R)-6-(4-(Benzyloxy)phenyl)-4-chloro-8-methyl-7,8-dihydropyrimido[5,4-f][1,4]oxazepin-5(6H)-one

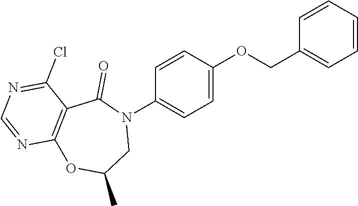

(R)—N-(4-(Benzyloxy)phenyl)-4,6-dichloro-N-(2-hydroxypropyl)pyrimidine-5-carboxamide (Intermediate 48d; 2.6 g, 6.01 mmol) and potassium carbonate (2.078 g, 15.04 mmol) were suspended in acetonitrile (20 mL) and sealed into a microwave tube. The reaction was heated to 80° C. for 24 hours in the microwave reactor and cooled to RT. Heated to 80° C. for a further 22 hours in the microwave reactor and cooled to RT. Heated to 85° C. for a further 20 hours in the microwave reactor and cooled to RT. The reaction mixture was evaporated to dryness and redissolved in DCM (75 mL) and washed with water (75 mL). The organic layer was dried over Na₂SO₄, filtered and evaporated to afford crude product. The crude product was purified by flash silica chromatography, elution gradient 20 to 80% EtOAc in isohexane. Pure fractions were evaporated to dryness to afford the title compound (1.090 g, 45.8%) as a yellow solid. ¹H NMR (400 MHz, DMSO) δ 1.28 (3H, d), 3.85-4.07 (2H, m), 5.04-5.10 (1H, m), 5.14 (2H, s), 7.03-7.15 (2H, m), 7.28-7.49 (7H, m), 8.83 (1H, s). m/z (ES+) (M+H)+=396

### Intermediate 48d: (R)—N-(4-(benzyloxy)phenyl)-4,6-dichloro-N-(2-hydroxypropyl)pyrimidine-5-carboxamide

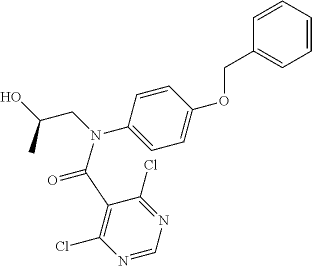

A solution of (R)—N-(4-(benzyloxy)phenyl)-N-(2-(tert-butyldimethylsilyloxy)propyl)-4,6-dichloropyrimidine-5-carboxamide (Intermediate 48e; 4.2 g, 7.68 mmol) in 4.0 M HCl in dioxane (50 mL) was stirred at 20° C. for 45 minutes. The reaction mixture was evaporated to dryness and redissolved in EtOAc (150 mL), and washed sequentially with saturated NaHCO₃ (100 mL) and saturated brine (100 mL). The organic layer was separated and filtered through a phase separating funnel and evaporated to afford an orange gum. The crude gum was triturated with EtOAc and isohexane to give a solid which was collected by filtration and dried under vacuum to give an orange solid (not product). The filtrate was evaporated to give the title compound (2.65 g, 80%) as an orange gum. This was used without further purification. ¹H NMR (400 MHz, DMSO, 30° C.) δ 1.20 (3H, d), 3.65 3.79 (1H, m), 3.84 (2H, ddt), 4.81 (1H, d), 5.06 (2H, s), 6.89-7.05 (2H, m), 7.28-7.51 (7H, m), 8.85 (1H, s). m/z (ES+) (M+H)+=432

### Intermediate 48e: (R)—N-(4-(Benzyloxy)phenyl)-N-(2-(tert-butyldimethylsilyloxy)propyl)-4,6-dichloropyrimidine-5-carboxamide

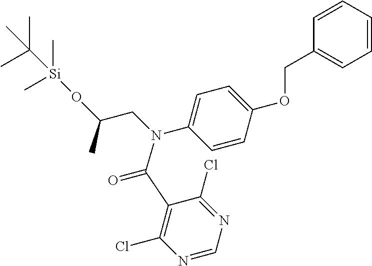

4,6-Dichloropyrimidine-5-carbonyl chloride (1.792 g, 6.78 mmol) as a solution in THF (25 mL) was added dropwise to (R)-4-(benzyloxy)-N-(2-(tert-butyldimethylsilyloxy)propyl)aniline (Intermediate 48f; 2.4 g, 6.46 mmol) and triethylamine (1.804 mL, 12.98 mmol) in THF (100 mL) at 0° C. over a period of 10 minutes under nitrogen. The resulting solution was stirred at 20° C. for 16 hours. The reaction mixture was evaporated to dryness and redissolved in EtOAc (250 mL), and washed sequentially with water (2×150 mL) and saturated brine (75 mL). The organic layer was dried over Na₂SO₄, filtered and evaporated to afford crude product (R)—N-(4-(benzyloxy)phenyl)-N-(2-(tert-butyldimethylsilyloxy)propyl)-4,6-dichloropyrimidine-5-carboxamide (4.2 g) as an orange oil. ¹H NMR (400 MHz, DMSO, 30° C.) δ −0.04 (3H, s), −0.00 (3H, s), 0.78 (9H, d), 1.26 (3H, d), 3.86-3.94 (2H, m), 3.99-4.13 (1H, m), 5.07 (2H, s), 6.89-7.04 (2H, m), 7.27-7.46 (7H, m), 8.87 (1H, s). m/z (ES+) (M+H)+=546

### Intermediate 48f: (R)-4-(Benzyloxy)-N-(2-(tert-butyldimethylsilyloxy)propyl)aniline

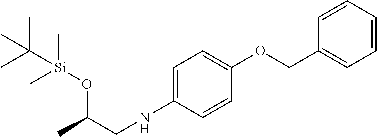

Pd(OAc)₂ (0.768 g, 3.42 mmol) and dicyclohexyl(2′,4′,6′-triisopropylbiphenyl-2-yl)phosphine (1.631 g, 3.42 mmol) were added in one portion to a degassed solution of 1-(benzyloxy)-4-bromobenzene (9.00 g, 34.20 mmol), (R)-2-(tert-butyldimethylsilyloxy)propan-1-amine (Intermediate 48 g; 7.77 g, 41.04 mmol) and cesium carbonate (16.72 g, 51.31 mmol) in toluene (200 mL) at 20° C. in a 300 mL Milestone microwave reactor. The microwave reactor was sealed and the reaction was heated to 120° C. for 10 hours in and cooled to RT. A second aliquot of catalyst was added and the reaction continued for 10 hours at 130° C. The reaction mixture was diluted with EtOAc (100 mL) and was filtered. The solid (inorganics and catalyst) was washed with ethylacete (50 mL). The filtrates were combined and washed with water (1×150 mL) and brine (1×150 mL). The crude product was evaporated and was purified by flash silica chromatography, elution gradient 0 to 30% EtOAc in isohexane. Pure fractions were evaporated to dryness to afford the title compound (2.40 g, 18.88%) as a orange oil. ¹H NMR (400 MHz, DMSO, 30° C.) δ 0.01 (6H, d), 0.83 (9H, s), 1.09 (3H, d), 2.91 (2H, t), 3.88-3.96 (1H, m), 4.93 (2H, s), 6.45-6.52 (2H, m), 6.71-6.79 (2H, m), 7.24-7.42 (6H, m). m/z (ES+) (M+H)+=372

### Intermediate 48 g: (R)-2-(tert-butyldimethylsilyloxy)propan-1-amine

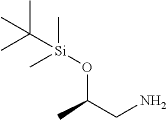

tert-Butyldimethylchlorosilane (20.07 g, 133.14 mmol) in DCM (125 mL) was added dropwise to a solution of (R)-1-aminopropan-2-ol (10 g, 133.14 mmol) and Triethylamine (37.1 mL, 266.28 mmol) in DCM (125 mL) cooled to 0° C. (ice water bath). Slight exotherm. The reaction mixture was allowed to warm to and stirred at room temperature overnight. The reaction mixture was quenched with saturated NH₄Cl (125 mL) extracted with DCM (2×200 mL). The combined organics were washed with brine (200 mL), dried over Na₂SO₄, filtered and evaporated to afford (R)-2-(tert-butyldimethylsilyloxy)propan-1-amine (25.1 g, 100%) as a colourless oil. ¹H NMR (400 MHz, DMSO, 30° C.) δ −0.00 (6H, s), 0.80-0.83 (9H, m), 0.99 (3H, t), 1.52 (2H, bs), 2.39 (2H, dd), 3.59-3.69 (1H, m). m/z (ES+) M-Me=174

### Intermediate 49: (R)-4-Amino-6-(2′-chloro-4′-(methylthiomethyl)biphenyl-4-yl)-8-methyl-7,8-dihydropyrimido[5,4-f][1,4]oxazepin-5(6H)-one

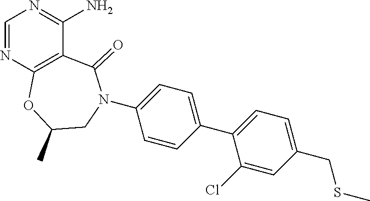

(R)-4-(4-amino-8-methyl-5-oxo-7,8-dihydropyrimido[5,4-f][1,4]oxazepin-6(5H)-yl)phenyl trifluoromethanesulfonate (Intermediate 48; 300 mg, 0.72 mmol), 2-(2-chloro-4-(methylthiomethyl)phenyl)-4,4,5,5-tetramethyl-1,3,2-dioxaborolane (Intermediate 49a; 257 mg, 0.86 mmol) (1,1′-bis(diphenylphosphino)ferrocene)-dichloropalladium(II) (DCM adduct) (29.3 mg, 0.04 mmol) and potassium phosphate (0.071 mL, 0.86 mmol) were suspended in DME (3 mL), water (0.75 mL) and MeOH (1.5 mL) and sealed into a microwave tube. The mixture was degassed under nitrogen and the atmosphere repaced with nitrogen. The reaction was heated to 110° C. for 40 minutes in the microwave reactor and cooled to RT. The reaction mixture was evaporated to dryness and redissolved in methyl THF (100 mL), and washed sequentially with water (100 mL) and saturated brine (100 mL). The organic layer was dried over Na₂SO₄, filtered and evaporated to afford crude product. A solid was filtered off and dissolved in MeOH/DCM but contained no product by TLC. The crude product was purified by flash silica chromatography, elution gradient 10 to 70% 10% MeOH/DCM in DCM. Pure fractions were evaporated to dryness to afford the title compound (220 mg, 69.6%) as a orange solid which was used without further purification. ¹H NMR (400 MHz, CDCl₃, 30° C.) δ 1.51 (3H, d), 2.06 (3H, s), 3.68 (2H, s), 3.88-4.01 (2H, m), 4.93-5.03 (1H, m), 5.62 (1H, s), 7.30 (2H, dd), 7.33-7.38 (2H, m), 7.44 (1H, d), 7.51-7.57 (2H, m), 8.04 (1H, s), 8.32 (1H, s). m/z (ES+), (M+H)+=441

### Intermediate 49a: 2-(2-Chloro-4-(methylthiomethyl)phenyl)-4,4,5,5-tetramethyl-1,3,2-dioxaborolane

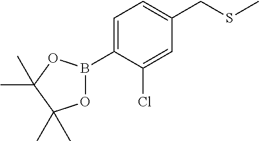

(4-Bromo-3-chlorobenzyl)(methyl)sulfane (1 g, 3.98 mmol), (1,1′-bis(diphenylphosphino)ferrocene)-dichloropalladium(II) (DCM adduct) (0.195 g, 0.24 mmol), bis(pinacolato)diboron (1.110 g, 4.37 mmol) and potassium acetate (0.770 mL, 12.32 mmol) were suspended in dioxane (20 mL), sealed into a microwave tube and degassed under vacuum. The reaction was heated to 130° C. for 45 minutes in the microwave reactor and cooled to RT. The reaction mixture was evaporated to dryness and redissolved in methyl THF (50 mL), and washed sequentially with water (50 mL) and saturated brine (50 mL). The organic layer was dried over Na₂SO₄, filtered and evaporated to afford crude product. The crude product was purified by flash silica chromatography, elution gradient 5 to 15% EtOAc in isohexane. Pure fractions were evaporated to dryness to afford the title compound (0.252 g, 21.23%) as a colourless oil.

¹H NMR (400 MHz, CDCl₃, 30° C.) δ 1.36 (12H, s), 1.96 (3H, s), 3.62 (2H, s), 7.17 (1H, dd), 7.30 (1H, d), 7.64 (1H, d).

### Intermediate 50: (R)-4-Chloro-6-(2′,6′-difluorobiphenyl-4-yl)-8-methyl-7,8-dihydropyrimido[5,4-f][1,4]oxazepin-5(6H)-one

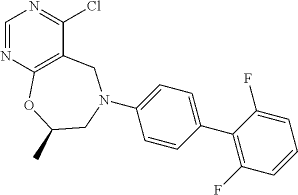

(R)-4,6-Dichloro-N-(2′,6′-difluorobiphenyl-4-yl)-N-(2-hydroxypropyl)pyrimidine-5-carboxamide (Intermediate 50a; 60 mg, 0.14 mmol) and potassium carbonate (47.3 mg, 0.34 mmol) were suspended in acetonitrile (5 mL) and sealed into a microwave tube. The reaction was heated to 100° C. for 16 hours in the microwave reactor and cooled to RT. The reaction mixture was evaporated to dryness and redissolved in methyl THF (75 mL), and washed with water (75 mL). The organic layer was dried over Na₂SO₄, filtered and evaporated to afford the title compound (53.0 mg, 96%) as a crude brown oil which was used without further purification. ¹H NMR (400 MHz, CDCl₃) δ 1.49 (3H, dd), 3.92-3.98 (2H, m), 5.06-5.16 (1H, m), 6.93-7.06 (2H, m), 7.27-7.37 (1H, m), 7.46-7.52 (2H, m), 7.58 (2H, d), 8.78 (1H, s). m/z (ES+) (M+H)+=402

### Intermediate 50a: (R)-4,6-Dichloro-N-(2′,6′-difluorobiphenyl-4-yl)-N-(2-hydroxypropyl)pyrimidine-5-carboxamide

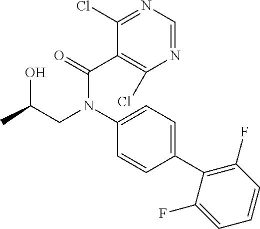

A solution of (R)—N-(2-(tert-butyldimethylsilyloxy)propyl)-4,6-dichloro-N-(2′,6′-difluorobiphenyl-4-yl)pyrimidine-5-carboxamide (Intermediate 50b; 0.33 g, 0.30 mmol) in 4.0 M HCl in dioxane (10 mL) was stirred at 20° C. for 45 minutes. The reaction mixture was evaporated to dryness and redissolved in DCM (150 mL), and washed sequentially with saturated NaHCO₃ (100 mL) and saturated brine (100 mL). The organic layer was separated and filtered through a phase separating funnel and evaporated to afford an orange gum. The crude product was purified by flash silica chromatography, elution gradient 10 to 40% EtOAc in isohexane. Pure fractions were evaporated to dryness to afford the title compound (0.060 g, 45.8%) as a colourless oil. ¹H NMR (400 MHz, CDCl₃, 30° C.) δ 1.31 (3H, dd), 2.23 (1H, s), 3.77-3.86 (1H, m), 4.08-4.24 (2H, m), 6.92-7.00 (2H, m), 7.29 (1H, ddd), 7.38-7.42 (2H, m), 7.46-7.52 (2H, m), 8.61 (1H, s). m/z (ES+) (M+H)+=438

### Intermediate 50b: (R)—N-(2-(tert-Butyldimethylsilyloxy)propyl)-4,6-dichloro-N-(2′,6′-difluorobiphenyl-4-yl)pyrimidine-5-carboxamide

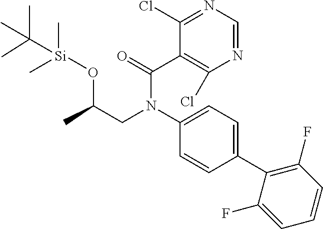

4,6-Dichloropyrimidine-5-carbonyl chloride (0.235 g, 0.83 mmol) as a solution in THF (2 mL) was added dropwise to (R)—N-(2-(tert-butyldimethylsilyloxy)propyl)-2′,6′-difluorobiphenyl-4-amine (Intermediate 50c; 0.3 g, 0.79 mmol) and triethylamine (0.133 mL, 0.95 mmol) in THF (10 mL) at 0° C. over a period of 5 minutes under nitrogen. The resulting solution was stirred at 20° C. for 3 days. The reaction mixture was evaporated to dryness and redissolved in EtOAc (250 mL), and washed sequentially with water (2×150 mL) and saturated brine (75 mL). The organic layer was dried over Na₂SO₄, filtered and evaporated to afford crude product. The crude product was purified by flash silica chromatography, elution gradient 5 to 10% EtOAc in isohexane. Product containing fractions were evaporated to dryness to afford the crude title compound (0.330 g, 75%) as a white solid. Taken on to the next stage without further purification.

¹H NMR (400 MHz, CDCl₃) δ −0.03-0.03 (3H, m), 0.04 (3H, s), 0.80 (9H, s), 1.38 (3H, d), 3.93-4.12 (2H, m), 4.19-4.29 (1H, m), 6.95-7.03 (2H, m), 7.31-7.51 (5H, m), 8.63 (1H, s). m/z (ES+) (M+H)+=552

### Intermediate 50c: (R)—N-(2-(tert-butyldimethylsilyloxy)propyl)-2′,6′-difluorobiphenyl-4-amine

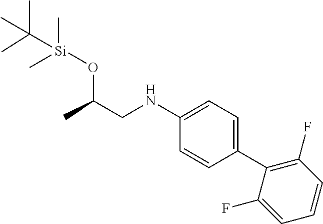

Pd(OAc)₂ (0.030 g, 0.13 mmol) and dicyclohexyl(2′,4′,6′-triisopropylbiphenyl-2-yl)phosphine (0.064 g, 0.13 mmol) were added in one portion to a degassed solution of 4′-bromo-2,6-difluorobiphenyl (Intermediate 50d; 0.36 g, 1.34 mmol), (R)-2-(tert-butyldimethylsilyloxy)propan-1-amine (0.355 g, 1.87 mmol) and cesium carbonate (0.654 g, 2.01 mmol) in toluene (10 mL) at 20° C. under nitrogen. The resulting suspension was stirred at 120° C. for 50 hours. The reaction mixture was diluted with EtOAc (150 mL) and water (150 mL) and the biphasic mixture was filtered through celite. The organic layer was separated and washed sequentially with water (150 mL) and saturated brine (150 mL). The organic layer was evaporated to afford crude product. The crude product was purified by flash silica chromatography, elution gradient 0 to 10% EtOAc in isohexane. Pure fractions were evaporated to dryness to afford the title compound (0.300 g, 59.4%) as a yellow solid. ¹H NMR (400 MHz, CDCl₃) δ 0.00 (6H, s), 0.83 (9H, s), 1.14 (3H, d), 2.92-2.99 (1H, m), 3.06-3.15 (1H, m), 3.94-4.02 (1H, m), 4.03-4.09 (1H, m), 6.55-6.61 (2H, m), 6.81-6.88 (2H, m), 7.05-7.13 (1H, m), 7.21 (2H, dt). m/z (ES+) (M+H)+=378

### Intermediate 50d: 4′-Bromo-2,6-difluorobiphenyl

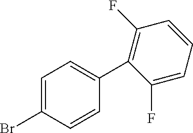

1-Bromo-4-iodobenzene (1.5 g, 5.30 mmol), potassium (2,6-difluorophenyl)trifluoroborate (1.166 g, 5.30 mmol), (1,1′-bis(diphenylphosphino)ferrocene)-dichloropalladium(II) (DCM adduct) (0.216 g, 0.27 mmol) and tripotassium phosphate (1.351 g, 6.36 mmol) were suspended in DME (10 mL), ethanol (5.0 mL) and water (2.5 mL) and sealed into a microwave tube. The mixture was degassed under vacuum and the atmosphere replaced with nitrogen. The reaction was heated to 110° C. for 60 minutes in the microwave reactor and cooled to RT. Evaporated and diluted with ethyl acetate (100 mL) and water (100 mL). Filtered through celite and the organic layer washed with brine (100 mL), dried over MgSO₄, filtered and evaporated to give crude product. The crude product was purified by flash silica chromatography, elution gradient 0 to 5% EtOAc in isohexane. Product containing fractions were evaporated to dryness to afford crude 4′-bromo-2,6-difluorobiphenyl (0.360 g, 25.2%) as a white solid. Taken on to the next stage without further purification. ¹H NMR (400 MHz, CDCl₃) δ 6.96-7.01 (2H, m), 7.26-7.32 (1H, m), 7.32-7.36 (2H, m), 7.58-7.63 (2H, m).

### Intermediate 51: (R)-4-Amino-6-(3-fluoro-4-(4,4,5,5-tetramethyl-1,3,2-dioxaborolan-2-yl)phenyl)-8-methyl-7,8-dihydropyrimido[5,4-f][1,4]oxazepin-5(6H)-one

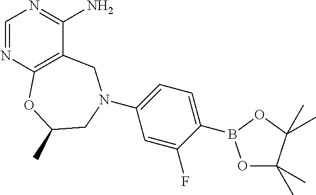

(R)-4-(4-Amino-8-methyl-5-oxo-7,8-dihydropyrimido[5,4-f][1,4]oxazepin-6(5H)-yl)-2-fluorophenyl trifluoromethanesulfonate (Intermediate 51a; 887 mg, 2.03 mmol), (1,1′-bis(diphenylphosphino)ferrocene)-dichloropalladium(II) (DCM adduct) (100 mg, 0.12 mmol), bis(pinacolato)diboron (774 mg, 3.05 mmol) and potassium acetate (618 mg, 6.30 mmol) were suspended in dioxane (20 mL) and sealed into a microwave tube. The tube was degassed under vacuum and the atmosphere replaced with nitrogen. The reaction was heated to 130° C. for 3 hours in the microwave reactor and cooled to RT. The reaction mixture was evaporated to dryness and redissolved in methyl THF (75 mL), filtered through celite and washed sequentially with water (75 mL) and saturated brine (75 mL). The organic layer was dried over Na₂SO₄, filtered and evaporated to afford crude product (924 mg). LCMS showed this to be the desired product mixed with the corresponding boronic acid. Taken on to the next stage without further purification.

### Intermediate 51a: (R)-4-(4-amino-8-methyl-5-oxo-7,8-dihydropyrimido[5,4-f][1,4]oxazepin-6(5H)-yl)-2-fluorophenyl trifluoromethanesulfonate

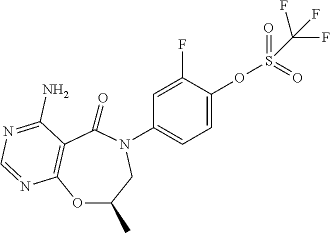

(R)-4-Amino-6-(3-fluoro-4-hydroxyphenyl)-8-methyl-7,8-dihydropyrimido[5,4-f][1,4]oxazepin-5(6H)-one (Intermediate 51b; 0.355 g, 1.17 mmol), 1,1,1-trifluoro-N-phenyl-N-(trifluoromethylsulfonyl)methanesulfonamide (0.834 g, 2.33 mmol) and potassium carbonate (0.484 g, 3.50 mmol) were suspended in THF (210 mL) and sealed into a microwave tube. The reaction was heated to 110° C. for 60 minutes in the microwave reactor and cooled to RT. The suspension was filtered, the solid was washed with ethyacetate (20 mL) and the filtrate was evaporated to dryness and redissolved in DCM (25 mL), and washed with water (25 mL). The organic layer was separated and evaporated to afford crude product. The crude product was purified by ion exchange chromatography, using an SCX column. The desired product was eluted from the column using 0.35M NH₃/MeOH and pure fractions were evaporated to dryness to afford the title compound (0.318 g, 62.5%) as a tan solid. ¹H NMR (400 MHz, DMSO, 30° C.) δ 1.28 (3H, d), 3.83-3.92 (1H, m), 3.93-4.03 (1H, m), 4.85-5.04 (1H, m), 7.38-7.50 (2H, m), 7.64 (1H, s), 7.71-7.86 (2H, m), 8.19 (1H, S). m/z (ES+) (M+H)+=437

### Intermediate 51b: (R)-4-Amino-6-(3-fluoro-4-hydroxyphenyl)-8-methyl-7,8-dihydropyrimido[5,4-f][1,4]oxazepin-5(6H)-one

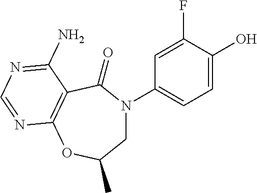

(R)-4-Amino-6-(4-(benzyloxy)-3-fluorophenyl)-8-methyl-7,8-dihydropyrimido[5,4-f][1,4]oxazepin-5(6H)-one (Intermediate 51c; 615 mg, 1.56 mmol) was dissolved in MeOH (20 mL) followed by addition of dihydroxypalladium (219 mg, 0.31 mmol). The reaction mixture was put under H₂ atmosphere (1 atm) and stirred at room temperature for 2 hour. The catalyst was filtered off and rinsed with MeOH. The solid still contained product so was refluxed in methanol (25 mL) for 30 minutes. The catalyst was filtered off and the filtrates were combined and was purified by ion exchange chromatography, using an SCX column. The desired product was eluted from the column using 0.35M NH₃/MeOH and pure fractions were evaporated to dryness to afford (R)-4-amino-6-(3-fluoro-4-hydroxyphenyl)-8-methyl-7,8-dihydropyrimido[5,4-f][1,4]oxazepin-5(6H)-one (363 mg, 77%) as a cream solid. ¹H NMR (400 MHz, DMSO, 30° C.) δ 1.27 (3H, d), 3.68-3.86 (2H, m), 4.79-4.98 (1H, m), 6.87-7.09 (2H, m), 7.15-7.29 (1H, m), 7.26-7.68 (2H, m), 8.18 (1H, s), 9.92 (1H, s). m/z (ES−) (M+H)+=305

### Intermediate 51c: (R)-4-Amino-6-(4-(benzyloxy)-3-fluorophenyl)-8-methyl-7,8-dihydropyrimido[5,4-f][1,4]oxazepin-5(6H)-one

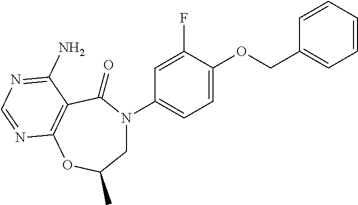

0.5M Ammonia in 1,4-dioxane (100 mL) was added in one portion to (R)-6-(4-(benzyloxy)-3-fluorophenyl)-4-chloro-8-methyl-7,8-dihydropyrimido[5,4-f][1,4]oxazepin-5(6H)-one (Intermediate 51d; 4.45 g, 9.14 mmol) at 20° C. The resulting solution was stirred at 20° C. for 16 hours. The reaction was incomplete and further 0.5M ammonia in 1,4-dioxane (100 mL) added in one portion and the temperature was increased to 45° C. and the reaction mixture was stirred for a further 2 hours. The reaction mixture was allowed to cool and was evaporated. The crude foam was triturated with EtOAc and ether to give (R)-4-amino-6-(4-(benzyloxy)-3-fluorophenyl)-8-methyl-7,8-dihydropyrimido[5,4-f][1,4]oxazepin-5(6H)-one as a solid which was collected by filtration (3.18 g, 8.06 mmol, 88%). The filtrate was evaporated to give a second crop of product (0.622 g, 1.577 mmol, 17.25%) as a pale yellow solid. ¹H NMR (400 MHz, DMSO, 30° C.) δ 1.27 (3H, d), 3.76-3.86 (2H, m), 4.83-4.99 (1H, m), 5.21 (2H, s), 7.13 (1H, ddd), 7.18-7.53 (9H, m), 8.19 (1H, s). m/z (ES+) (M+H)+=395

### Intermediate 51d: (R)-6-(4-(Benzyloxy)-3-fluorophenyl)-4-chloro-8-methyl-7,8-dihydropyrimido[5,4-f][1,4]oxazepin-5(6H)-one

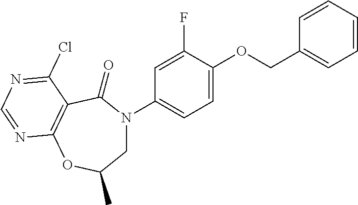

Potassium carbonate (215 mg, 1.55 mmol) was added in one portion to (R) —N-(4-(benzyloxy)-3-fluorophenyl)-4,6-dichloro-N-(2-hydroxypropyl)pyrimidine-5-carboxamide (Intermediate 51e; 350 mg, 0.78 mmol) in DMA (10 mL) at room temperature under air. The resulting suspension was stirred at 100° C. for 2 hours. The reaction mixture was diluted with water (150 mL), and extracted with EtOAc (2×20 mL). The organic phase was washed with water (20 mL), and saturated NaHCO₃ (20 mL). The organic layer was dried over MgSO₄, filtered and evaporated to afford crude product. The crude product was purified by flash silica chromatography, elution gradient 0 to 90% EtOAc in isohexane. Pure fractions were evaporated to dryness to afford the title compound (185 mg, 57.5%) as a cream solid. ¹H NMR (400 MHz, DMSO, 30° C.) δ 1.28 (3H, d), 3.94 (1H, dd), 3.99-4.11 (1H, m), 5.03-5.14 (1H, m), 5.23 (2H, s), 7.21 (1H, ddd), 7.26-7.44 (5H, m), 7.44-7.51 (2H, m), 8.84 (1H, s). m/z (ES+) (M+H)+=414

### Intermediate 51e: (R)—N-(4-(Benzyloxy)-3-fluorophenyl)-4,6-dichloro-N-(2-hydroxypropyl)pyrimidine-5-carboxamide

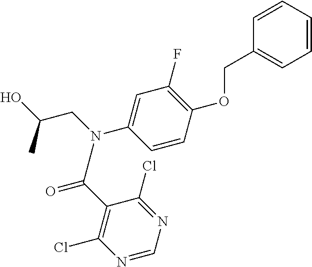

A solution of (R)—N-(4-(benzyloxy)-3-fluorophenyl)-N-(2-(tert-butyldimethylsilyloxy)propyl)-4,6-dichloropyrimidine-5-carboxamide (Intermediate 51f; 24 g, 42.51 mmol) in 4.0 M HCl in dioxane (50 mL) was stirred at 20° C. for 45 minutes. The reaction mixture was evaporated to dryness and redissolved in DCM (150 mL), and washed sequentially with saturated NaHCO₃ (100 mL) and saturated brine (100 mL). The organic layer was separated and filtered through a phase separating funnel and evaporated to afford an orange gum. The crude gum was triturated with EtOAc and isohexane to give a solid which was collected by filtration and dried under vacuum to give an orange solid (not product). The filtrate was evaporated to give crude product as an orange gum. A sample of the crude product was purified by flash silica chromatography, elution gradient 0 to 100% EtOAc in isohexane. Pure fractions were evaporated to dryness to afford the title compound (410 mg, 74.5%) as an orange gum. ¹H NMR (400 MHz, DMSO, 30° C.) δ 1.18 (3H, d), 3.68-3.78 (1H, m), 3.80-3.90 (2H, m), 4.62 (1H, s), 5.16 (2H, s), 7.16-7.29 (3H, m), 7.34-7.54 (5H, m), 8.89 (1H, s). m/z (ES+) (M+H)+=450

### Intermediate 51f: (R)—N-(4-(Benzyloxy)-3-fluorophenyl)-N-(2-(tert-butyldimethylsilyloxy)propyl)-4,6-dichloropyrimidine-5-carboxamide

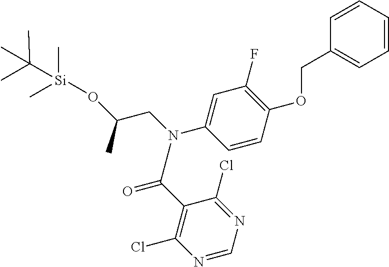

4,6-Dichloropyrimidine-5-carbonyl chloride (12.87 g, 51.75 mmol) as a solution in THF (50 mL) was added dropwise to (R)-4-(benzyloxy)-N-(2-(tert-butyldimethylsilyloxy)propyl)-3-fluoroaniline (19.2 g, 49.28 mmol) and triethylamine (6.14 mL, 44.15 mmol) in THF (150 mL) at 0° C. over a period of 5 minutes under nitrogen. The resulting solution was stirred at 20° C. for 16 hours. The reaction mixture was evaporated to dryness and redissolved in EtOAc (250 mL), and washed sequentially with water (2×150 mL) and saturated brine (75 mL). The organic layer was dried over Na₂SO₄, filtered and evaporated to afford crude title compound (28.4 g, 102%) as an orange oil.

¹H NMR (400 MHz, DMSO, 30° C.) δ −0.04 (6H, d), 0.77 (9H, s), 1.23-1.31 (3H, m), 3.86 (1H, dd), 3.99 (1H, dd), 4.02-4.14 (1H, m), 5.14 (2H, s), 7.08-7.28 (3H, m), 7.33-7.50 (5H, m), 8.89 (1H, d).

### Intermediate 51 g: (R)-4-(Benzyloxy)-N-(2-(tert-butyldimethylsilyloxy)propyl)-3-fluoroaniline

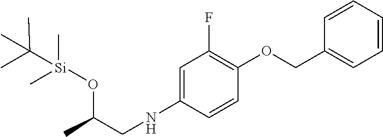

Pd(OAc)₂ (0.799 g, 3.56 mmol) and dicyclohexyl(2′,4′,6′-triisopropylbiphenyl-2-yl)phosphine (1.696 g, 3.56 mmol) were added in one portion to a degassed solution of 1-(benzyloxy)-4-bromo-2-fluorobenzene (10.00 g, 35.57 mmol), (R)-2-(tert-butyldimethylsilyloxy)propan-1-amine (Intermediate 48 g; 8.08 g, 42.69 mmol) and cesium carbonate (17.39 g, 53.36 mmol) in toluene (150 mL) at 20° C. under nitrogen. The resulting suspension was stirred at 120° C. for 35 hours. The reaction mixture was diluted with EtOAc (150 mL) and water (150 mL) and the biphasic mixture was filtered through celite. The organic layer was separated and washed sequentially with water (150 mL) and saturated brine (150 mL). The organic layer was evaporated to afford crude product. The crude product was purified by flash silica chromatography, elution gradient 0 to 20% EtOAc in isohexane. Pure fractions were evaporated to dryness to afford the title compound (9.38 g, 67.7%) as a orange oil. ¹H NMR (400 MHz, DMSO, 30° C.) δ 0.01 (6H, d), 0.86 (9H, s), 1.12 (3H, t), 2.84-2.84 (2H, m), 3.84-4.03 (1H, m), 4.97 (2H, s), 5.37 (1H, t), 6.27 (1H, dd), 6.42 (1H, dd), 6.91 (1H, t), 7.28-7.42 (5H, m). m/z (ES+) (M+H)+=390

### Intermediate 52: (R)-4-(4-Amino-8-methyl-5-oxo-7,8-dihydropyrimido[5,4-f][1,4]oxazepin-6(5H)-yl)-2-cyanophenyl trifluoromethanesulfonate

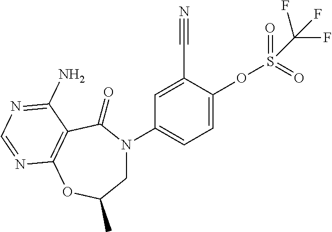

Potassium carbonate (66.6 mg, 0.48 mmol) was added to (R)-5-(4-amino-8-methyl-5-oxo-7,8-dihydropyrimido[5,4-f][1,4]oxazepin-6(5H)-yl)-2-hydroxybenzonitrile (Intermediate 52a; 50 mg, 0.16 mmol) and 1,1,1-trifluoro-N-phenyl-N-(trifluoromethylsulfonyl)methanesulfonamide (57.4 mg, 0.16 mmol) in THF (5 mL) at 20° C. The resulting suspension was stirred at 20° C. for 3 days. The reaction mixture was evaporated to dryness and redissolved in EtOAc (25 mL), and washed sequentially with water (25 mL) and saturated brine (25 mL). The organic layer was dried over Na₂SO₄, filtered and evaporated to afford crude product. The crude product was purified by flash silica chromatography, elution gradient 1 to 8% MeOH in DCM. Pure fractions were evaporated to dryness to afford the title compound (58.0 mg, 81%) as a colourless oil.

¹H NMR (400 MHz, CDCl₃) δ 1.50 (3H, d), 3.90 (2H, m), 4.93 (1H, m), 5.78 (1H, s), 7.56 (1H, d), 7.67 (1H, dd), 7.74 (1H, d), 7.78-7.94 (1H, s), 8.33 (1H, s). m/z (ES+) (M+H)+=444

### Intermediate 52a: (R)-5-(4-Amino-8-methyl-5-oxo-7,8-dihydropyrimido[5,4-f][1,4]oxazepin-6(5H)-yl)-2-hydroxybenzonitrile

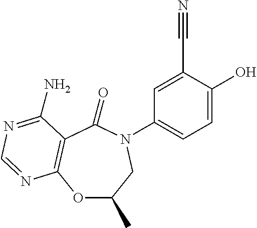

Sodium ethanethiolate (1.544 g, 18.35 mmol) was added to (R)-5-(4-amino-8-methyl-5-s oxo-7,8-dihydropyrimido[5,4-f][1,4]oxazepin-6(5H)-yl)-2-methoxybenzonitrile (Intermediate 52b; 0.597 g, 1.84 mmol) in DMF (20 mL). The resulting solution was stirred at 140° C. for 2 hours. The reaction was cooled and poured into ice/water and neutralised. Extracted into ethyl acetate (2×50 mL) and the combined organics washed with brine (50 mL), dried over Na₂SO₄, filtered and evaporated to give crude product. The crude product was purified by flash silica chromatography, elution gradient 1 to 10% MeOH in DCM. Pure fractions were evaporated to dryness to afford the title compound (0.050 g, 8.75%) as a beige solid. ¹H NMR (400 MHz, DMSO, 30° C.) δ 1.08 (3H, d), 3.61 (2H, m), 4.73 (1H, m), 6.85 (1H, d), 7.32 (3H, m), 7.48 (1H, d), 8.00 (1H, s), 11.03 (1H, s). m/z (ES+) (M+H)+=312

### Intermediate 52b: (R)-5-(4-Amino-8-methyl-5-oxo-7,8-dihydropyrimido[5,4-f][1,4]oxazepin-6(5H)-yl)-2-methoxybenzonitrile

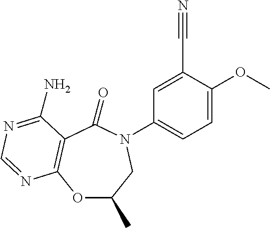

0.5M Ammonia in 1,4-dioxane (50 mL) was added in one portion to (R)-5-(4-chloro-8-methyl-5-oxo-7,8-dihydropyrimido[5,4-f][1,4]oxazepin-6(5H)-yl)-2-methoxybenzonitrile (Intermediate 52c; 0.808 g, 2.34 mmol) at 20° C. The resulting solution was stirred at 55° C. for 24 hours. The reaction mixture was filtered to give a non-product solid which was discarded. The filtrate was evaporated and the resultant solid was triturated with ether to give a solid which was collected by filtration and dried under vacuum to give the title compound (0.760 g, 100%) as a white solid. ¹H NMR (400 MHz, CDCl₃) δ 1.48 (3H, d), 3.75-3.93 (2H, m), 3.97 (3H, s), 4.92 (1H, m), 5.65 (1H, s), 7.04 (1H, dd), 7.49 (1H, d), 7.51 (1H, d), 7.94 (1H, s), 8.32 (1H, s). m/z (ES+) (M+H)+=326.49

### Intermediate 52c: (R)-5-(4-Chloro-8-methyl-5-oxo-7,8-dihydropyrimido[5,4-f][1,4]oxazepin-6(5H)-yl)-2-methoxybenzonitrile

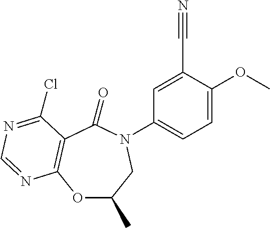

(R)-4,6-Dichloro-N-(3-cyano-4-methoxyphenyl)-N-(2-hydroxypropyl)pyrimidine-5-carboxamide (Intermediate 52d; 2.6 g, 6.82 mmol) and potassium carbonate (2.357 g, 17.05 mmol) were suspended in acetonitrile (20 mL) and sealed into a microwave tube. The reaction was heated to 100° C. for 16 hours in the microwave reactor and cooled to RT. The reaction mixture was evaporated to dryness and redissolved in DCM (75 mL) and washed with water (75 mL). The organic layer was dried over Na₂SO₄, filtered and evaporated to afford crude product. The crude product was purified by flash silica chromatography, elution gradient 20 to 80% EtOAc in isohexane. Pure fractions were evaporated to dryness to afford the title compound (0.808 g, 34.4%) as a white solid.

¹H NMR (400 MHz, CDCl₃) δ 1.45 (3H, d), 3.84 (2H, qd), 3.98 (3H, s), 4.98-5.10 (1H, m), 7.06 (1H, d), 7.56 (1H, d), 7.62 (1H, dd), 8.79 (1H, s). m/z (ES+) (M+H)+=345.38

### Intermediate 52d: (R)-4,6-dichloro-N-(3-cyano-4-methoxyphenyl)-N-(2-hydroxypropyl)pyrimidine-5-carboxamide

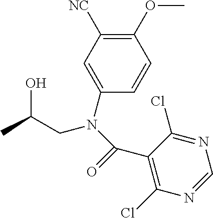

A solution of (R)—N-(2-(tert-butyldimethylsilyloxy)propyl)-4,6-dichloro-N-(3-cyano-4-methoxyphenyl)pyrimidine-5-carboxamide (Intermediate 52e; 4.79 g, 9.67 mmol) in 4.0 M HCl in dioxane (30 mL) was stirred at 20° C. for 45 minutes. The reaction mixture was evaporated to dryness and redissolved in DCM (150 mL), and washed sequentially with saturated NaHCO₃ (100 mL) and saturated brine (100 mL). The organic layer was separated and filtered through a phase separating funnel and evaporated to afford an orange gum. The crude product was purified by flash silica chromatography, elution gradient 40 to 100% EtOAc in isohexane. Pure fractions were evaporated to dryness to afford the title compound (2.60 g, 70.5%) as a white foam. ¹H NMR (400 MHz, CDCl₃, 30° C.) δ 1.29 (3H, t), 1.95 (1H, d), 3.78 (1H, dd), 3.89 (3H, s), 3.91 4.01 (1H, m), 4.15 (1H, dt), 6.84 (1H, d), 7.62 (1H, dt), 7.69 (1H, d), 8.64 (1H, s). m/z (ES+) (M+H)+=381.35

### Intermediate 52e: (R)—N-(2-(tert-Butyldimethylsilyloxy)propyl)-4,6-dichloro-N-(3-cyano-4-methoxyphenyl)pyrimidine-5-carboxamide

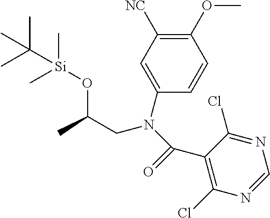

4,6-Dichloropyrimidine-5-carbonyl chloride (4.16 g, 16.71 mmol) as a solution in THF (12.50 mL) was added dropwise to (R)-5-(2-(tert-butyldimethylsilyloxy)propylamino)-2-methoxybenzonitrile (Intermediate 52f; 5.1 g, 15.91 mmol) and triethylamine (2.66 mL, 19.10 mmol) in THF (50 mL) at 0° C. over a period of 5 minutes under nitrogen. The resulting solution was stirred at 20° C. for 16 hours. The reaction mixture was evaporated to dryness and redissolved in EtOAc (250 mL), and washed sequentially with water (2×150 mL) and saturated brine (75 mL). The organic layer was dried over Na₂SO₄, filtered and evaporated to afford crude product. The crude product was purified by flash silica chromatography, elution gradient 10 to 40% EtOAc in isohexane. Pure fractions were evaporated to dryness to afford the title compound (4.79 g, 60.8%) as a pale yellow oil. m/z (ES+) (M+H)+=495.23

### Intermediate 52f: (R)-5-(2-(tert-Butyldimethylsilyloxy)propylamino)-2-methoxybenzonitrile

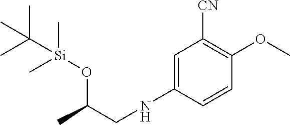

Pd(OAc)₂ (0.529 g, 2.36 mmol) and dicyclohexyl(2′,4′,6′-triisopropylbiphenyl-2-yl)phosphine (1.124 g, 2.36 mmol) were added in one portion to a degassed solution of 5-bromo-2-methoxybenzonitrile (5 g, 23.58 mmol), (R)-2-(tert-butyldimethylsilyloxy)propan-1-amine (Intermediate 48 g; 5.36 g, 28.30 mmol) and cesium carbonate (11.52 g, 35.37 mmol) in toluene (100 mL) at 20° C. under nitrogen. The resulting suspension was stirred at 120° C. for 50 hours. The reaction mixture was diluted with EtOAc (150 mL) and water (150 mL) and the biphasic mixture was filtered through celite. The organic layer was separated and washed sequentially with water (150 mL) and saturated brine (150 mL). The organic layer was evaporated to afford crude product. The crude product was purified by flash silica chromatography, elution gradient 0 to 20% EtOAc in isohexane. Pure fractions were evaporated to dryness to afford the title compound (3.60 g, 47.6%) as a pale yellow oil. ¹H NMR (400 MHz, CDCl₃) 0.05 (3H, s), 0.07 (3H, s), 0.90 (9H, s), 1.21 (3H, d), 2.87-2.97 (1H, m), 3.09 (1H, d), 3.85 (3H, s), 3.99-4.07 (1H, m), 6.69-6.91 (3H, m), 7.63-7.69 (1H, m). m/z (ES+) (M+H)+=321.38

### Intermediate 53: 4-(4-Amino-5-oxo-7,8-dihydropyrimido[5,4-f][1,4]oxazepin-6(5H)-yl)-2-fluorophenyl trifluoromethanesulfonate

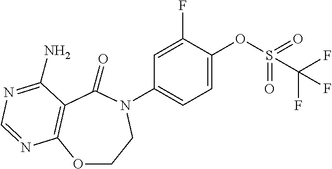

4-Amino-6-(3-fluoro-4-hydroxyphenyl)-7,8-dihydropyrimido[5,4-f][1,4]oxazepin-5(6H)-one (Intermediate 53a; 300 mg, 1.03 mmol), 1,1,1-trifluoro-N-phenyl-N-(trifluoromethylsulfonyl)methanesulfonamide (739 mg, 2.07 mmol) and potassium carbonate (429 mg, 3.10 mmol) were suspended in THF (10 mL) and sealed into a microwave tube. The reaction was heated to 120° C. for 60 minutes in the microwave reactor and cooled to RT. The suspension was poured directly onto a SCX column and the crude reaction mixture was purified by ion exchange chromatography, using an SCX column. The column was first washed with methanol (50 mL) and the desired product was eluted from the column using 0.35M NH₃/MeOH. Pure fractions were evaporated to dryness to afford 4-(4-amino-5-oxo-7,8-dihydropyrimido[5,4-f][1,4]oxazepin-6(5H)-yl)-2-fluorophenyl trifluoromethanesulfonate (323 mg, 74.0%) as a tan solid. ¹H NMR (400 MHz, DMSO, 30° C.) δ 3.94 4.11 (2H, m), 4.52-4.69 (2H, m), 7.36-7.50 (1H, m), 7.64 (2H, s), 7.70-7.85 (2H, m), 8.16 (1H, s). m/z (ES+) (M+H)+=423

### Intermediate 53a: 4-Amino-6-(3-fluoro-4-hydroxyphenyl)-7,8-dihydropyrimido[5,4-f][1,4]oxazepin-5(6H)-one

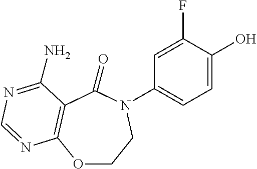

4-Amino-6-(4-(benzyloxy)-3-fluorophenyl)-7,8-dihydropyrimido[5,4-f][1,4]oxazepin-5(6H)-one (Intermediate 53b; 1 g, 2.63 mmol) was dissolved in MeOH (100 mL) followed by addition of dihydroxypalladium (0.369 g, 0.53 mmol). The reaction mixture was put under H₂ atmosphere (1 atm) and stirred at room temperature for 2 hour. The catalyst was filtered off and rinsed with MeOH. The solid still contained product so was heated in methanol (25 mL) for 30 minutes. The catalyst was filtered off and the filtrates were combined and the solvent was removed under reduced pressure to give 4-amino-6-(3-fluoro-4-hydroxyphenyl)-7,8-dihydropyrimido[5,4-f][1,4]oxazepin-5(6H)-one (0.520 g, 68.1%) as a cream solid. ¹H NMR (400 MHz, DMSO, 30° C.) δ 3.84-4.01 (2H, m), 4.51-4.65 (2H, m), 6.89-7.07 (2H, m), 7.18-7.32 (1H, m), 7.61 (2H, s), 8.17 (1H, s), 9.96 (1H, s). m/z (ES+) (M+H)+=291

### Intermediate 53b: 4-Amino-6-(4-(benzyloxy)-3-fluorophenyl)-7,8-dihydropyrimido[5,4-f][1,4]oxazepin-5(6H)-one

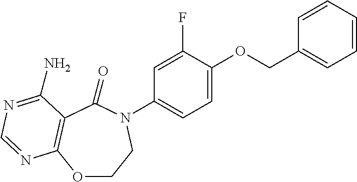

0.5M ammonia in 1,4-dioxane (100 mL) was added in one portion to 6-(4-(benzyloxy)-3-fluorophenyl)-4-chloro-7,8-dihydropyrimido[5,4-f][1,4]oxazepin-5(6H)-one (Intermediate 53c; 1.04 g, 2.60 mmol) at 20° C. The resulting solution was stirred at 45° C. for 16 hours. The reaction mixture was filtered to give a solid (not product). The filtrate was evaporated. The crude solid was triturated with EtOAc and ether to give a solid which was collected by filtration and dried under vacuum to give 4-amino-6-(4-(benzyloxy)-3-fluorophenyl)-7,8-dihydropyrimido[5,4-f][1,4]oxazepin-5(6H)-one (1.05 g, 106%) as a cream solid.

¹H NMR (400 MHz, DMSO, 30° C.) δ 3.82-4.02 (2H, m), 4.50-4.67 (2H, m), 5.21 (2H, s), 7.13 (1H, ddd), 7.27 (1H, t), 7.32-7.49 (6H, m), 7.60 (2H, s), 8.15 (1H, s). m/z (ES+) (M+H)+=381

### Intermediate 53c: 6-(4-(benzyloxy)-3-fluorophenyl)-4-chloro-7,8-dihydropyrimido[5,4-f][1,4]oxazepin-5(6H)-one

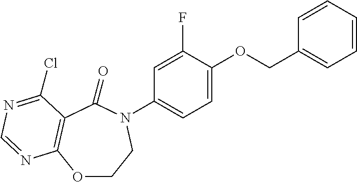

Triethylamine (3.65 ml, 26.26 mmol) was added in one portion to N-(4-(benzyloxy)-3-fluorophenyl)-4,6-dichloro-N-(2-hydroxyethyl)pyrimidine-5-carboxamide (Intermediate 53 d; 3.35 g, 6.91 mmol) in acetonitrile (100 ml) at 20° C. under nitrogen. The resulting solution was stirred at 80° C. for 16 hours. The reaction mixture was evaporated to dryness and redissolved in DCM (100 mL), and washed sequentially with water (100 mL) and saturated brine (100 mL). The organic layer was dried over MgSO₄, filtered and evaporated to afford crude product. The crude residue was triturated with EtOAc and ether. No solid precipitated. Left to stand overnight to give a solid which was collected by filtration and dried under vacuum to give 6-(4-(benzyloxy)-3-fluorophenyl)-4-chloro-7,8-dihydropyrimido[5,4-f][1,4]oxazepin-5(6H)-one (1.05 g, 2.63 mmol, 38.0%) as a tan solid. On standing a second crop was obtained (0.66 g, 1.651 mmol, 23.89%)

¹H NMR (400 MHz, DMSO, 30° C.) δ 4.06 4.23 (2H, m), 4.63 4.81 (2H, m), 5.23 (2H, s), 7.18 7.23 (1H, m), 7.27 7.50 (7H, m), 8.81 (1H, s). m/z (ES+) (M+H)+=400

### Intermediate 53d: N-(4-(benzyloxy)-3-fluorophenyl)-4,6-dichloro-N-(2-hydroxyethyl)pyrimidine-5-carboxamide

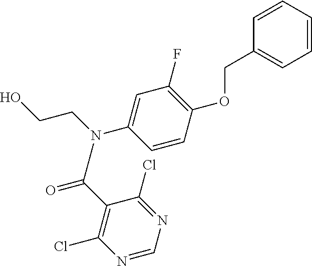

Conc hydrochloric acid (5 mL) in methanol (20 mL) was added in one portion to a solution of N-(4-(benzyloxy)-3-fluorophenyl)-N-(2-(tert-butyldimethylsilyloxy)ethyl)-4,6-dichloropyrimidine-5-carboxamide (Intermediate 53e; 4.90 g, 8.90 mmol) in methanol (100 mL) at 20° C. under nitrogen. The resulting solution was stirred at 20° C. for 60 minutes. The reaction mixture was evaporated to dryness and redissolved in EtOAc (150 mL), and washed sequentially with saturated NaHCO₃ (100 mL) and saturated brine (100 mL). The organic layer was dried over Na₂SO₄, filtered and evaporated to afford an orange gum. The crude gum was triturated with EtOAc and isohexane. No solid was formed. The reaction mixture was evaporated to afford N-(4-(benzyloxy)-3-fluorophenyl)-4,6-dichloro-N-(2-hydroxyethyl)pyrimidine-5-carboxamide (3.31 g, 85%) as a orange gum. This was used without further purification. ¹H NMR (400 MHz, DMSO, 30° C.) δ 3.58 3.66 (2H, m), 3.85 3.94 (2H, m), 4.89 (1H, s), 5.14 (2H, d), 7.20 7.29 (3H, m), 7.34 7.53 (5H, m), 8.88 (1H, s). m/z (ES+) (M+H)+=436

### Intermediate 53e: N-(4-(benzyloxy)-3-fluorophenyl)-N-(2-(tert-butyldimethylsilyloxy)ethyl)-4,6-dichloropyrimidine-5-carboxamide

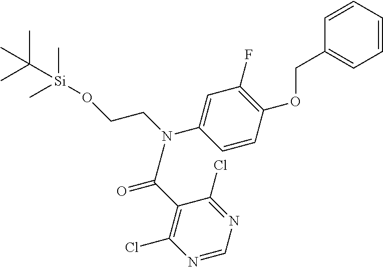

4,6-Dichloropyrimidine-5-carbonyl chloride (1.7 g, 7.24 mmol) was added dropwise to 4-s (benzyloxy)-N-(2-(tert-butyldimethylsilyloxy)ethyl)-3-fluoroaniline (Intermediate 53f; 3.02 g, 7.24 mmol) and Et₃N (1.448 mL, 10.39 mmol) in THF (100 mL) at 0° C. under argon. Et₃N (1.448 mL, 10.39 mmol) was added dropwise to the reaction mixture at 0° C. under nitrogen. The resulting suspension was stirred at 0° C. for 16 hours. The reaction mixture was evaporated to dryness and redissolved in EtOAc (150 mL), and washed sequentially with water (2×150 mL) and saturated brine (75 mL). The organic layer was dried over Na₂SO₄, filtered and evaporated to afford crude product N-(4-(benzyloxy)-3-fluorophenyl)-N-(2-(tert-butyldimethylsilyloxy)ethyl)-4,6-dichloropyrimidine-5-carboxamide (4.91 g, 123%) as an orange oil. ¹H NMR (400 MHz, DMSO, 30° C.) δ −0.00 (6H, s), 0.82 (9H, d), 3.71-3.78 (2H, m), 3.91 (2H, t), 5.09 (2H, s), 7.06-7.23 (2H, m), 7.29-7.41 (6H, m), 8.83 (1H, s). m/z (ES+) (M+H)+=550

### Intermediate 53f: 4-(Benzyloxy)-N-(2-(tert-butyldimethylsilyloxy)ethyl)-3-fluoroaniline

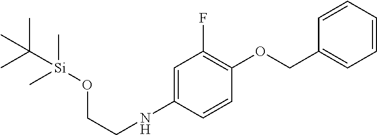

Pd(OAc)₂ (0.399 g, 1.78 mmol) and dicyclohexyl(2′,4′,6′-triisopropylbiphenyl-2-yl)phosphine (0.848 g, 1.78 mmol) were added in one portion to a degassed solution of 1-(benzyloxy)-4-bromo-2-fluorobenzene (5.00 g, 17.79 mmol), 2-(tert-butyldimethylsilyloxy)ethanamine (4.16 g, 17.79 mmol) and cesium carbonate (8.69 g, 26.68 mmol) in toluene (100 mL) at 20° C. in a microwave vial. The microwave vial was sealed and the reaction was heated to 120° C. for 10 hours in the microwave reactor and cooled to RT. The mixture was then split equally into 5×20 ml microwave vials and each was heated to 120° C. for 10 hours in the microwave reactor then cooled to RT. The reaction mixtures were combined and diluted with EtOAc (150 mL), and washed sequentially with water (200 mL) and saturated brine (200 mL). The organic layer was filtered and dried over Na₂SO₄, filtered and evaporated to afford crude product. The crude product was evaporated and was purified by flash silica chromatography, elution gradient 0 to 30% EtOAc in isohexane. Pure fractions were evaporated to dryness to afford 4-(benzyloxy)-N-(2-(tert-butyldimethylsilyloxy)ethyl)-3-fluoroaniline (3.62 g, 54.2%) as a yellow oil.

¹H NMR (400 MHz, DMSO, 30° C.) δ −0.00 (6H, s), 0.80-0.86 (9H, m), 3.06 (2H, q), 3.61-3.70 (2H, m), 4.96 (2H, s), 5.36 (1H, t), 6.23-6.32 (1H, m), 6.44 (1H, dd), 6.90 (1H, tt), 7.25 7.44 (5H, m).

1. A compound of formula (I) or a pharmaceutically-acceptable salt thereof: 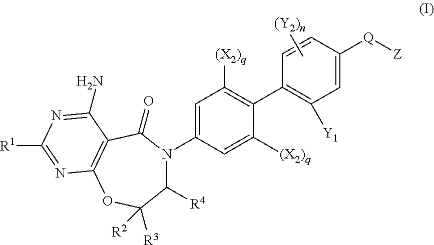
wherein
R¹ is selected from hydrogen, (1-4C)alkyl, (1-4C)alkoxy, (1-4C)perfluoroalkyl, and (1-4C)perfluoroalkoxy;
R² and R³ are independently selected from hydrogen, (1-4C)alkyl and (1-4C)perfluoroalkyl, or R² and R³ together with the carbon to which they are attached from a (3-6C)cycloalkyl ring;
R⁴ is selected from hydrogen and (1-4C)alkyl;
each q is independently 0 or 1 and each X₂ is independently selected from fluoro, chloro, bromo, amino, cyano, (1-3C)alkyl, (2-3C)alkenyl, (2-3C)alkynyl and (1-2C)alkoxy;
Y₁ is selected from hydrogen, fluoro, chloro, bromo, cyano, (1-3C)alkyl and (1-2C)alkoxy;
n is 0, 1 or 2 and each Y₂ is independently selected from fluoro, chloro, bromo, cyano, hydroxy, (1-3C)alkyl and (1-2C)alkoxy;
Q is selected from a direct bond, —(CR⁵R⁶)ₚ—, —O—(CR⁵R⁶)_{q}—, —C(O)—(CR⁵R⁶)ₜ— and —(CR⁵R⁶)ᵣ₁—O—(CR⁵R⁶)ᵣ₂— wherein p is 1, 2 or 3, q and t are independently 0, 1 or 2, r1 and r2 are independently 0 or 1, and R⁵ and R⁶ are independently selected from hydrogen, methyl and ethyl;
Z is selected from hydrogen, hydroxyl, fluoro, chloro, bromo and cyano or is selected from one of the following eight groups:
(a) —CONR⁷R⁸ wherein R⁷ and R⁸ are independently selected from hydrogen, (1-3C)alkyl, —(CR⁵R⁶)ᵤ(3-5C)cycloalkyl, —(CR⁵R⁶)ₛNR⁹R¹⁰, —(CR⁵R⁶)ₛ₁—O—(CR⁵R⁶)ₛ₂NR⁹R¹⁰, —(CR⁵R⁶)ᵥ-(4- to 7-membered heterocyclyl ring) and —(CR⁵R⁶)_{w}-(5- to 7-membered heteroaryl ring) or R⁷ and R⁸ together with the nitrogen to which they are attached form a 4- to 7-membered heterocyclic ring, 7- to 8-membered spirocyclic heterocyclic ring system, or 6- to 10-membered fused bicyclic heterocyclic ring system, wherein any ring or ring system is optionally substituted with one or two groups independently selected from oxo, hydroxyl, hydroxy(1-3C)alkyl, methoxy, amino, N-(1-3C)alkylamino and N,N-di(1-3C)alkylamino;
wherein:
the alkyl, cycloalkyl and heterocyclyl are optionally substituted by hydroxyl, (1-4C)alkanoyl or methoxy, and the cycloalkyl and heterocyclyl are optionally substituted by (1-4C)alkyl; and the heteroaryl ring is optionally substituted by fluoro, chloro, cyano, methyl, trifluoromethyl or difluoromethyl;
s is independently 1, 2 or 3
s1 and s2 are independently 2 or 3;
u, v and w are independently 0, 1, 2 or 3;
R⁵ and R⁶ are as defined above;
R⁹ and R¹⁰ are independently selected from hydrogen, (1-3C)alkyl, (1-6C)alkoxycarbonyl, (3-5C)cycloalkyl and a 3- to 5-membered heterocyclyl ring, or R⁹ and R¹⁰ together with the nitrogen to which they are attached form a 4- to 7-membered heterocyclic ring optionally substituted with one or two groups independently selected from (1-4C)alkyl, hydroxy(1-4C)alkyl, oxo, (1-4C)alkanoyl, hydroxy and methoxy;
(b) —SO₂NR^{7a}R^{8a}, wherein R^{7a} and R^{8a} are independently selected from hydrogen and variables defined above for R⁷ and R⁸;
(c) —S(O)ₜR⁷, wherein R⁷ is as defined above (excluding hydrogen) and t is 0, 1 or 2;
(d) —NR⁷COR⁸ wherein R⁷ and R⁸ are as defined above or R⁷ and R⁸ together form a 2-oxo-substituted 5- to 7-membered heterocyclyl ring optionally substituted with one or two substituents independently selected from hydroxyl, (1-4C)alkyl, (1-4C)alkanoyl and methoxy;
(e) —NR⁷SO₂R⁸ wherein R⁷ and R⁸ are as defined above or R⁷ and R⁸ together form a S,S-dioxo-substituted 5- to 7-membered heterocyclyl ring optionally substituted with one or two substituents independently selected from hydroxyl, (1-4C)alkyl, (1-4C)alkanoyl and methoxy;
(f) —NR⁷R⁸ wherein R⁷ and R⁸ are as defined above or R⁷ and R⁸ together with the nitrogen to which they are attached form a 5- to 7-membered heterocyclyl ring optionally substituted with one or two substituents independently selected from hydroxyl, (1-4C)alkyl, (1-4C)alkanoyl and methoxy;
(g) —OR⁷ wherein R⁷ is as defined above (excluding hydrogen);
(h) —S(O)═NR¹¹ wherein R¹¹ is H or methyl;
and wherein any carbon atom in a linear (1-3C)alkyl, (1-3C)alkyl or (1-2C)alkoxy containing group defined above may be optionally substituted by up to 3 fluoro atoms;
with the provisos that:
(i) within the definition of Q, when q is 0 or r2 is 0 then Z cannot be hydroxyl or —OR⁷; and
(ii) when Z is bromo or chloro then Q must be a direct bond.

2. The compound of formula (I) as claimed in claim 1, or a pharmaceutically-acceptable salt thereof, wherein
R¹ is selected from hydrogen, (1-4C)alkyl, (1-4C)alkoxy, (1-4C)perfluoroalkyl, and (1-4C)perfluoroalkoxy;
R² and R³ are independently selected from hydrogen, (1-4C)alkyl and (1-4C)perfluoroalkyl, or R² and R³ together with the carbon to which they are attached from a (3-6C)cycloalkyl ring;
R⁴ is selected from hydrogen and (1-4C)alkyl;
each q is independently 0 or 1 and each X₂ is independently selected from fluoro, chloro, bromo, amino, cyano, (1-3C)alkyl, (2-3C)alkenyl, (2-3C)alkynyl and (1-2C)alkoxy;
Y₁ is selected from hydrogen, fluoro, chloro, bromo, cyano, (1-3C)alkyl and (1-2C)alkoxy;
n is 0, 1 or 2 and each Y₂ is independently selected from fluoro, chloro, bromo, cyano, hydroxy, (1-3C)alkyl and (1-2C)alkoxy;
Q is selected from a direct bond, —(CR⁵R⁶)ₚ—, —O—(CR⁵R⁶)_{q}—, —C(O)—(CR⁵R⁶)ₜ— and —(CR⁵R⁶)ᵣ₁—O—(CR⁵R⁶)ᵣ₂— wherein p is 1, 2 or 3, q and t are independently 0, 1 or 2, r1 and r2 are independently 0 or 1, and R⁵ and R⁶ are independently selected from hydrogen, methyl and ethyl;
Z is selected from hydrogen, hydroxyl, fluoro, chloro, bromo and cyano, or is selected from one of the following eight groups:
(a) —CONR⁷R⁸ wherein R⁷ and R⁸ are independently selected from hydrogen, (1-3C)alkyl, —(CR⁵R⁶)ᵤ(3-5C)cycloalkyl, —(CR⁵R⁶)ₛNR⁹R¹⁰, —(CR⁵R⁶)ₛ₁—O—(CR⁵R⁶)ₛ₂NR⁹R¹⁰, —(CR⁵R⁶)ᵥ-(4- to 7-membered heterocyclyl ring) and —(CR⁵R⁶)_{w}-(5- to 7-membered heteroaryl ring) or R⁷ and R⁸ together with the nitrogen to which they are attached form a 4- to 7-membered heterocyclic ring, 7- to 8-membered spirocyclic heterocyclic ring system, or 6- to 10-membered fused bicyclic heterocyclic ring system, wherein any ring or ring system is optionally substituted with one or two groups independently selected from oxo, hydroxyl, hydroxy(1-3C)alkyl, methoxy, amino, N-(1-3C)alkylamino and N,N-di(1-3C)alkylamino;
wherein:
the alkyl, cycloalkyl and heterocyclyl are optionally substituted by hydroxyl, (1-4C)alkanoyl or methoxy, and the cycloalkyl and heterocyclyl are optionally substituted by (1-4C)alkyl; and the heteroaryl ring is optionally substituted by fluoro, chloro, cyano, methyl, trifluoromethyl or difluoromethyl;
s, s1 and s2 are independently 2 or 3;
u, v and w are independently 0, 1, 2 or 3;
R⁵ and R⁶ are as defined above;
R⁹ and R¹⁰ are independently selected from hydrogen, (1-3C)alkyl, (1-6C)alkoxycarbonyl, (3-5C)cycloalkyl and a 3- to 5-membered heterocyclyl ring, or R⁹ and R¹⁰ together with the nitrogen to which they are attached form a 4- to 7-membered heterocyclic ring optionally substituted with one or two groups independently selected from (1-4C)alkyl, hydroxy(1-4C)alkyl, oxo, (1-4C)alkanoyl, hydroxy and methoxy;
(b) —SO₂NR^{7a}R^{8a}, wherein R^{7a} and R^{8a} are independently selected from hydrogen and variables defined above for R⁷ and R⁸;
(c) —S(O)ₜR⁷, wherein R⁷ is as defined above (excluding hydrogen) and t is 0, 1 or 2;
(d) —NR⁷COR⁸ wherein R⁷ and R⁸ are as defined above or R⁷ and R⁸ together form a 2-oxo-substituted 5- to 7-membered heterocyclyl ring,
(e) —NR⁷SO₂R⁸ wherein R⁷ and R⁸ are as defined above or R⁷ and R⁸ together form a S,S-dioxo-substituted 5- to 7-membered heterocyclyl ring optionally substituted with one or two substituents independently selected from hydroxyl, (1-4C)alkyl, (1-4C)alkanoyl and methoxy;
(f) —NR⁷R⁸ wherein R⁷ and R⁸ are as defined above or R⁷ and R⁸ together with the nitrogen to which they are attached form a 5- to 7-membered heterocyclyl ring optionally substituted with one or two substituents independently selected from hydroxyl, (1-4C)alkyl, (1-4C)alkanoyl and methoxy;
(g) —OR⁷ wherein R⁷ is as defined above (excluding hydrogen);
(h) —S(O)═NR¹¹ wherein R¹¹ is H or methyl;
and wherein any carbon atom in a linear (1-3C)alkyl, (1-3C)alkyl or (1-2C)alkoxy containing group defined above may be optionally substituted by up to 3 fluoro atoms;
with the proviso that:
(i) when q is 0 or r2 is 0 then Z cannot be hydroxyl or —OR⁷;
or a pharmaceutically-acceptable salt thereof.

3. The compound according to claim 1, or a pharmaceutically-acceptable salt thereof, wherein R¹, R³, and R⁴ are all hydrogen and R² is hydrogen or methyl.

4. The compound according to claim 1, or a pharmaceutically-acceptable salt thereof, wherein Y₁ is hydrogen, fluoro, chloro, cyano, methyl or trifluoromethyl.

5. The compound according to claim 1, or a pharmaceutically-acceptable salt thereof, wherein one q is 1 and X₂ is fluoro.

6. The compound according to claim 1, or a pharmaceutically-acceptable salt thereof, wherein Q is a direct bond or —CH₂—.

7. The compound according to claim 1, or a pharmaceutically-acceptable salt thereof, wherein R¹, R³ and R⁴ are all hydrogen;
R² is hydrogen or methyl;
one q=1 and the other q=0;
X₂ is fluoro or cyano;
Y₁ is chloro;
n is 0 or 1 and Y₂ is selected from fluoro, chloro and (1-3C)alkyl;
Q-Z is hydrogen, methyl, fluoro or chloro.

8. The compound according to claim 7, or a pharmaceutically-acceptable salt thereof, which is a compound of formula (IA):

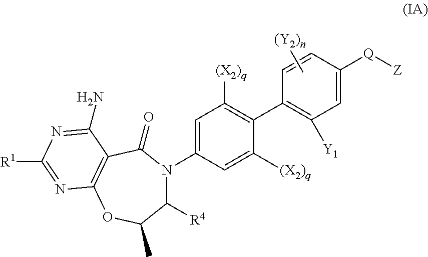

9. The compound according to claim 1, or a pharmaceutically-acceptable salt thereof, wherein R¹, R², R³ and R⁴ are all hydrogen;
each q is 0;
Y₁ is selected from fluoro, chloro and (1-3C)alkyl;
n is 0 or 1 and Y₂ is selected from fluoro, chloro and (1-3C)alkyl;
Z is selected from hydrogen, fluoro, chloro and cyano;
Q is a direct bond or —CH₂—.

10. A pharmaceutical composition which comprises a compound of formula (I) as claimed in claim 1, or a pharmaceutically-acceptable salt thereof, in association with a pharmaceutically-acceptable excipient or carrier.

11.-14. (canceled)

15. A method of treating diabetes mellitus and/or obesity in a warm-blooded animal in need of such treatment which comprises administering to said animal an effective amount of a compound of formula (I) as claimed in claim 1, or a pharmaceutically-acceptable salt thereof.

16. A method for producing an inhibition of DGAT1 activity in a warm-blooded animal in need of such treatment which comprises administering to said animal an effective amount of a compound of formula (I) as claimed in claim 1, or a pharmaceutically-acceptable salt thereof.

17. The method of claim 15, wherein the warm-blooded animal is a human being.
